(19) United States
(12) Patent Application Publication
(10) Pub. No.: US 2013/0225586 A1
(43) Pub. Date: Aug. 29, 2013

(54) COMPOUNDS, COMPOSITIONS, PROCESSES OF MAKING, AND METHODS OF USE RELATED TO INHIBITING MACROPHAGE MIGRATION INHIBITORY FACTOR

(71) Applicant: CPSI Stockholder Trust, (US)

(72) Inventors: Thais Sielecki-Dzurdz, Kennett Square, PA (US); Vidal F. de la Cruz, Phoenixville, PA (US)

(73) Assignee: CPSI STOCKHOLDER TRUST, New York, NY (US)

(21) Appl. No.: 13/626,816

(22) Filed: Sep. 25, 2012

Related U.S. Application Data
(63) Continuation of application No. 11/742,978, filed on May 1, 2007, now abandoned.
Application No. 11/742,978 is a continuation of application No. 11/090,128, filed on Mar. 28, 2005, now abandoned.
(60) Provisional application No. 60/556,440, filed on Mar. 26, 2004.

(51) Int. Cl. C07D 413/06; C07D 413/04; C07D 261/04

(52) U.S. Cl. CPC C07D 413/06; C07D 261/04; C07D 413/04
USPC 514/236.8; 548/240; 514/378; 544/137; 544/367; 514/254.04; 435/375; 435/366

(57) ABSTRACT

The present invention provides a compound having Formula I or II:

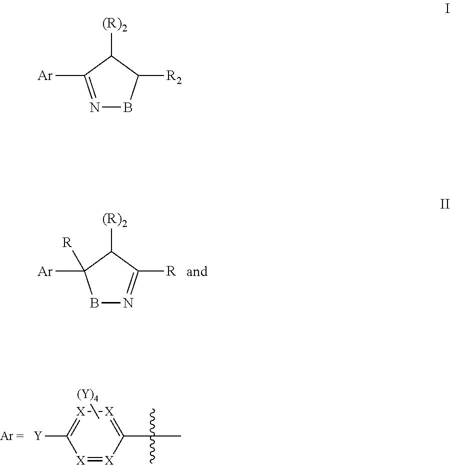

wherein B, R, X, Ar, and Y are defined herein, pharmaceutically acceptable salts thereof and pharmaceutically acceptable prodrugs thereof. The present invention also provides methods of making and using the compound.

### CROSS-REFERENCE TO RELATED APPLICATIONS

This application claims priority to U.S. Provisional Application Ser. No. 60/556,440, filed Mar. 26, 2004, the entire contents of which are hereby incorporated by reference.

### BACKGROUND OF THE INVENTION

1. Field of the Invention

The present invention relates to isoxazoline and related compounds, to intermediates and methods for their preparation, to compositions containing them, and to their use.

2. Background of the Technology

Macrophage migration inhibitory factor (MIF) is one of the earliest described cytokines, and is an immunoregulatory protein with a wide variety of cellular and biological activities (for reviews see: Swope, et al., Rev. Physiol. Biochem. Pharmacol. 139, 1-32 (1999); Metz, et al., Adv. Immunol. 66, 197-223 (1997); and Bucala, FASEB J. 14, 1607-1613 (1996)). Originally, MIF was found to be secreted by activated lymphoid cells, to inhibit the random migration of macrophages, and to be associated with delayed-type hypersensitivity reactions (George, et al., Proc. Soc. Exp. Biol. Med., 111, 514-521 (1962); Weiser, et al., J. Immunol. 126, 1958-1962 (1981); Bloom, et al., Science, 153:80-82 (1966); David, Proc. Natl. Acad. Sci. USA, 56, 72-77 (1966). MIF was also shown to enhance macrophage adherence, phagocytosis and tumoricidal activity (Nathan et al., J. Exp. Med., 137, 275-288 (1973); Nathan, et al., J. Exp. Med., 133, 1356-1376 (1971); Churchill, et al., J. Immunol., 115, 781-785 (1975)). The availability of recombinant MIF has allowed for confirmation of these biological activities, and for the identification of additional activities.

Recombinant human MIF was originally cloned from a human T cell library (Weiser, et al., Proc. Natl. Acad. Sci. USA, 86, 7522-7526 (1989)), and was shown to activate blood-derived macrophages to kill intracellular parasites and tumor cells in vitro, to stimulate IL-1β and TNFα expression, and to induce nitric oxide synthesis (Weiser. et al., J. Immunol., 147, 2006-2011 (1991); Pozzi, et al., Cellular Immunol., 145, 372-379 (1992); Weiser, et al., Proc. Natl. Acad. Sci. USA, 89, 8049-8052 (1992); Cunha, et al., J. Immunol., 150, 1908-1912 (1993)). While the conclusions available from several of these early reports are confounded by the presence of a bioactive mitogenic contaminant in the recombinant MIF preparations used, the potent pro-inflammatory activities of MIF have been established in other studies that do not suffer from this complicating factor (reviewed in Bucala, The FASEB, Journal 10, 1607-1613 (1996)).

More recent MIF studies have capitalized on the production of recombinant MIF in purified form as well as the development of MIF-specific polyclonal and monoclonal antibodies to establish the biological role of MIF in a variety of normal homeostatic and pathophysiological settings (reviewed in Rice, et al., Annual Reports in Medicinal Chemistry, 33, 243-252 (1998)). Among the most important insights of these later reports has been the recognition that MIF not only is a cytokine product of the immune system, but also is a hormone-like product of the endocrine system, particularly the pituitary gland. This work has underscored the potent activity of MIF as a counter-regulator of the anti-inflammatory effects of the glucocorticoids (both those endogenously released and those therapeutically administered), with the effect that the normal activities of glucocorticoids to limit and suppress the severity of inflammatory responses are inhibited by MIF. The endogenous MIF response is thus seen as a cause or an exacerbative factor in a variety of inflammatory diseases and conditions (reviewed in Donnelly, et al., Molecular Medicine Today, 3, pp. 502-507 (1997)).

MIF is now known to have several biological functions beyond its well-known association with delayed-type hypersensitivity reactions. For example, as mentioned above, MIF released by macrophages and T cells acts as a pituitary mediator in response to physiological concentrations of glucocorticoids (Bucala, FASEB J., 14, 1607-1613 (1996)). This leads to an overriding effect of glucocorticoid immuno-suppressive activity through alterations in TNF-α, IL-1B, IL-6, and IL-8 levels. Additional biological activities of MIF include the regulation of stimulated T cells (Bacher, et al., Proc. Natl. Acad. Sci. USA, 93, 7849-7854 (1996)), the control of IgE synthesis (Mikayama, et al., Proc. Natl. Acad. Sci. USA, 90, 10056-10060 (1993)), the functional inactivation of the p53 tumor suppressor protein (Hudson, et al., J. Exp. Med., 190, 1375-1382 (1999)), the regulation of glucose and carbohydrate metabolism (Sakaue, et al., Mol. Med., 5, 361-371 (1999)), and the attenuation of tumor cell growth and tumor angiogenesis (Chesney, et al., Mol. Med., 5, 181-191 (1999); Shimizu, et al., Biochem. Biophys. Res. Commun., 264, 751-758 (1999)).

Interleukin-1 (IL-1) and Tumor Necrosis Factor (TNF) are biological substances produced by a variety of cells, such as monocytes or macrophages. IL-1 has been demonstrated to mediate a variety of biological activities thought to be important in immunoregulation and other physiological conditions such as inflammation. The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis, osteoarthritis, endotoxemia and/or toxic shock syndrome, other acute or chronic inflammatory disease states such as the inflammatory reaction induced by endotoxin or inflammatory bowel disease, tuberculosis, atherosclerosis, diabetes, muscle degeneration, cachexia, psoriatic arthritis, Reiter's syndrome, rheumatoid arthritis, gout, traumatic arthritis, rubella arthritis, and acute synovitis.

Excessive or unregulated TNF production has been implicated in mediating or exacerbating a number of diseases including rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, fever and myalgias due to infection, such as influenza, cachexia secondary to infection or malignancy, cachexia secondary to acquired immune deficiency syndrome (AIDS), AIDS, ARC (AIDS related complex), keloid information, scar tissue formation, Crohn's disease, ulcerative colitis, or pyrosis.

Interleukin-8 (LL-8) is a chemotactic factor produced by several cell types including mononuclear cells, fibroblasts, endothelial cells, and keratinocytes. Its production from endothelial cells is induced by IL-1, TNF, or lipopolysaccharide (LPS). IL-8 stimulates a number of functions in vitro. It has been shown to have chemoattractant properties for neutrophils, T-lymphocytes, and basophils. In addition it induces histamine release from basophils from both normal and atopic individuals as well lysosomal enzyme release and respiratory burst from neutrophils. IL-8 has also been shown to increase the surface expression of Mac-1 (CD11b/CD18) on neutrophils without de novo protein synthesis, this may contribute to increased adhesion of the neutrophils to vascular endothelial cells. Many diseases are characterized by massive neutrophil infiltration. Conditions associated with an increase in IL-8 production (which is responsible for chemotaxis of neutrophils into the inflammatory site) would benefit by compounds which are suppressive of IL-8 production.

IL-1 and TNF affect a wide variety of cells and tissues and these cytokines as well as other leukocyte derived cytokines are important and critical inflammatory mediators of a wide variety of disease states and conditions. The inhibition of these cytokines is of benefit in controlling, reducing and alleviating many of these disease states.

The three-dimensional crystal structure of human MIF reveals that the protein exists as a homotrimer (Lolis, et al., Proc. Ass. Am. Phys., 108, 415-419 (1996) and is structurally related to 4-oxalocrotonate tautomerase, 5-carboxymethyl-2-hydroxymuconate, chorismate mutase, and to D-dopachrome tautomerase (Swope, et al., EMBO J., 17, 3534-3541 (1998); Sugimoto, et al., Biochemistry, 38, 3268-3279 (1999). Recently, the crystal structure has been reported for the complex formed between human MIF and p-hydroxyphenylpyruvic acid (Lubetsky, et al., Biochemistry, 38, 7346-7354 (1999). It was found that the substrate binds to a hydrophobic cavity at the amino terminus and interacts with Pro-1, Lys-32, and Ile-64 in one of the subunits, and with Tyr-95 and Asn-97 in an adjacent subunit. Similar interactions between murine MIF and (E)-2-fluoro-p-hydroxycinnamate have been reported (Taylor, et al., Biochemistry, 38, 7444-7452 (1999)). Solution studies using NMR provide further evidence of the interaction between p-hydroxyphenylpyruvic acid and Pro-1 in the amino-terminal hydrophobic cavity (Swope, et al., EMBO J., 17, 3534-3541 (1998)).

Mutation studies provide convincing evidence that Pro-1 is involved in the catalytic function of MIF. Deletion of Pro-1 or replacement of Pro-1 with Ser (Bendrat, et al., Biochemistry, 36, 15356-15362 (1997)), Gly (Swope, et al., EMBO J., 17, 3534-3541 (1998)), or Phe (Hermanowski-Vosatka, et al., Biochemistry, 38, 12841-12849 (1999)), and addition of an N-terminal peptide tag to Pro-1 (Bendrat, et al., Biochemistry, 36, 15356-15362 (1997)) abrogated the catalytic activity of MIF in assays using L-dopachrome methyl ester and p-hydroxyphenyl-pyruvic acid. A similar loss in activity was found by inserting Ala between Pro-1 and Met-2 (Lubetsky et al., Biochemistry, 38, 7346-7354 (1999). The Pro to Ser MIF mutant showed glucocorticoid counter-regulatory activity (Bendrat, et al., Biochemistry, 36, 15356-15362 (1997)) and was fully capable, as was the Pro to Phe mutant, of inhibiting monocyte chemotaxis (Hermanowski-Vosatka et al., Biochemistry, 38, 12841-12849 (1999). In contrast, the Pro to Gly MIF mutant was greatly impaired in its ability to stimulate superoxide generation in activated neutrophils (Swope et al., EMBO J., 17, 3534-3541 (1998).

MIF has been characterized as an anterior pituitary-derived hormone that potentiates lethal endotoxemia (Bucala, Immunol. Lett., 1994, 43, 23-26; Bucala, Circ. Shock, 1994, 44, 35-39), a factor which can override glucocorticoid-mediated suppression of inflammatory and immune responses (Calandra and Bucala, Crit. Rev. Immol., 1997, 17, 77-88; Calandra and Bucala, J. Inflamm., 1995, 47, 39-51), and as an activator of T-cells after mitogenic or antigenic stimuli (Bacher et al., Proc. Natl. Acad. Sci. U.S.A., 1996, 93, 7849-7854).

This cytokine has been shown to have multiple roles within the confines of regulating the immune response as well as being associated with cell growth and differentiation during wound repair and carcinogenesis. Expression has been shown to be elevated in prostate adenocarcinomas (Arcuri et al., Prostate, 1999, 39, 159-165; Meyer-Siegler and Hudson, Urology, 1996, 48, 448-452), colon carcinomas of the mouse (Takahashi et al., Mol. Med., 1998, 4, 707-714), lipopolysacharide-induced HL60 cells (a leukemia cell line) (Nishihira et al., Biochem. Mol. Biol. Int., 1996, 40, 861-869), and upon treatment with ultraviolet radiation (Shimizu et al., J. Invest. Dermatol., 1999, 112, 210-215). The pharmacological modulation of MIF activity and/or expression may therefore be an appropriate point of therapeutic intervention in pathological conditions.

The protein has been detected in the synovia of patients with rheumatoid arthritis (Onodera et al., Cytokine, 1999, 11, 163-167) and its expression at sites of inflammation and from macrophages suggests a role for the mediator in regulating the function of macrophages in host defense (Calandra et al., J. Exp. Med., 1994, 179, 1895-1902). Activity of MIF has also been found to correlate well with delayed hypersensitivity and cellular immunity in humans (Bernhagen et al., J. Exp. Med., 1996, 183, 277-282; David, Proc. Natl. Acad. Sci. U.S.A., 1966, 56, 72-77). The protein has also been implicated in neural function and development in rodents (Bacher et al., Mol. Med., 1998, 4, 217-230; Matsunaga et al., J. Biol. Chem., 1999, 274, 3268-3271; Nishio et al., Biochim. Biophys. Acta., 1999, 1453, 74-82; Suzuki et al., Brain Res., 1999, 816, 457-462).

There is a need in the art to discover and develop small organic molecules that function as MIF inhibitors (e.g., antagonists) and further possess the benefits of small organic molecule therapeutics versus larger, polymeric protein (e.g., antibody) and nucleic acid-based (e.g., anti-sense) therapeutic agents. The therapeutic potential of low molecular weight MIF inhibitors is substantial, given the activities of anti-MIF antibodies in models of endotoxin- and exotoxin-induced toxic shock (Bernhagen et al., Nature, 365, 756-759 (1993); Kobayashi et al., Hepatology, 29, 1752-1759 (1999); Calandra et al., Proc. Natl. Acad. Sci. USA., 95, 11383-11388 (1998); and Makita et al., Am. J. Respir. Crit. Care Med. 158, 573-579 (1998), T-cell activation (Bacher et al., Proc. Natl. Acad. Sci. USA., 93, 7849-7854 (1996), autoimmune diseases (e.g., graft versus host disease, insulin-dependent diabetes, and various forms of lupus) including rheumatoid arthritis (Kitaichi, et al., Curr. Eye Res., 20, 109-114 (2000); Leech, et al., Arthritis Rheum., 42, 1601-1608 (1999), wound healing (Abe, et al., Biochim. Biophys. Acta, 1500, 1-9 (2000), and angiogenesis (Shimizum, et al., Biochem. Biophys. Res. Commun., 264, 751-758 (1999). Low molecular weight anti-MIF drugs exhibiting such activities may offer clinical advantages over neutralizing antibodies and nucleic acid-based agents because they may be orally active or generally more easily administered, have better bioavailabilities, have improved biodistribution, and are normally much less expensive to produce.

### RELATED ART

U.S. Pat. No. 4,933,464 to Markofsky discloses a process for forming 3-phenylisoxazolines and 3-phenylisoxazoles and related products.

U.S. Pat. No. 6,114,367 to Cohan et al. discloses isoxazoline compounds which are inhibitors of tumor necrosis factor (TNF). The isoxazoline compounds are said to be useful for inhibiting TNF in a mammal in need thereof and in the treatment or alleviation of inflammatory conditions or disease. Also disclosed are pharmaceutical compositions comprising such compounds.

Curuzu et al., Collect. Czech. Chem. Commun., 56: 2494-2499 (1991) discloses 3-substituted phenyl-4,5-dihydroisoxazoleneacetic acids, including 3-(4-hydroxyphenyl)-4,5-dihydro-5-isoxazolineacetic acid and 3-(4-methoxyphenyl)-4,5-dihydro-5-isoxazolineacetic acid, and shows that the first of these two compounds is devoid of anti-inflammatory activity, while the second is dramatically reduced in such activity compared to the parent compound that was unsubstituted in the para position of the phenyl ring, in a carageenin-induced edema assay in the rat paw.

Wityak et al., J. Med. Chem., 40: 50-60 (1997) discloses isoxazoline antagonists of the glycoprotein IIb/IIIa receptor.

Kleinman, et al., “Striking effect of hydroxamic acid substitution on the phosphodiesterase type 4 (PDE4) and TNF alpha inhibitory activity of two series of rolipram analogues: implications for a new active site model of PDE4”. J. Med. Chem. 41(3): 266-270 (1998), discloses inter alia the following compounds: [3-(3-cyclopentyloxy-4-methoxy-phenyl)-4,5-dihydro-isoxazol-5-yl]-acetic acid and the methyl ester thereof, as well as [3-(3-cyclopentyloxy-4-methoxy-phenyl)-4,5-dihydro-isoxazol-5-yl]-N-hydroxy-acetamide.

U.S. Pat. No. 6,492,428, to Al-Abed et al. issued Dec. 10, 2003, and discloses quinone-related compounds having MIF inhibitor activity.

U.S. Pat. No. 6,599,938, to Al-Abed et al. issued Jul. 29, 2003, and discloses amino acid/benzaldehyde Schiff base compounds having MIF inhibitor activity.

U.S. Pat. No. 6,599,903, to de Lassauniere et al. issued Jul. 29, 2003, and discloses compounds in pharmaceutical compositions.

U.S. Pat. No. 6,630,461 to de Lassauniere et al. issued Oct. 7, 2003, and discloses compounds in pharmaceutical compositions.

United States Patent Appln. Pub. No. 2003/0008908 to Al-Abed published on Jan. 9, 2003 and discloses compounds in pharmaceutical compositions.

Any disclosure cited herein is incorporated by reference in its entirety for all purposes.

### SUMMARY OF THE INVENTION

One embodiment of the present invention provides a compound having Formula I or II:

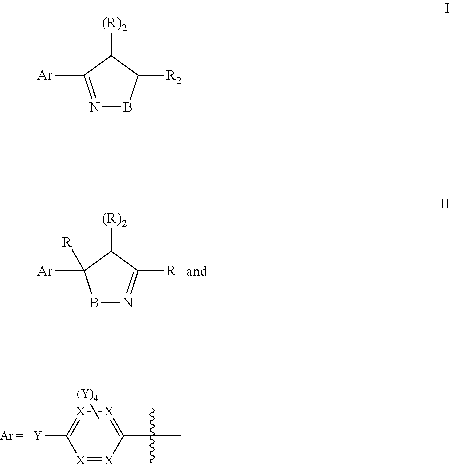

wherein B is oxygen or sulphur; and

each R is independently defined as follows:

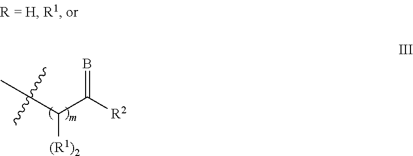

wherein in Formula I and Formula II, at least one R is not hydrogen;

wherein each R¹ is independently hydrogen, an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteraryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group;

each R² is independently an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteroaryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group

each m is independently zero or an integer from one to twenty; and

each X is independently carbon or nitrogen, wherein when any X is carbon, then each Y is defined independently as follows:

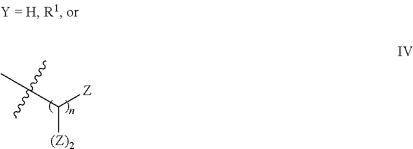

wherein each Z is independently hydrogen, an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteroaryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group; and

each n is independently zero or an integer from one to four;

pharmaceutically acceptable salts thereof and pharmaceutically acceptable prodrugs thereof.

One embodiment of the present invention provides a method, which includes inhibiting the production of at least one cytokine selected from the group including MIF, IL-1, IL-2, IL-6, IL-8, IFN-γ, TNF, and a combination thereof in a mammalian subject in need thereof by administering an inhibiting-effective amount of the above compound to the subject.

Another embodiment of the present invention provides a method, which includes inhibiting an ERIC/MAP pathway in a mammalian subject in need thereof by administering an inhibiting-effective amount of the above compound to the subject.

### DESCRIPTION OF THE FIGURES

Various other objects, features, and attendant advantages of the present invention will be more fully appreciated as the same becomes better understood from the following detailed description when considered in connection with the accompanying drawings in which like reference characters designate like or corresponding parts throughout the several views and wherein:

FIG. 1A shows one synthetic scheme for synthesizing Phenyl Series A Compounds according to one embodiment of the invention;

FIG. 1B shows one synthetic scheme for synthesizing Phenyl Series B Compounds according to one embodiment of the invention;

FIG. 2A shows one synthetic scheme for synthesizing Propyl Series A Compounds according to one embodiment of the invention;

FIG. 2B shows one synthetic scheme for synthesizing Propyl Series B Compounds according to one embodiment of the invention;

FIG. 3A shows one synthetic scheme for synthesizing Butyl Series A Compounds according to one embodiment of the invention;

FIG. 3B shows one synthetic scheme for synthesizing Butyl Series B Compounds according to one embodiment of the invention; and

FIG. 4 shows one synthetic scheme for synthesizing Furyl Series Compounds according to one embodiment of the invention.

### DETAILED DESCRIPTION OF THE EMBODIMENTS

A more complete appreciation of the invention and many of the attendant advantages thereof will be readily obtained as the same becomes better understood by reference to the following detailed description when considered with the accompanying drawings.

The present invention relates to isoxazoline and related compounds, to intermediates and methods for their preparation, to compositions containing them and to their use. More particularly, the present invention relates to pharmaceutical compositions containing the subject compounds, and medicinal uses of the subject compounds and compositions. Even more particularly, the present invention may be suitably used for the prevention and treatment of various conditions in humans.

One aspect of the present invention provides for a genus of isoxazoline and isoxazoline-related compounds, pharmaceutical compositions and related methods of making and their use in treatments and diagnostics. The compounds have macrophage migration inhibitory factor (MIF) antagonist activity, and related activities with other cytokines affected by MIF activity. The compounds act as inhibitors of MIF, and also modulating other cytokines affected by MIF activity including IL-1, IL-2, IL-6, IL-8, IFN-γ and TNF. The compounds and compositions are useful for treating a variety of diseases involving any disease state in a human, or other mammal, which is exacerbated by or caused by excessive or unregulated MIF, IL-1, IL-2, IL-6, IL-8, IFN-γ and TNF production by such mammal's cells, such as, but not limited to, monocytes and/or macrophages, or any disease state that is modulated by inhibiting the ERK/MAP pathway.

In the following chemical formulae, the use of the superscript on a substituent is to identify a substituent name (e.g., “R²” is used to indicate an R²-named substituent), while the use of a subscript is used to enumerate the number of times a substituent occurs at that molecular site (e.g., “R₂” or “(R)₂” both are used to indicate two substituents simply named as “R”).

The present invention relates to a compound of general Formula I or II

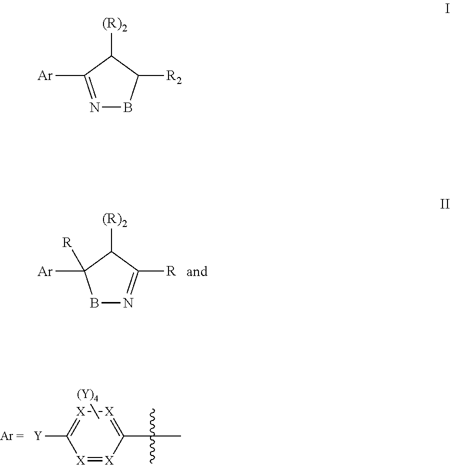

wherein B is either oxygen or sulphur and each “R” is independently defined:

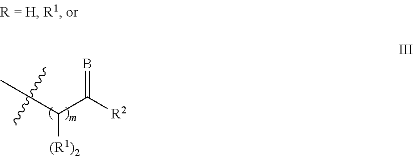

with the requirement that each “R” cannot only occur as hydrogen on either Formula I or II (i.e., at least one R on either Formula I or II is an “R” substituent other than hydrogen), and any B is independently either oxygen or sulphur; any R′ is independently hydrogen, (C₁-C₆)alkyl or some other suitable substituent, any R² is an amine, an alkoxy or some other suitable substituent; and “m” is independently either zero or an integer from one to twenty;

each X is independently either carbon or nitrogen; and when any X is carbon, then Y is the substituent defined independently for each X as

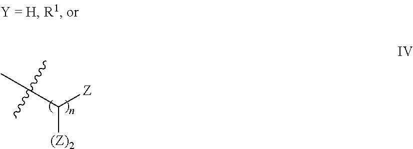

each Z is independently either hydrogen, hydroxyl, halogen, or some other suitable substituent; and

“n” is independently either zero or an integer from one to four;

and pharmaceutically acceptable salts and prodrugs thereof.

In one embodiment, for compounds having Formulas I and II hereinabove and below, when the ring “X” is nitrogen instead of carbon, then that X nitrogen does not bear a Y. For example, in this embodiment, the number of Y groups may correspond to the number of X carbons, i.e., a number of 1, 2, 3 or 4.

In one embodiment, the present invention excludes compounds within general Formula I and having a chemical structure falling within Formula IA:

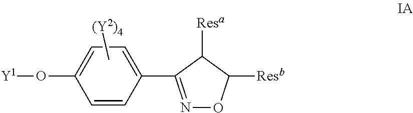

wherein

each Y¹ is independently a hydrogen or (C₁-C₆)alkyl;

each Y² is independently a Y¹, hydroxyl, halo, —N₃, —CN, —SH, or —N(Y¹)₂;

Res^{a} is independently a Y¹, halo, —N₃, —CN, —OY¹, —N(Y¹)₂, —SH, ═O, ═CH₂, or A, and each A is independently either phenyl or an aromatic ring substituted with one or more independent Y² substituents; Res^{b} is defined as follows:

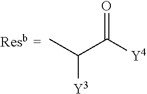

Y³ is independently a Y¹, A, —(CH₂)-A, —N(Y¹)₂, or —NY¹Y⁵, with each Y⁵ being a saturated or unsaturated, straight or branched (C₂-C₁₈)alkyl; and

Y⁴ is independently a Y¹, —OY¹, —OY⁵, —N(Y¹)₂, —NY¹Y⁵, or A.

The present invention also relates to the pharmaceutically acceptable acid addition salts of the compounds of general Formula I or IL

The compounds of the Formula I or II which are basic in nature are capable of forming a wide variety of different salts with various inorganic and organic acids. Although such salts must be pharmaceutically acceptable for administration to animals, it is often desirable in practice to initially isolate a compound of the Formula I or II from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained. The acids which are used to prepare the pharmaceutically acceptable acid addition salts of the aforementioned base compounds of this invention include those which form non-toxic acid addition salts, i.e., salts containing pharmacologically acceptable anions, such as the chloride, bromide, iodide, nitrate, sulfate, bisulfate, phosphate, acid phosphate, acetate, lactate, citrate, acid citrate, tartrate, bitartrate, succinate, maleate, fumarate, glutamate, L-lactate, L-tartrate, tosylate, mesylate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compound. The chemical bases that may be used as reagents to prepare pharmaceutically acceptable base salts of those compounds of general Formula I or II that are acidic in nature are those that form non-toxic base salts with such compounds. Those compounds of the Formula I or II which are also acidic in nature, e.g., where substituent R, R¹, R², or R³ includes a —COOH or tetrazole moiety, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention include those which form non-toxic base salts with the herein described acidic compounds of Formula I or II. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The compounds and prodrugs of the present invention can exist in several tautomeric forms, and geometric isomers and mixtures thereof. All such tautomeric forms are included within the scope of the present invention. Tautomers exist as mixtures of tautomers in solution. In solid form, usually one tautomer predominates. Even though one tautomer may be described, the present invention includes all tautomers of the present compounds.

The present invention also includes atropisomers of the present invention. Atropisomers refer to compounds of the invention that can be separated into rotationally restricted isomers. The compounds of this invention may contain olefin-like double bonds. When such bonds are present, the compounds of the invention exist as cis and trans configurations and as mixtures thereof.

The present invention also includes isotopically-labeled compounds, which are identical to those recited in general Formulas I or II, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula I or II of this invention and prodrugs thereof can generally be prepared by carrying out the procedures disclosed herein, e.g., in the Examples, by substituting a readily available isotopically labeled reagent for a non-isotopically labeled reagent.

A “suitable substituent” is intended to mean a chemically and pharmaceutically acceptable functional group i.e., a moiety that does not negate the inhibitory activity of the inventive compounds. Such suitable substituents may be routinely selected by those skilled in the art. Illustrative examples of suitable substituents include, but are not limited to halo groups, perfluoroalkyl groups, perfluoroalkoxy groups, alkyl groups, cycloalkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, arylsulfonyl groups and the like.

More specifically, the present invention also relates to a compound having the general Formula I or II

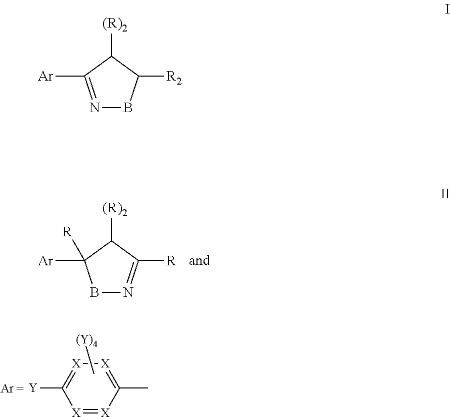

wherein B is either oxygen or sulphur and each “R” is independently defined:

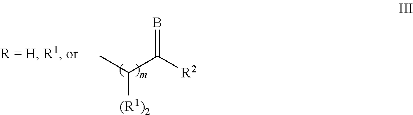

with the requirement that each “R” can never occur only as hydrogen on either Formula I or II, and further, that within each “R” independently, any B is either oxygen or sulphur; and “m” is independently either zero or an integer from one to twenty; each X is independently either carbon or nitrogen; and when any X is carbon, then Y is the substituent defined independently for each X as

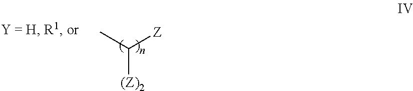

each Z is independently either hydrogen, hydroxyl, fluorine, bromine, iodine, —N₃, —CN, —SR³, —OR³, —N(R¹)₂, —R¹, or A, and

“n” is independently either zero or an integer from one to four;

each R¹ is independently selected from hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; (C₁-C₁₀)heteroaryl-O—, (C₁-C₁₀)heterocyclic-O—, (C₃-C₁₀)cyclo alkyl-O—, (C₁-C₆)alkyl-S—, (C₁-C₆)alkyl-SO₂—, (C₁-C₆)alkyl-NH—SO₂—, —NO₂, amino, (C₁-C₆)alkyl-amino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl—(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂ —N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—-O— and phenyl-(C═O)—O—, wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, NO₂, amino, (C₁-C₆)alkyl-amino, [(C₁-C₆)alkyl]₂-amino, (C₁-C₆)alkyl-SO₂—NH—, (C₁-C₆)alkyl-(C═O)—NH—, (C₁-C₆)alkyl-(C═O)-[((C₁-C₆)alkyl)-N]—, phenyl-(C═O)—NH—, phenyl-(C═O)-[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂—N—(C═O)—, phenyl-NH—(C═O)—, phenyl-[((C₁-C₆)alkyl)-N]—(C═O)—, (C₁-C₁₀)heteroaryl-NH—-(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two independently chosen R¹ alkyl-containing groups may be taken together with any nitrogen atom to which they are attached to form a three to forty membered cyclic, heterocyclic or heteroaryl ring;

each R² is independently selected from the group consisting of hydrogen, hydroxyl, halo, —N₃, —CN, —SH, (R¹)₂—N—, (R³)—O—, (R³)—S—, (C₁-C₆)alkyl, (C₂-C₆)alkenyl, (C₂-C₆)alkYnYl, (C₃-C₁₀)cycloalkyl, phenyl, (C₁-C₁₀)heteroaryl, and (C₁-C₁₀)hetero-cyclic; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂—N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)-[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)-[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)-[((C₁-C₆)alkyl)-N]—, (phenyl-)₂N—(C═O)-[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—-(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂ N—(C═O)—O—; wherein when said R² phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;

each R³ is independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, NO₂, amino, (C₁-C₆)allyl-amino, [(C₁-C₆)alkyl]₂-amino, (C₁-C₆)alkyl-SO₂—NH—, (C₁-C₆)alkyl-(C═O)—NH—, (C₁-C₆)allyl-(C═O)-[((C₁-C₆)alkyl)-N]—, phenyl-(C═O)—NH—, phenyl-(C═O)—[(C₁-C₆)alkyl)-N], —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂—N—(C═O)—, phenyl-NH—(C═O)—, phenyl-[((C₁-C₆)alkyl)-N]—-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—;

or the pharmaceutically acceptable salts and prodrugs thereof.

As used herein, the term “alkyl,” as well as the alkyl moieties of other groups referred to herein (e.g., alkoxy), may be linear or branched (such as methyl, ethyl, n-propyl, isopropyl, n-butyl, iso-butyl, secondary-butyl, tertiary-butyl), and they may also be cyclic (e.g., cyclopropyl or cyclobutyl); optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said alkyl” as used herein refers to any of the preceding alkyl moieties within a group such alkoxy, alkenyl or alkylamino. Preferred alkyls include (C₁-C₄)alkyl, most preferably methyl.

As used herein, the term “cycloalkyl” refers to a mono or bicyclic carbocyclic ring (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1-2 double bonds and optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (Cᵣ C₆)alkyl. The phrase “each of said alkyl” as used herein refers to any of the preceding alkyl moieties within a group such alkoxy, alkenyl or alkylamino. Preferred cycloalkyls include cyclobutyl, cyclopentyl and cyclohexyl.

As used herein, the term “halogen” or “halo” includes fluoro, chloro, bromo or iodo or fluoride, chloride, bromide or iodide.

As used herein, the term “halo-substituted alkyl” refers to an alkyl radical as described above substituted with one or more halogens included, but not limited to, chloromethyl, dichloromethyl, fluoromethyl, difluoromethyl, trifluoromethyl, 2,2,2-trichloroethyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “alkenyl” means straight or branched chain unsaturated radicals of 2 to 6 carbon atoms, including, but not limited to ethenyl, 1-propenyl, 2-propenyl(allyl), iso-propenyl, 2-methyl-1-propenyl, 1-butenyl, 2-butenyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “(C₂-C₆)alkynyl” is used herein to mean straight or branched hydrocarbon chain radicals having one triple bond including, but not limited to, ethynyl, propynyl, butyryl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “carbonyl” or “(C═O)” (as used in phrases such as alkylcarbonyl, alkyl-(C═O)— or alkoxycarbonyl) refers to the joinder of the >C═O moiety to a second moiety such as an alkyl or amino group (i.e., an amido group). Alkoxycarbonylamino (i.e., alkoxy(C═O)—NH—) refers to an alkyl carbamate group. The carbonyl group is also equivalently defined herein as (C═O). Alkylcarbonylamino refers to groups such as acetamide.

As used herein, the term “phenyl-[(C₁-C₆)alkyl)-N]—(C═O)—,” refers to a disubstituted amide group of the formula:

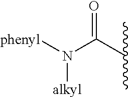

As used herein, the term “aryl” means aromatic radicals such as phenyl, naphthyl, tetrahydronaphthyl, indanyl and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “heteroaryl” refers to an aromatic heterocyclic group with at least one heteroatom selected from O, S and N in the ring. In addition to said heteroatom, the aromatic group may optionally have up to four N atoms in the ring. For example, heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like; optionally substituted by i to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

The term “heterocyclic” as used herein refers to a cyclic group containing 1-9 carbon atoms and 1-4 hetero atoms selected from N, O, S or NR′. Examples of such rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl and the like. Examples of such monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

Another embodiment of the present invention includes those compounds having a chemical structure within one of the following two formulas:

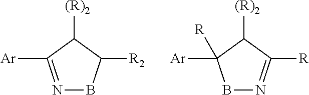

wherein R and B are defined as in general Formula I and II above with the exception that at least one R in each above chemical structure formula contains one of the two following chemical sub-structures

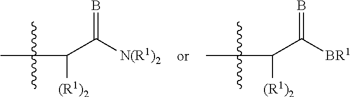

and Ar is either one of the following eight chemical sub-structures

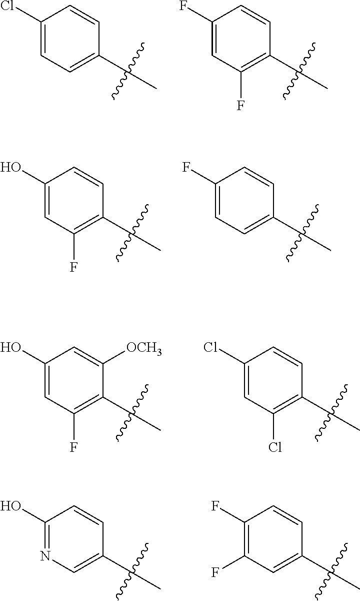

or Ar is defined as one of the following three chemical sub-structures

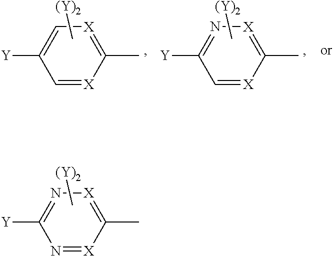

wherein each X is independently either carbon or nitrogen; and when any X is carbon, then Y is the substituent defined independently for each X as

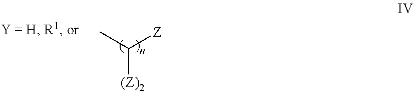

each Z is independently either hydrogen, hydroxyl, fluorine, bromine, iodine, —N₃, —CN, —SR³, —OR³, —N(R¹)₂, “n” is independently either zero or an integer from one to four; and R¹ and R³ are defined as in general Formula I or II. A preferred embodiment here is wherein B is oxygen and/or from the R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered, cyclic heterocyclic or heteroaryl ring. Still more preferred are when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

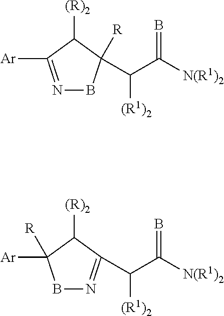

wherein Ar, R, B and R¹ are as defined in general Formula I and H above. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered, cyclic, heterocyclic or heteroaryl ring. Still more preferred are when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

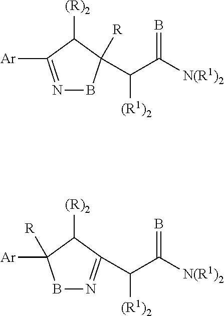

wherein R and B are as defined in general Formula I or II above,
and Ar is either one of the following eight chemical sub-structures

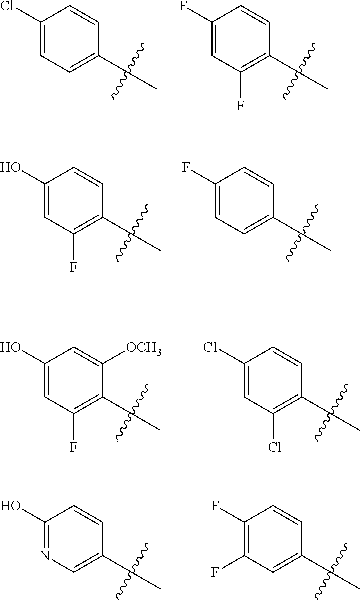

or Ar is defined as one of the following three chemical sub-structures

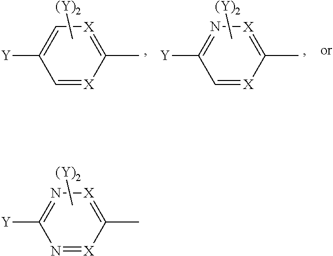

wherein each X is independently either carbon or nitrogen; and when any X is carbon, then Y is the substituent defined independently for each X as

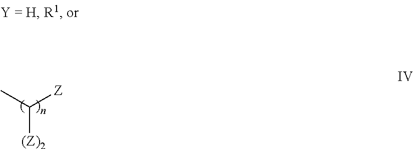

each Z is independently either hydrogen, hydroxyl, fluorine, bromine, iodine, —N₃, —CN, —SR³, —OR³, —N(R¹)₂, “n” is independently either zero or an integer from one to four; and R¹ and R³ are defined as in general Formula I or II. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, C₁-C₆)alkyl-S—; wherein two independently chosen R¹ alkyl-containing groups may be taken together with any nitrogen atom to which they are attached to form a three to forty membered, cyclic, heterocyclic or heteroaryl ring. Still more preferred is when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

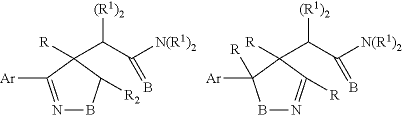

wherein Ar, R, B and R¹ are as defined in general Formula I and II above. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered, cyclic, heterocyclic or heteroaryl ring. Still more preferred are when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

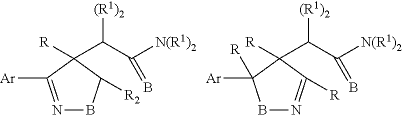

wherein R is defined as in general Formula I and II above and Ar is either one of the following eight chemical sub-structures

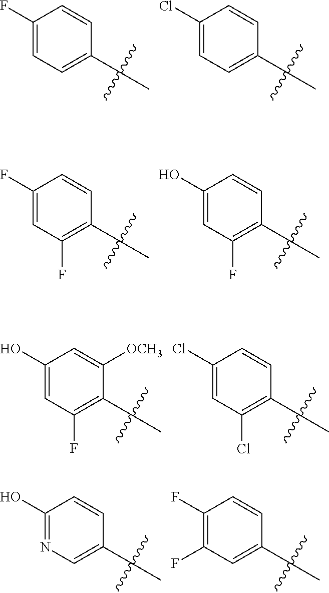

or Ar is defined as one of the following three chemical sub-structures

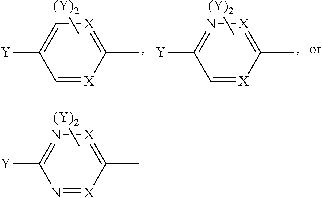

wherein each X is independently either carbon or nitrogen; and when any X is carbon, then Y is the substituent defined independently for each X as

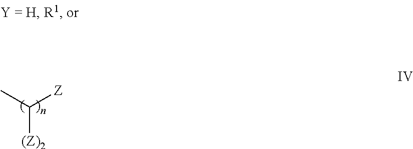

each Z is independently either hydrogen, hydroxyl, fluorine, bromine, iodine, —N₃, —CN, —SR³, —OR³, —N(R¹)₂, “n” is independently either zero or an integer from one to four; and R¹ and R³ are defined as in general Formula I or II. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered, cyclic, heterocyclic or heteroaryl ring. Still more preferred are when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

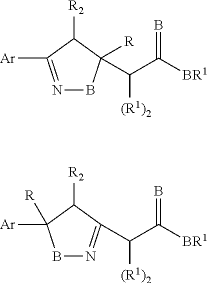

wherein Ar, R, B and R¹ are as defined in general Formula I and II above. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered heterocyclic or heteroaryl ring. Still more preferred are when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

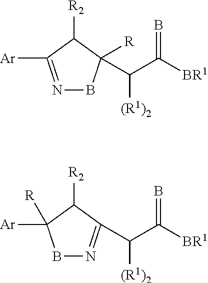

wherein R and B are as defined in general Formula I or II above,
Ar is either one of the following eight chemical sub-structures

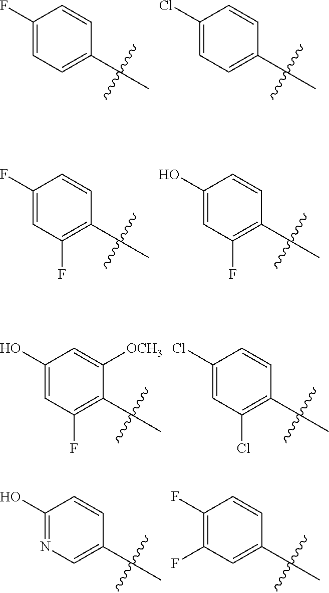

or Ar is defined as one of the following three chemical sub-structures

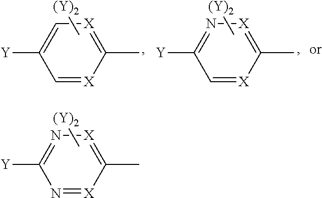

wherein each X is independently either carbon or nitrogen; and when any X is carbon, then Y is the substituent defined independently for each X as

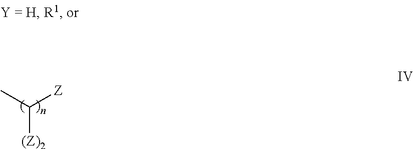

each Z is independently either hydrogen, hydroxyl, fluorine, bromine, iodine, —N₃, —CN, —SR³, —N(R¹)₂, “n” is independently either zero or an integer from one to four; and R¹ and R³ are defined as in general Formula I or II. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered, cyclic, heterocyclic or heteroaryl ring. Still more preferred is when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

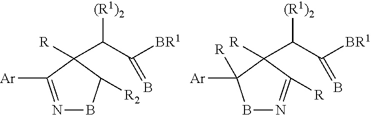

wherein Ar, R, B and R¹ are as defined in general Formula I and II above. A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered cyclic, heterocyclic or heteroaryl ring. Still more preferred is when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Another embodiment of the present invention includes those compounds having a chemical structure within one of the following two formulas:

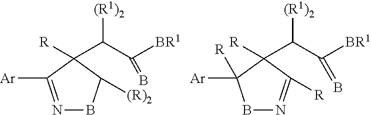

wherein R is defined as in general Formula I and II above and Ar is either one of the following eight chemical sub-structures

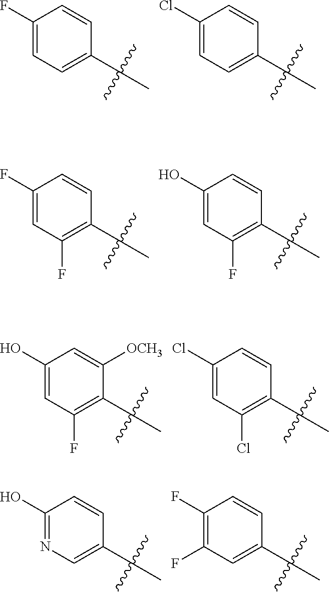

or Ar is defined as one of the following three chemical sub-structures

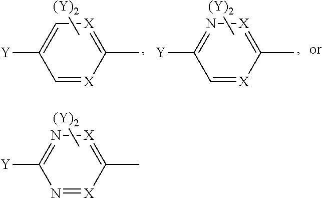

wherein each X is independently either carbon or nitrogen; and when any X is carbon, then Y is the substituent defined independently for each X as

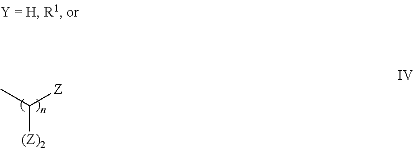

each Z is independently either hydrogen, hydroxyl, fluorine, bromine, iodine, —N₃, —CN, —SR³, —N(R¹)₂, “n” is independently either zero or an integer from one to four; and R¹ and R³ are defined as in general Formula I or II A preferred embodiment here is wherein B is oxygen and/or R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered cyclic heterocyclic or heteroaryl ring. Still more preferred is when R and R¹ are defined as independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy. Even still more preferred is when R and R¹ are defined as independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl. Most preferred is when R and R¹ are defined as independently selected from the group consisting of hydrogen, (C₃-C₆)cycloalkyl, (C₁-C₆)alkoxy, and (C₁-C₆)alkyl.

Other embodiments of the present invention relate to those compounds described above or listed in TABLE I attached below, either as to the individual compound itself or in a composition, or the process of making or the use thereof in methods according to the invention. In each of the compounds listed in TABLE I below, any hydrogen may be replaced by the substituent R^{X} which is a (C₁-C₆)alkyl, (C₂-C₆)alkenyl, (C₂-C₆)alkynyl, phenyl, (C₁-C₁₀)heteroaryl, (C₁-C₁₀)heterocyclic or (C₃-C₁₀)cycloalkyl substituent. Other embodiments of the invention are related to the specific subgenuses listed in TABLE I. In these subgenuses, any hydrogen can also be replaced by an R^{X} substituent.

TABLE I

| Compound | Chemical Structure | MF | MW |
|---|---|---|---|
| 1 | 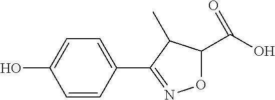 [SMILES: CC1C(c2ccc(O)cc2)=NOC1C(=O)O] | C11H11NO4 | 221.21 |
| 2 | 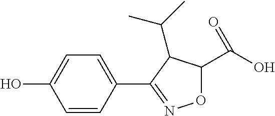 [SMILES: CC(C)C1C(c2ccc(O)cc2)=NOC1C(=O)O] | C13H15NO4 | 249.29 |
| 3 | 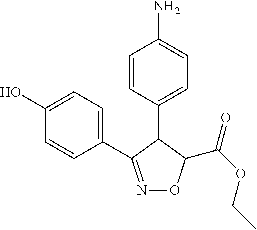 [SMILES: CCOC(=O)C1ON=C(c2ccc(O)cc2)C1c1ccc(N)cc1] | C18H18N2O4 | 326.36 |
| 4 | 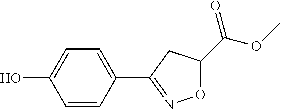 [SMILES: COC(=O)C1CC(c2ccc(O)cc2)=NO1] | C11H11NO4 | 221.21 |
| 5 | 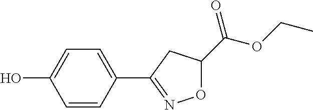 [SMILES: CCOC(=O)C1CC(c2ccc(O)cc2)=NO1] | C12H13NO4 | 235.24 |
| 6 | 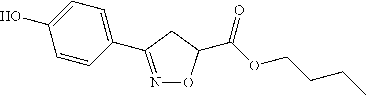 [SMILES: CCCCOC(=O)C1CC(c2ccc(O)cc2)=NO1] | C14H17NO4 | 263.3 |
| 7 | 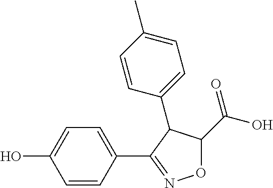 [SMILES: Cc1ccc(C2C(c3ccc(O)cc3)=NOC2C(=O)O)cc1] | C17H15NO4 | 297.31 |
| 8 | 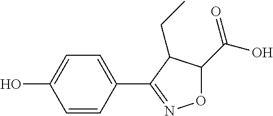 [SMILES: CCC1C(c2ccc(O)cc2)=NOC1C(=O)O] | C12H13NO4 | 235.24 |
| 9 | 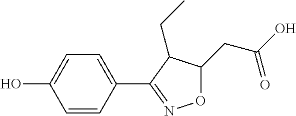 [SMILES: CCC1C(c2ccc(O)cc2)=NOC1CC(=O)O] | C13H15NO4 | 249.27 |
| 10 | 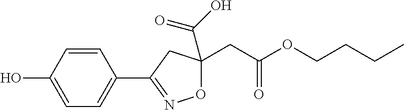 [SMILES: CCCCOC(=O)CC1(C(=O)O)CC(c2ccc(O)cc2)=NO1] | C16H19NO6 | 321.33 |
| 11 | 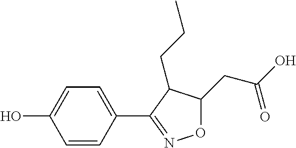 [SMILES: CCCC1C(c2ccc(O)cc2)=NOC1CC(=O)O] | C14H17NO4 | 263.3 |
| 12 | 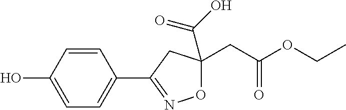 [SMILES: CCOC(=O)CC1(C(=O)O)CC(c2ccc(O)cc2)=NO1] | C14H15NO6 | 293.27 |
| 13 | 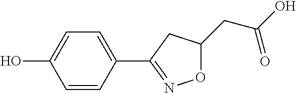 [SMILES: O=C(O)CC1CC(c2ccc(O)cc2)=NO1] | C11H11NO4 | 221.21 |
| 14 | 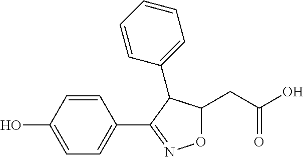 [SMILES: O=C(O)CC1ON=C(c2ccc(O)cc2)C1c1ccccc1] | C17H15NO4 | 297.31 |
| 15 | 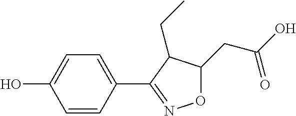 [SMILES: CCC1C(c2ccc(O)cc2)=NOC1CC(=O)O] | C13H15NO4 | 249.29 |
| 16 | 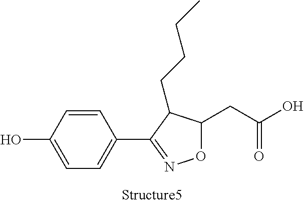 [SMILES: CCCCC1C(c2ccc(O)cc2)=NOC1CC(=O)O] | C15H19NO4 | 277.32 |
| 17 | 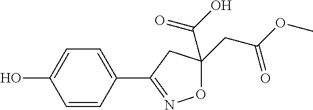 [SMILES: COC(=O)CC1(C(=O)O)CC(c2ccc(O)cc2)=NO1] | C13H13NO6 | 279.25 |
| 18 | 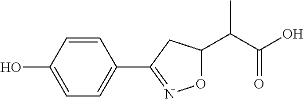 [SMILES: CC(C(=O)O)C1CC(c2ccc(O)cc2)=NO1] | C12H13NO4 | 235.24 |
| 19 | 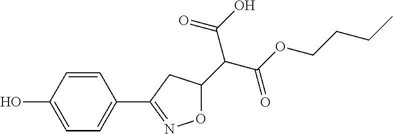 [SMILES: CCCCOC(=O)C(C(=O)O)C1CC(c2ccc(O)cc2)=NO1] | C16H19NO6 | 321.33 |
| 20 | 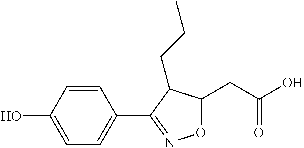 [SMILES: CCCC1C(c2ccc(O)cc2)=NOC1CC(=O)O] | C14H17NO4 | 263.29 |
| 21 | 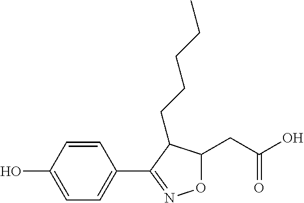 [SMILES: CCCCCC1C(c2ccc(O)cc2)=NOC1CC(=O)O] | C16H21NO4 | 291.34 |
| 22 | 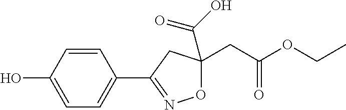 [SMILES: CCOC(=O)CC1(C(=O)O)CC(c2ccc(O)cc2)=NO1] | C14H15NO6 | 293.27 |
| 23 | 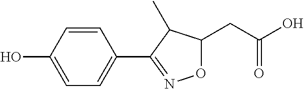 [SMILES: CC1C(c2ccc(O)cc2)=NOC1CC(=O)O] | C12H13NO4 | 235.24 |
| 24 | 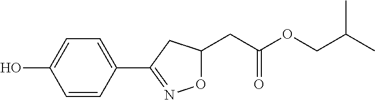 [SMILES: CC(C)COC(=O)CC1CC(c2ccc(O)cc2)=NO1] | C15H19NO4 | 277.32 |
| 25 | 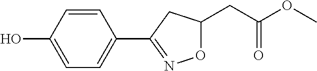 [SMILES: COC(=O)CC1CC(c2ccc(O)cc2)=NO1] | C12H13NO4 | 235.24 |
| 26 | 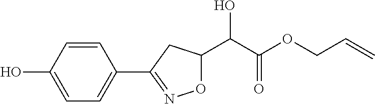 [SMILES: C=CCOC(=O)C(O)C1CC(c2ccc(O)cc2)=NO1] | C14H15NO5 | 277.27 |
| 27 | 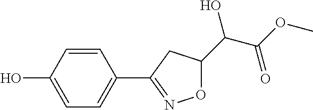 [SMILES: COC(=O)C(O)C1CC(c2ccc(O)cc2)=NO1] | C12H13NO5 | 251.24 |
| 28 | 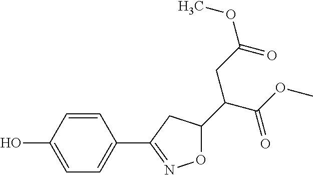 [SMILES: COC(=O)CC(C(=O)OC)C1CC(c2ccc(O)cc2)=NO1] | C15H17NO6 | 307.31 |
| 29 | 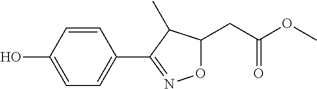 [SMILES: COC(=O)CC1ON=C(c2ccc(O)cc2)C1C] | C13H15NO4 | 249.26 |
| 30 | 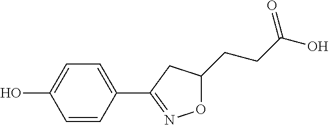 [SMILES: O=C(O)CCC1CC(c2ccc(O)cc2)=NO1] | C12H13NO4 | 235.24 |
| 31 | 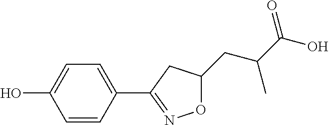 [SMILES: CC(CC1CC(c2ccc(O)cc2)=NO1)C(=O)O] | C13H15NO4 | 249.27 |
| 32 | 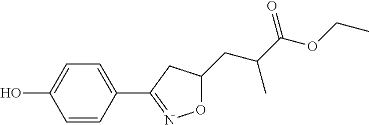 [SMILES: CCOC(=O)C(C)CC1CC(c2ccc(O)cc2)=NO1] | C15H19NO4 | 277.32 |
| 33 | 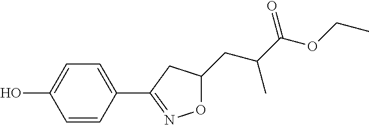 [SMILES: CCOC(=O)C(C)CC1CC(c2ccc(O)cc2)=NO1] | C14H17NO4 | 263.3 |
| 34 | 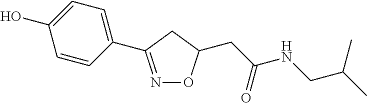 [SMILES: CC(C)CNC(=O)CC1CC(c2ccc(O)cc2)=NO1] | C15H20N2O3 | 276.33 |
| 35 | 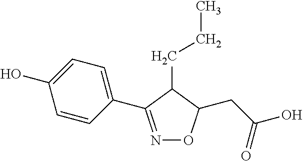 [SMILES: CCCC1C(c2ccc(O)cc2)=NOC1CC(=O)O] | C14H17NO4 | 263.29 |
| 36 | 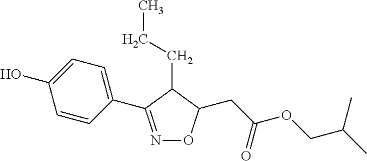 [SMILES: CCCC1C(c2ccc(O)cc2)=NOC1CC(=O)OCC(C)C] | C19H25NO4 | 319.4 |
| 37 | 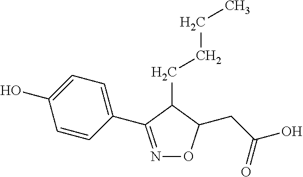 [SMILES: CCCCC1C(c2ccc(O)cc2)=NOC1CC(=O)O] | C15H19NO4 | 277.32 |
| 38 | 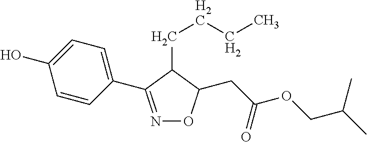 [SMILES: CCCCC1C(c2ccc(O)cc2)=NOC1CC(=O)OCC(C)C] | C19H27NO4 | 333.42 |
| 39 | 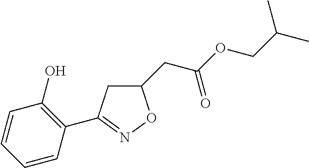 [SMILES: CC(C)COC(=O)CC1CC(c2ccccc2O)=NO1] | C15H19NO4 | 277.32 |
| 40 | 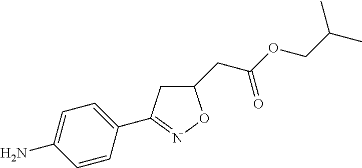 [SMILES: CC(C)COC(=O)CC1CC(c2ccc(N)cc2)=NO1] | C15H20N2O3 | 276.33 |
| 41 | 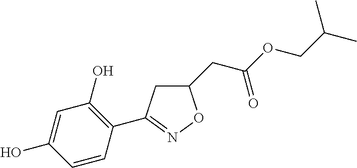 [SMILES: CC(C)COC(=O)CC1CC(c2ccc(O)cc2O)=NO1] | C15H19NO5 | 293.32 |
| 42 | 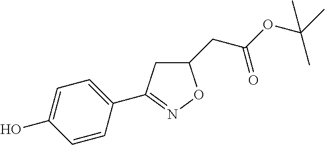 [SMILES: CC(C)(C)OC(=O)CC1CC(c2ccc(O)cc2)=NO1] | C15H19NO4 | 277.32 |
| 43 | 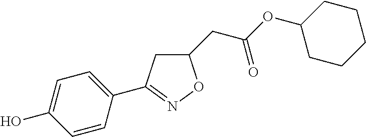 [SMILES: O=C(CC1CC(c2ccc(O)cc2)=NO1)OC1CCCCC1] | C17H21NO4 | 303.35 |
| 44 | 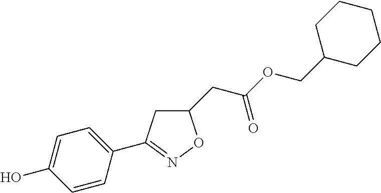 [SMILES: O=C(CC1CC(c2ccc(O)cc2)=NO1)OCC1CCCCC1] | C18H23NO4 | 317.38 |
| 45 | 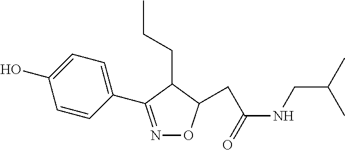 [SMILES: CCCC1C(c2ccc(O)cc2)=NOC1CC(=O)NCC(C)C] | C18H26N2O3 | 318.19 |
| 46 | 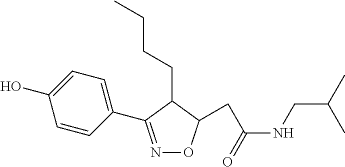 [SMILES: CCCCC1C(c2ccc(O)cc2)=NOC1CC(=O)NCC(C)C] | C19H28N2O3 | 332.21 |
| 47 | 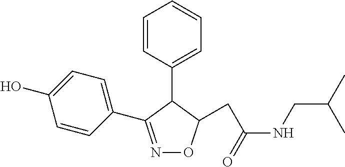 [SMILES: CC(C)CNC(=O)CC1ON=C(c2ccc(O)cc2)C1c1ccccc1] | C21H24N2O3 | 352.18 |
| 48 | 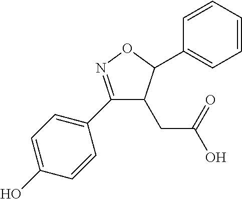 [SMILES: O=C(O)CC1C(c2ccc(O)cc2)=NOC1c1ccccc1] | C17H15NO4 | 297.31 |
| 49 | 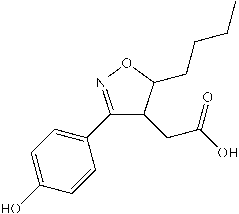 [SMILES: CCCCC1ON=C(c2ccc(O)cc2)C1CC(=O)O] | C15H19NO4 | 277.32 |
| 50 | 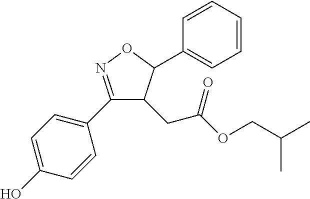 [SMILES: CC(C)COC(=O)CC1C(c2ccc(O)cc2)=NOC1c1ccccc1] | C21H24N2O3 | 353.18 |
| 51 | 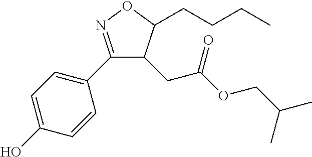 [SMILES: CCCCC1ON=C(c2ccc(O)cc2)C1CC(=O)OCC(C)C] | C19H28N2O3 | 333.42 |
| 52 | 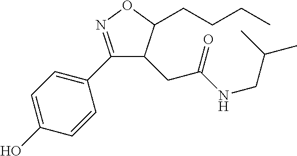 [SMILES: CCCCC1ON=C(c2ccc(O)cc2)C1CC(=O)NCC(C)C] | C19H28N2O3 | 332.21 |
| 53 | 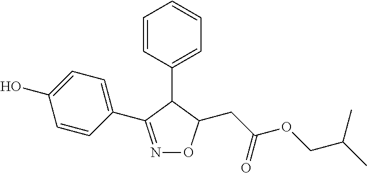 [SMILES: CC(C)COC(=O)CC1ON=C(c2ccc(O)cc2)C1c1ccccc1] | C21H23NO4 | 353.16 |
| 54 | 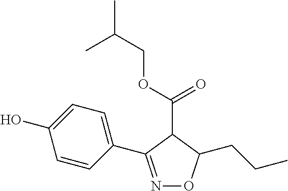 [SMILES: CCCC1ON=C(c2ccc(O)cc2)C1C(=O)OCC(C)C] | C17H23NO4 | 305.16 |
| 55 | 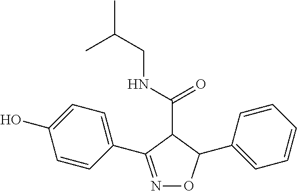 [SMILES: CC(C)CNC(=O)C1C(c2ccc(O)cc2)=NOC1c1ccccc1] | C20H22N2O3 | 338.16 |
| 56 | 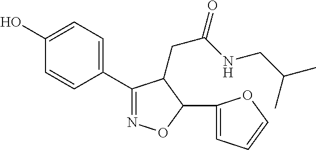 [SMILES: CC(C)CNC(=O)CC1C(c2ccc(O)cc2)=NOC1c1ccco1] | C19H22N2O4 | 342.16 |
| 57 | 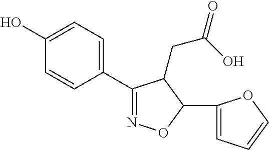 [SMILES: O=C(O)CC1C(c2ccc(O)cc2)=NOC1c1ccco1] | C15H13NO5 | 287.08 |
| 58 | 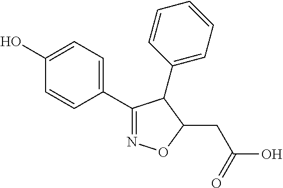 [SMILES: O=C(O)CC1ON=C(c2ccc(O)cc2)C1c1ccccc1] | C17H15NO4 | 297.31 |
| 59 | 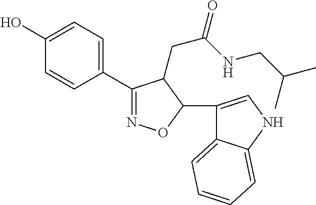 [SMILES: CC(C)CNC(=O)CC1C(c2ccc(O)cc2)=NOC1c1c[nH]c2ccccc12] | C23H25N3O3 | 391.46 |
| 60 | 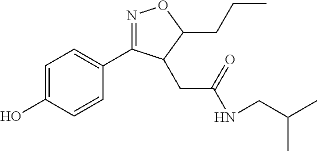 [SMILES: CCCC1ON=C(c2ccc(O)cc2)C1CC(=O)NCC(C)C] | C18H28N2O3 | 318.41 |
| 61 | 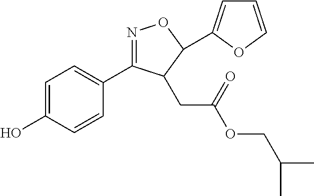 [SMILES: CC(C)COC(=O)CC1C(c2ccc(O)cc2)=NOC1c1ccco1] | C19H21NO5 | 343.37 |
| 62 | 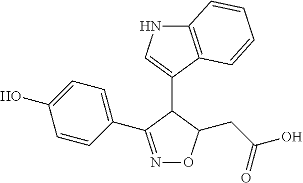 [SMILES: O=C(O)CC1ON=C(c2ccc(O)cc2)C1c1c[nH]c2ccccc12] | C19H16N2O4 | 336.34 |
| 63 | 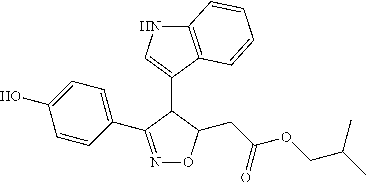 [SMILES: CC(C)COC(=O)CC1ON=C(c2ccc(O)cc2)C1c1c[nH]c2ccccc12] | C23H24N2O4 | 392.45 |
| 64 | 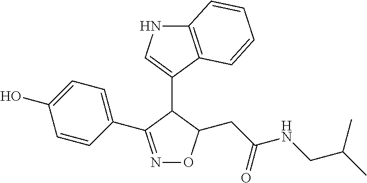 [SMILES: CC(C)CNC(=O)CC1ON=C(c2ccc(O)cc2)C1c1c[nH]c2ccccc12] | C23H25N3O3 | 391.46 |
| 65 | 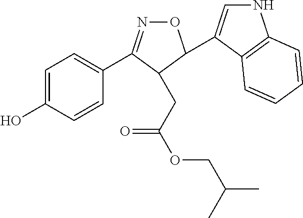 [SMILES: CC(C)COC(=O)CC1C(c2ccc(O)cc2)=NOC1c1c[nH]c2ccccc12] | C23H24N2O4 | 392.45 |
| 66 | 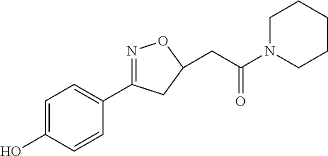 [SMILES: O=C(CC1CC(c2ccc(O)cc2)=NO1)N1CCCCC1] | C16H29N2O3 | 288.34 |
| 67 | 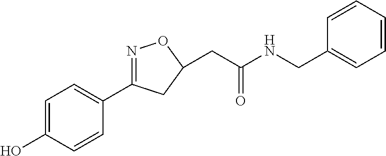 [SMILES: O=C(CC1CC(c2ccc(O)cc2)=NO1)NCc1ccccc1] | C18H18N2O3 | 310.35 |
| 68 | 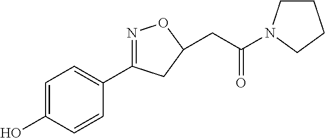 [SMILES: O=C(CC1CC(c2ccc(O)cc2)=NO1)N1CCCC1] | C15H18N2O3 | 274.32 |
| 69 | 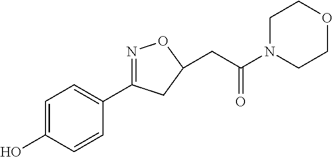 [SMILES: O=C(CC1CC(c2ccc(O)cc2)=NO1)N1CCOCC1] | C15H18N2O4 | 290.31 |
| 70 | 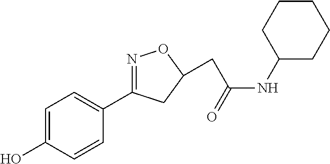 [SMILES: O=C(CC1CC(c2ccc(O)cc2)=NO1)NC1CCCCC1] | C17H22N2O3 | 302.16 |
| 71 | 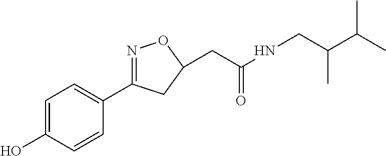 [SMILES: CC(C)C(C)CNC(=O)CC1CC(c2ccc(O)cc2)=NO1] | C17H24N2O3 | 304.38 |
| 72 | 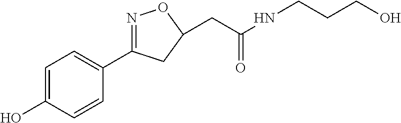 [SMILES: O=C(CC1CC(c2ccc(O)cc2)=NO1)NCCCO] | C14H18N2O4 | 278.3 |
| 73 | 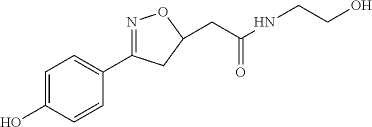 [SMILES: O=C(CC1CC(c2ccc(O)cc2)=NO1)NCCO] | C13H16N2O4 | 264.28 |
| 74 | 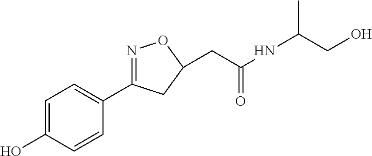 [SMILES: CC(CO)NC(=O)CC1CC(c2ccc(O)cc2)=NO1] | C14H18N2O4 | 278.3 |
| 75 | 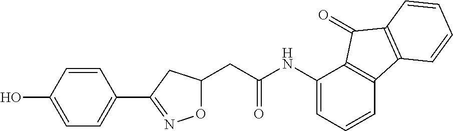 [SMILES: O=C(CC1CC(c2ccc(O)cc2)=NO1)Nc1cccc2c1C(=O)c1ccccc1-2] | C24H18N2O4 | 398.41 |
| 76 | 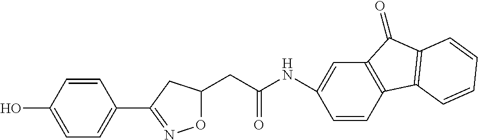 [SMILES: O=C(CC1CC(c2ccc(O)cc2)=NO1)Nc1ccc2c(c1)C(=O)c1ccccc1-2] | C24H18N2O4 | 398.41 |
| 77 | 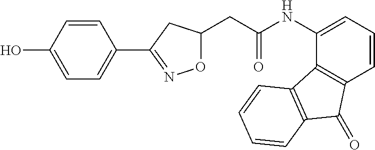 [SMILES: O=C(CC1CC(c2ccc(O)cc2)=NO1)Nc1cccc2c1-c1ccccc1C2=O] | C24H18N2O4 | 398.41 |
| 78 | 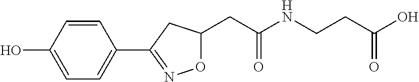 [SMILES: O=C(O)CCNC(=O)CC1CC(c2ccc(O)cc2)=NO1] | C14H16N2O5 | 292.29 |
| 79 | 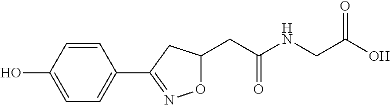 [SMILES: O=C(O)CNC(=O)CC1CC(c2ccc(O)cc2)=NO1] | C13H14N2O5 | 278.26 |
| 80 | 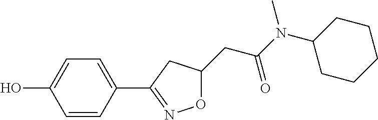 [SMILES: CN(C(=O)CC1CC(c2ccc(O)cc2)=NO1)C1CCCCC1] | C18H24N2O3 | 316.39 |
| 81 | 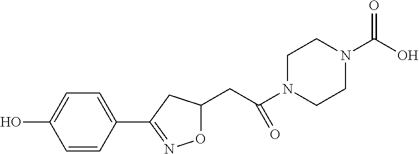 [SMILES: O=C(O)N1CCN(C(=O)CC2CC(c3ccc(O)cc3)=NO2)CC1] | C16H19N3O5 | |
| 82 | 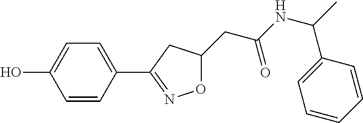 [SMILES: CC(NC(=O)CC1CC(c2ccc(O)cc2)=NO1)c1ccccc1] | C19H20N2O3 | 324.37 |
| 83 | 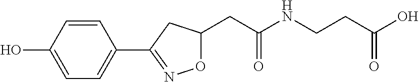 [SMILES: O=C(O)CCNC(=O)CC1CC(c2ccc(O)cc2)=NO1] | C14H16N2O5 | 292.29 |
| 84 | 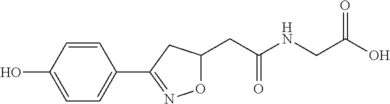 [SMILES: O=C(O)CNC(=O)CC1CC(c2ccc(O)cc2)=NO1] | C13H14N2O5 | 278.26 |
| 85 | 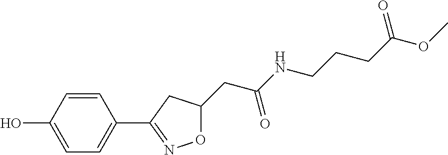 [SMILES: COC(=O)CCCNC(=O)CC1CC(c2ccc(O)cc2)=NO1] | C16H21N2O5 | 321.35 |
| 86 | 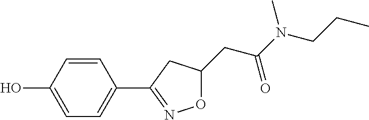 [SMILES: CCCN(C)C(=O)CC1CC(c2ccc(O)cc2)=NO1] | C15H21N2O3 | 277.34 |
| Subgenus A | 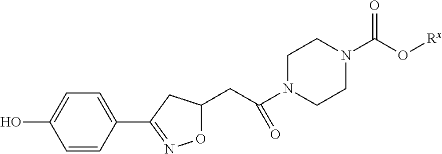 | variable | |
| Subgenus B | 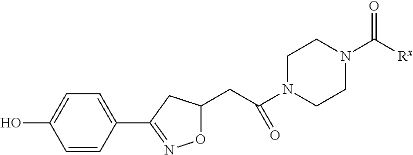 | variable | |
| Subgenus C | 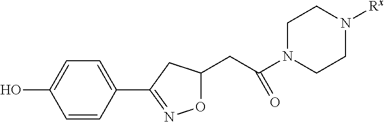 | variable | |
| 87 | 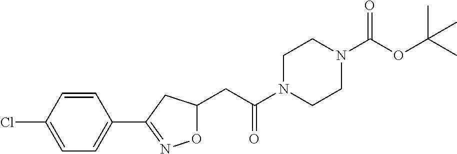 [SMILES: CC(C)(C)OC(=O)N1CCN(C(=O)CC2CC(c3ccc(Cl)cc3)=NO2)CC1] | C20H26N3O4Cl | 408 |
| 88 | 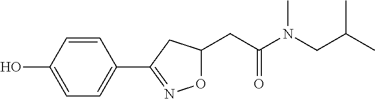 [SMILES: CC(C)CN(C)C(=O)CC1CC(c2ccc(O)cc2)=NO1] | C16H21N2O3 | 290 |
| 89 | 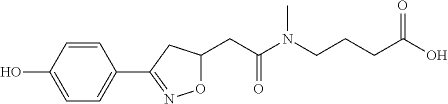 [SMILES: CN(CCCC(=O)O)C(=O)CC1CC(c2ccc(O)cc2)=NO1] | C16H20N2O5 | 321 |
| 90 | 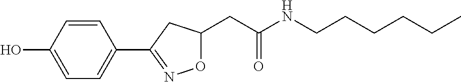 [SMILES: CCCCCCNC(=O)CC1CC(c2ccc(O)cc2)=NO1] | C18H26N2O3 | 319 |
| 91 | 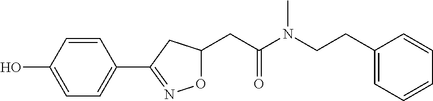 [SMILES: CN(CCc1ccccc1)C(=O)CC1CC(c2ccc(O)cc2)=NO1] | C20H22N2O3 | 339 |
| 92 | 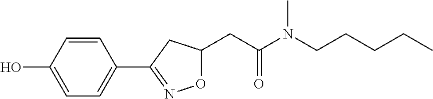 [SMILES: CCCCCN(C)C(=O)CC1CC(c2ccc(O)cc2)=NO1] | C17H24N2O3 | 305 |
| 93 | 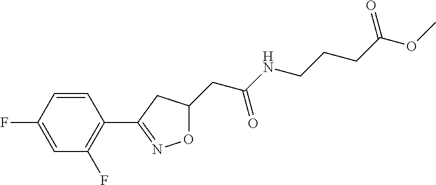 [SMILES: COC(=O)CCCNC(=O)CC1CC(c2ccc(F)cc2F)=NO1] | C16H18N2O4F2 | 341 |
| 94 | 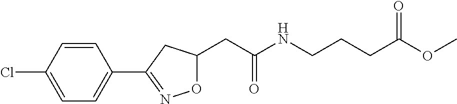 [SMILES: COC(=O)CCCNC(=O)CC1CC(c2ccc(Cl)cc2)=NO1] | C16H19N2O4Cl | 339 |
| 95 | 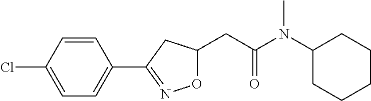 [SMILES: CN(C(=O)CC1CC(c2ccc(Cl)cc2)=NO1)C1CCCCC1] | C18H26N2O2Cl | 335 |
| 96 | 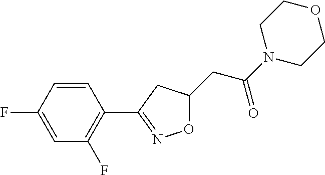 [SMILES: O=C(CC1CC(c2ccc(F)cc2F)=NO1)N1CCOCC1] | C15H16N2O3F2 | 311 |
| 97 | 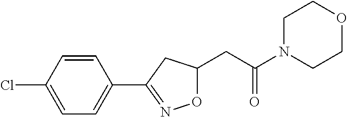 [SMILES: O=C(CC1CC(c2ccc(Cl)cc2)=NO1)N1CCOCC1] | C15H17N2O3Cl | 309 |
| 98 | 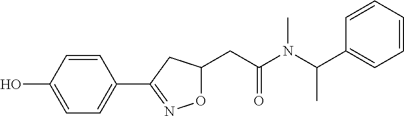 | C26H22N2O3 | 339 |
| 99 | 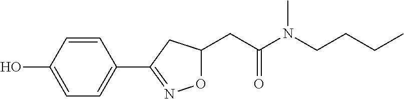 [SMILES: CCCCN(C)C(=O)CC1CC(c2ccc(O)cc2)=NO1] | C16H23N2O3 | 292 |
| 100 | 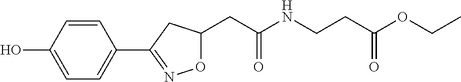 [SMILES: CCOC(=O)CCNC(=O)CC1CC(c2ccc(O)cc2)=NO1] | C16H19N2O5 | 320 |
| 101 | 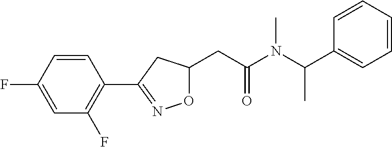 [SMILES: CC(c1ccccc1)N(C)C(=O)CC1CC(c2ccc(F)cc2F)=NO1] | C20H20N2O2F2 | 359 |
| 102 | 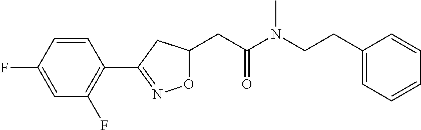 [SMILES: CN(CCc1ccccc1)C(=O)CC1CC(c2ccc(F)cc2F)=NO1] | C20H20N2O2F2 | 359 |
| 103 | 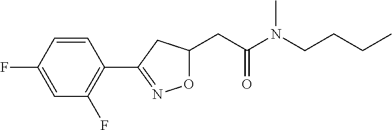 [SMILES: CCCCN(C)C(=O)CC1CC(c2ccc(F)cc2F)=NO1] | C16H20N2O2F2 | 311 |
| 104 | 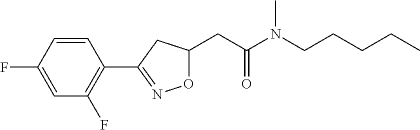 [SMILES: CCCCCN(C)C(=O)CC1CC(c2ccc(F)cc2F)=NO1] | C17H22N2O2F2 | 324.37 |
| 105 | 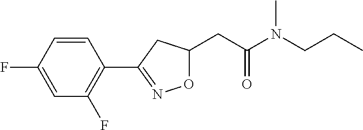 [SMILES: CCCN(C)C(=O)CC1CC(c2ccc(F)cc2F)=NO1] | C15H18N2O2F2 | 296.31 |
| 106 | 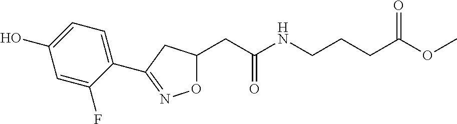 [SMILES: COC(=O)CCCNC(=O)CC1CC(c2ccc(O)cc2F)=NO1] | C19H18N2O5F | 338 |
| 107 | 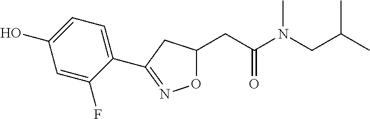 [SMILES: CC(C)CN(C)C(=O)CC1CC(c2ccc(O)cc2F)=NO1] | C16H21N2O3F | 309 |
| 108 | 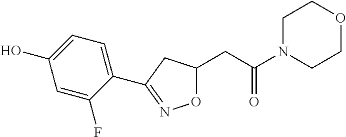 [SMILES: O=C(CC1CC(c2ccc(O)cc2F)=NO1)N1CCOCC1] | C15H17N2O4F | 309 |
| 109 | 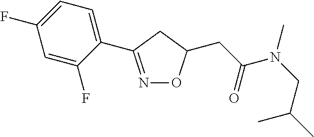 [SMILES: CC(C)CN(C)C(=O)CC1CC(c2ccc(F)cc2F)=NO1] | C16H20N2O2F2 | 311 |
| 110 | 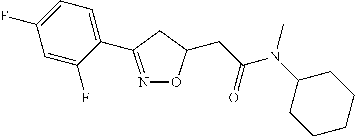 [SMILES: CN(C(=O)CC1CC(c2ccc(F)cc2F)=NO1)C1CCCCC1] | C18H22N2O2F2 | 337 |
| 111 | 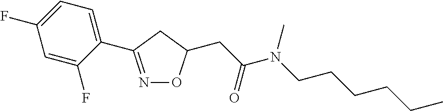 [SMILES: CCCCCCN(C)C(=O)CC1CC(c2ccc(F)cc2F)=NO1] | C18H24N2O2F2 | 339 |
| 112 | 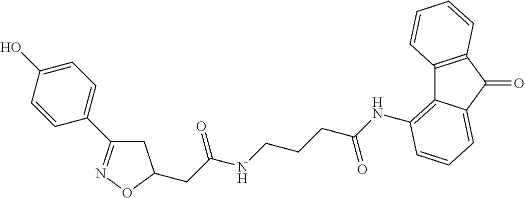 [SMILES: O=C(CC1CC(c2ccc(O)cc2)=NO1)NCCCC(=O)Nc1cccc2c1-c1ccccc1C2=O] | C28H25N3O5 | 484 |
| 113 | 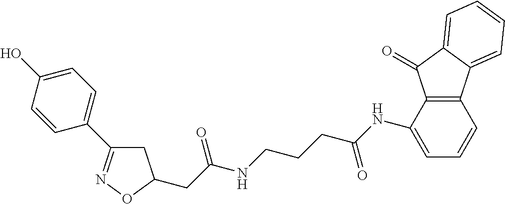 [SMILES: O=C(CC1CC(c2ccc(O)cc2)=NO1)NCCCC(=O)Nc1cccc2c1C(=O)c1ccccc1-2] | C28H25N3O5 | 484 |
| 114 | 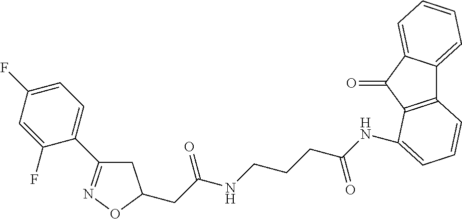 [SMILES: O=C(CC1CC(c2ccc(F)cc2F)=NO1)NCCCC(=O)Nc1cccc2c1C(=O)c1ccccc1-2] | C28H23N3O4F2 | 504 |
| 115 | 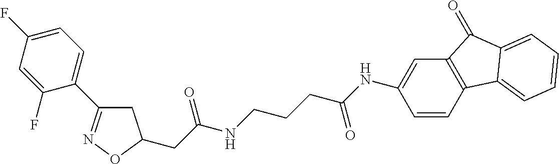 [SMILES: O=C(CC1CC(c2ccc(F)cc2F)=NO1)NCCCC(=O)Nc1ccc2c(c1)C(=O)c1ccccc1-2] | C28H23N3O4F2 | 504 |
| 116 | 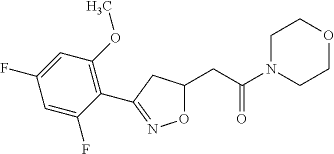 [SMILES: COc1cc(F)cc(F)c1C1=NOC(CC(=O)N2CCOCC2)C1] | C16H18N2O4F2 | 341 |
| 117 | 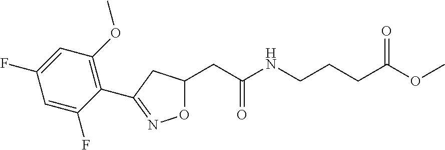 [SMILES: COC(=O)CCCNC(=O)CC1CC(c2c(F)cc(F)cc2OC)=NO1] | C17H20N2O5f2 | 371 |
| 118 | 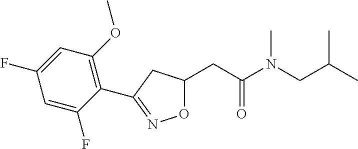 [SMILES: COc1cc(F)cc(F)c1C1=NOC(CC(=O)N(C)CC(C)C)C1] | C17H22N2O3F2 | 341 |
| 119 | 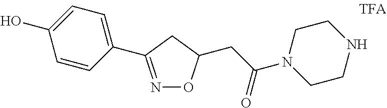 [SMILES: O=C(CC1CC(c2ccc(O)cc2)=NO1)N1CCNCC1] | C14H19O3N3TFA | 392 |
| 120 | 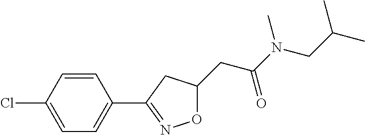 [SMILES: CC(C)CN(C)C(=O)CC1CC(c2ccc(Cl)cc2)=NO1] | C16H21N2O2Cl | 309 |
| 121 | 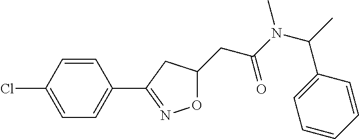 [SMILES: CC(c1ccccc1)N(C)C(=O)CC1CC(c2ccc(Cl)cc2)=NO1] | C20H21N2O2Cl | 357 |
| 122 | 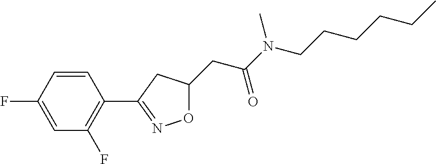 [SMILES: CCCCCCN(C)C(=O)CC1CC(c2ccc(F)cc2F)=NO1] | C18H24F2N2O2 | 338.4 |
| 123 | 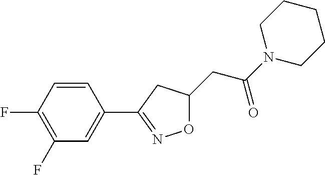 [SMILES: O=C(CC1CC(c2ccc(F)c(F)c2)=NO1)N1CCCCC1] | C16H18F2N2O2 | 308.32 |
| 124 | 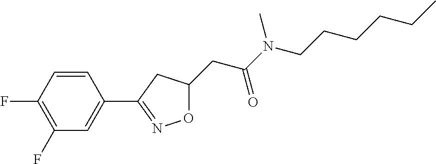 [SMILES: CCCCCCN(C)C(=O)CC1CC(c2ccc(F)c(F)c2)=NO1] | C18H24F2N2O2 | 338.4 |
| 125 | 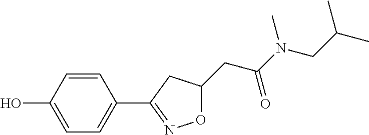 [SMILES: CC(C)CN(C)C(=O)CC1CC(c2ccc(O)cc2)=NO1] | C16H22N2O3 | 290.37 |
| 126 | 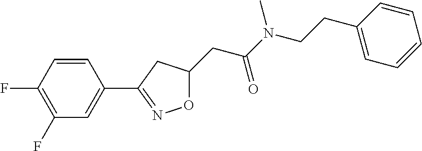 [SMILES: CN(CCc1ccccc1)C(=O)CC1CC(c2ccc(F)c(F)c2)=NO1] | C20H20F2N2O2 | 358.38 |
| 127 | 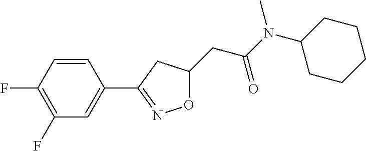 [SMILES: CN(C(=O)CC1CC(c2ccc(F)c(F)c2)=NO1)C1CCCCC1] | C18H22F2N2O2 | 336.38 |
| 128 | 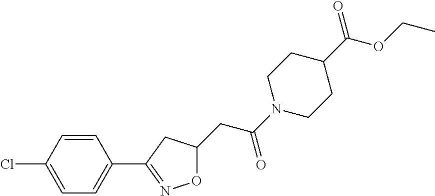 [SMILES: CCOC(=O)C1CCN(C(=O)CC2CC(c3ccc(Cl)cc3)=NO2)CC1] | C19H23ClN2O4 | 378.85 |

The compounds of the present invention have utility in pharmacological compositions for the treatment and prevention of many diseases and disorders characterized by a MIF response, whereby MIF is released from cellular sources and MIF production is enhanced. A compound of the invention can be administered to a human patient by itself or in pharmaceutical compositions where it is mixed with suitable carriers or excipients at doses to treat or ameliorate various conditions characterized by MIF release. A therapeutically effective dose may refer to that amount of the compound sufficient to inhibit MIF tautomerase activity and MIF bioactivity, it being understood that such inhibition may occur at different concentrations such that a person skilled in the art could determine the required dosage of compound to inhibit the target MIF activity. Therapeutically effective doses may be administered alone or as adjunctive therapy in combination with other treatments, such as steroidal or non-steroidal anti-inflammatory agents, or anti-tumor agents. Techniques for the formulation and administration of the compounds of the instant application may be found in Remington's Pharmaceutical Sciences. Mack Publishing Co., Easton, Pa., latest addition.

Suitable routes of administration may, for example, include oral, rectal, transmucosal, buccal, intravaginal, or intestinal administration; parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. Furthermore, one may administer a compound of the present invention in a targeted drug delivery system, for example in a liposome.

The pharmaceutical compositions and compounds of the present invention may be manufactured in a manner that is itself known, e.g., by means of conventional mixing, dissolving, dragee-making, levitating, emulsifying, encapsulating, entrapping, or lyophilizing processes. Pharmaceutical compositions for use in accordance with the present invention thus may be formulated in conventional manner using one or more physiologically acceptable carriers comprising excipients and auxiliaries that facilitate processing of the active compounds into preparations, which can be used pharmaceutically. Proper formulation is dependent upon the route of administration chosen.

Any combination of one or more compounds of Formulas I, II, salts, prodrugs, metabolites, isotopically-labeled compounds, tautomers, isomers, and/or atropisomers is possible in the composition.

For injection, the compounds of the invention may be formulated in aqueous solutions, preferably in physiologically compatible buffers, such as Hank's solution, Ringer's solution, or physiological saline buffer. For transmucosal administration, penetrants appropriate to the barrier to be permeated are used in the formulation. Such penetrants are known in the art.

For oral administration, the compounds can be formulated readily by combining the active compounds with pharmaceutically acceptable carriers well known to those in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the compound with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as polyionic block (co)polymer, sodium carboxymethyl cellulose, sorbitol, or dextran. Optionally, the suspension may also contain suitable stabilizers or agents which increase the solubility of the compounds to allow for the preparation of highly concentrated solutions, e.g., polyionic block (co)polymers.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various forms of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention identified as inhibitors of MIF activity may be provided as salts with pharmaceutically compatible counterions. Pharmaceutically compatible salts may be formed with many acids, including but not limited to hydrochloric, sulfuric, acetic, lactic, tartaric, malic, succinic, etc.; or bases. Salts tend to be more soluble in aqueous or other protonic solvents than are the corresponding free base forms. Examples of pharmaceutically acceptable salts, carriers or excipients are well known to those skilled in the art and can be found, for example, in Remington's Pharmaceutical Sciences, 18th Edition, A. R. Gennaro, Ed., Mack Publishing Co., Easton, Pa. (1990). Such salts include, but are not limited to, sodium, potassium, lithium, calcium, magnesium, iron, zinc, hydrochloride, hydrobromide, hydroiodide, acetate, citrate, tartrate and maleate salts, and the like.

Pharmaceutical compositions suitable for use in the present invention include compositions wherein the active ingredients are contained in an effective amount to achieve their intended purpose. More specifically, a therapeutically effective amount means an amount effective to prevent or inhibit development or progression of a disease characterized by MIF release and production in the subject being treated. Determination of the effective amounts is well within the capability of those skilled in the art, especially in light of the detailed disclosure provided herein.

For any compound used in the method of the invention, the therapeutically effective dose can be estimated initially from tautomerase inhibition assays and cell culture assays. Such information can be used to more accurately determine useful doses in humans. Toxicity and therapeutic efficacy of such compounds can be determined by standard pharmaceutical, pharmacological, and toxicological procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices (ED₅₀>LD₅₀ or ED₅₀>>LD₅₀) are preferred. The data obtained from cell culture assays or animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl, et al. (1975), in The Pharmacological Basis of Therapeutics, Chapter. 1 page 1).

Dosage amount and interval may be adjusted individually to provide plasma levels of the active moiety which are sufficient to maintain the desired modulating effects, or minimal effective concentration (MEC). The MEC will vary for each compound but can be estimated from in vitro data; e.g., the concentration necessary to achieve a 50-90% inhibition of MIF activity. Dosages necessary to achieve the MEC will depend on individual characteristics and route of administration. However, HPLC assays, bioassays or immunoassays can be used to determine plasma concentrations.

Dosage intervals can also be determined using the MEC value. Compounds should be administered using a regimen that maintains plasma levels above the MEC for 1-90% of the time, preferably between 30-90% and most preferably between 50-90%. These ranges include 1, 2, 3, 4, 5, 6, 7, 8, 9, 10, 20, 30, 40, 50, 60, 70, 80, 90, 99, and any combination thereof.

The active ingredient may be present in a pharmaceutical composition in an amount ranging from 0.1 to 99.9% by weight. These ranges include 0.1, 0.5, 0.9, 1, 2, 3, 4, 5, 6, 7, 8, 9, 10, 20, 30, 40, 50, 60, 70, 80, 90, 99, 99.5, 99.9% by weight and any combination thereof.

In cases of local administration for instance, direct introduction into a target organ or tissue, or selective uptake, the effective local concentration of the drug may not be related to plasma concentration.

The amount of composition administered will, of course, be dependent on the subject being treated, on the subject's weight, on the subject's age, on the severity of the affliction, on the manner of administration, and on the judgment of the prescribing physician.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

The compounds of Formulas I or II, or a pharmaceutically acceptable salt thereof can be used in the manufacture of a medicament for the prophylactic or therapeutic treatment of any disease state in a human, or other mammal, which is exacerbated or caused by excessive or unregulated cytokine production by such mammal's cells, such as but not limited to monocytes and/or macrophages.

The enzyme activity (tautomerase) of MIF and the substrates it accepts provide an enzymatic activity assay for designing low molecular weight agents that bind to MIF and disrupt its biological activity. The present invention provides methods of use for the compounds in a genus of such compounds having isoxazoline structures.

The present invention further provides a pharmaceutical composition comprising the isoxazoline compound, or a pharmaceutically acceptable salt thereof, and a pharmaceutically acceptable carrier or diluant, wherein the composition comprises an effective amount of the compound of the above formula.

The present invention also provides a pharmaceutical composition comprising a compound having an isoxazoline or isoxazoline-related moiety, and a pharmaceutically acceptable carrier, wherein the compound forms a stable interaction with at least one amino acid residue of a MIF protein.

The present invention provides a method for treating inflammatory disorders (including, but not limited to, arthritis, proliferative vascular disease, ARDS (acute respiratory distress syndrome), cytokine-mediated toxicity, sepsis, septic shock, psoriasis, interleukin-2 toxicity, asthma, MIF-mediated conditions, autoimmune disorders (including but not limited to rheumatoid arthritis, insulin-dependent diabetes, multiple sclerosis, graft versus host disease, lupus syndromes), tumor growth or angiogenesis, or any condition characterized by local or systemic MIF release or synthesis, comprising administering an effective amount of a compound having an isoxazoline moiety, wherein the compound forms an interaction with MIF protein. For example, the compound may bind to MIF protein, thereby interfering with the biological and/or enzymatic activity of MIF protein. The binding may be reversible or irreversible.

In accordance with the activity of MIF to interfere with the anti-inflammatory effects of steroids (such as the anti-inflammatory glucocorticoids), the compounds of Formula I or II find further utility to enhance the activity and therapeutic benefits of both endogenously arising and exogenously administered steroidal anti-inflammatory agents. Such benefits may, in some cases, be most evident by a reduced need for steroid therapy (e.g., lower dose amount or frequency; less potent agent; reduced need for systemic administration) or by reduced side-effects associated with steroid administration. The benefits of administering a MIF inhibitor (and specifically a compound of Formula I or II) may be realized as a monotherapy, using only the MIF inhibitor of the present invention, or as a combination therapy with additional anti-inflammatory agents, including especially, but without limitation, an anti-inflammatory steroid. Such combination therapy may be achieved through administration of a single formulation or pharmaceutical composition that combines the MIF inhibitor (particularly an inhibitor of Formula I or II) with at least one other anti-inflammatory agent (which may be a steroidal or a non-steroidal anti-inflammatory agent), or through administration of separate formulations or pharmaceutical compositions in conjunction with each other, or both.

Compounds of Formulas I and II are also capable of inhibiting pro-inflammatory cytokines affected by MIF, such as IL-1, IL-2, IL-6, IL-8, IFN-γ and TNF, and are therefore of use in therapy. IL-1, IL-2, IL-6, IL-8, IFN-γ and TNF affect a wide variety of cells and tissues and these cytokines, as well as other leukocyte-derived cytokines, are important and critical inflammatory mediators of a wide variety of disease states and conditions. The inhibition of these cytokines is of benefit in controlling, reducing and alleviating many of these disease states.

Accordingly, the present invention provides a method of treating a cytokine mediated disease which comprises administering an effective cytokine-interfering amount of a compound of Formula I or II or a pharmaceutically acceptable salt thereof.

In particular, compounds of Formulas I or II or a pharmaceutically acceptable salt thereof are of use in the therapy of any disease state in a human, or other mammal, which is exacerbated by or caused by excessive or unregulated MIF, IL-1, IL-2, IL-6, IL-8, IFN-γ and TNF production by such mammal's cells, such as, but not limited to, monocytes and/or macrophages.

Accordingly, in another aspect, this invention relates to a method of inhibiting the production of IL-1 in a mammal in need thereof which comprises administering to said mammal an effective amount of a compound of Formula I or II a pharmaceutically acceptable salt thereof. There are many disease states in which excessive or unregulated IL-1 production is implicated in exacerbating and/or causing the disease. These include rheumatoid arthritis, osteoarthritis, meningitis, ischemic and hemorrhagic stroke, neurotrauma/closed head injury, stroke, endotoxemia and/or toxic shock syndrome, other acute or chronic inflammatory disease states such as the inflammatory reaction induced by endotoxin or inflammatory bowel disease, tuberculosis, atherosclerosis, muscle degeneration, multiple sclerosis, cachexia, bone resorption, psoriatic arthritis, Reiter's syndrome, rheumatoid arthritis, gout, traumatic arthritis, rubella arthritis and acute synovitis. Recent evidence also links IL-1 activity to diabetes, pancreatic cells disease, and Alzheimer's disease.

In a further aspect, this invention relates to a method of inhibiting the production of TNF in a mammal in need thereof which comprises administering to said mammal an effective amount of a compound of Formula I or II or a pharmaceutically acceptable salt thereof. Excessive or unregulated TNF production has been implicated in mediating or exacerbating a number of diseases including rheumatoid arthritis, rheumatoid spondylitis, osteoarthritis, gouty arthritis and other arthritic conditions, sepsis, septic shock, endotoxic shock, gram negative sepsis, toxic shock syndrome, adult respiratory distress syndrome, stroke, cerebral malaria, chronic obstructive pulmonary disease, chronic pulmonary inflammatory disease, silicosis, pulmonary sarcoidosis, bone resorption diseases, such as osteoporosis, cardiac, brain and renal reperfusion injury, graft vs. host reaction, allograft rejections, fever and myalgias due to infection, such as influenza (including HIV-induced forms), cerebral malaria, meningitis, ischemic and hemorrhagic stroke, cachexia secondary to infection or malignancy, cachexia secondary to acquired immune deficiency syndrome (AIDS), AIDS, ARC (AIDS related complex), keloid formation, scar tissue formation, inflammatory bowel disease, Crohn's disease, ulcerative colitis and pyresis.

Compounds of Formula I or II are also useful in the treatment of viral infections, where such viruses are sensitive to upregulation by TNF or will elicit TNF production in vivo. The viruses contemplated for treatment herein are those that produce TNF as a result of infection, or those which are sensitive to inhibition, such as by decreased replication, directly or indirectly, by the TNF inhibiting-compounds of Formula I or II. Such viruses include, but are not limited to HIV-1, HIV-2 and HIV-3, Cytomegalovirus (CMV), Influenza, adenovirus and the Herpes group of viruses, such as but not limited to, Herpes Zoster and Herpes Simplex. Accordingly, in a further aspect, this invention relates to a method of treating a mammal afflicted with a human immunodeficiency virus (HIV) which comprises administering to such mammal an effective TNF inhibiting amount of a compound of Formula I or II or a pharmaceutically acceptable salt thereof.

Compounds of Formula I or II may also be used in association with the veterinary treatment of mammals, other than in humans, in need of inhibition of TNF production. TNF mediated diseases for treatment, in animals include disease states such as those noted above, but in particular viral infections. Examples of such viruses include, but are not limited to, lentivirus infections such as, equine infectious anaemia virus, caprine arthritis virus, visna virus, or maedi virus or retrovirus infections, such as but not limited to feline immunodeficiency virus (FIV), bovine immunodeficiency virus, or canine immunodeficiency virus or other retroviral infections.

The compounds of Formula I or II may also be used topically in the treatment of topical disease states mediated by or exacerbated by excessive cytokine production, such as by IL-1 or TNF respectively, such as inflamed joints, eczema, contact dermatitis psoriasis and other inflammatory skin conditions such as sunburn; inflammatory eye conditions including conjunctivitis; pyresis, pain and other conditions associated with inflammation. Periodontal disease has also been implemented in cytokine production, both topically and systemically. Hence, the use of compounds of Formula I or II to control the inflammation associated with cytokine production in such peroral diseases such as gingivitis and periodontitis is another aspect of the present invention.

Compounds of Formula I or II have also been shown to inhibit the production of IL-8 (Interleukin-8, NAP). Accordingly, in a further aspect, this invention relates to a method of inhibiting the production of IL-8 in a mammal in need thereof which comprises administering, to said mammal an effective amount of a compound of Formula I or II or a pharmaceutically acceptable salt thereof.

There are many disease states in which excessive or unregulated IL-8 production is implicated in exacerbating and/or causing the disease. These diseases are characterized by massive neutrophil infiltration such as, psoriasis, inflammatory bowel disease, asthma, cardiac and renal reperfusion injury, adult respiratory distress syndrome, thrombosis and glomerulonephritis. All of these diseases are associated with increased LL-8 production which is responsible for the chemotaxis of neutrophils into the inflammatory site. In contrast to other inflammatory cytokines (IL-1, TNF, and IL-6), IL-8 has the unique property of promoting neutrophil chemotaxis and activation. Therefore, the inhibition of IL-8 production would lead to a direct reduction in the neutrophil infiltration.

The compounds of Formula I or II are administered in an amount sufficient to inhibit a cytokine, in particular MIF, IL-1, IL-2, IL-6, IL-8, IFN-γ and TNF, production such that it is regulated down to normal levels, or in some case to subnormal levels, so as to ameliorate or prevent the disease state. Abnormal levels of MIF, IL-1, IL-2, IL-6, IL-8, IFN-γ and TNF, for instance in the context of the present invention, constitute: (i) levels of free (not cell bound) MIF, IL-1, IL-2, IL-6, IL-8, IFN-γ and TNF greater than or equal to 1 picogram per ml; (ii) any cell associated MIF, IL-1, IL-2, IL-6, IL-8, IFN-γ and TNF; or (iii) the presence of MIF, IL-1, IL-2, IL-6, IL-8, IFN-γ and TNF mRNA above basal levels in cells or tissues in which MIF, IL-1, IL-2, IL-6, IL-8, IFN-γ and TNF, respectively, is produced.

As used herein, the term “inhibiting the production of MIF, IL-1, IL-2, IL-6, IL-8, IFN-γ and TNF” refers to:

a) a decrease of excessive in vivo levels of the cytokine MIF, IL-1, IL-2, IL-6, IL-8, IFN-γ and TNF in a human to normal or sub-normal levels by inhibition of the in vivo release of the cytokine by all or select cells, including but not limited to monocytes or macrophages;

b) a down regulation, at the transcription level, of excessive in vivo levels of the cytokine MIF, IL-1, IL-2, IL-6, IL-8, IFN-γ and TNF in a human to normal or sub-normal levels;

c) a down regulation, at the post-transcription level, of excessive in vivo levels of the cytokine MIF, IL-1, IL-2, IL-6, IL-8, IFN-γ and TNF in a human to normal or sub-normal levels;

d) a down regulation, by inhibition of the direct synthesis of the cytokine MIF, IL-1, IL-2, IL-6, IL-8, IFN-γ and TNF as a postranslational event to normal or sub-normal levels; or

e) a down regulation, at the translational level, of excessive in vivo levels of the cytokine MIF, IL-1, IL-2, IL-6, IL-8, IFN-γ and TNF in a human to normal or sub-normal levels.

As used herein, the term “MIF mediated disease or disease state” refers to any and all disease states in which MIF plays a role, either by production or biological or enzymatic (tautomerase and/or oxidoreductase) activity of MIF itself, or by MIF causing or modulating another cytokine to be released, such as but not limited to IL-1, IL-2, IL-6, IL-8, IFN-γ and TNF. A disease state in which, for instance, IL-1 is a major component, and whose production or action, is exacerbated or secreted in response to MIF, would therefore be considered a disease state mediated by MIF.

As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to Macrophage Migration Inhibitory Factor (MIF), Interleukin-1 (IL-1), Interleukin-2 (IL-2), Interleukin-6 (IL-6), Interleukin-8 (IL-8), Tumor Necrosis Factor-alpha (TNF-α) and Tumor Necrosis Factor-beta (TNF-β).

As used herein, the term “cytokine interfering” or “cytokine suppressive amount” refers to an effective amount of a compound of Formula I or II which will cause a decrease either in the biological activity or the level of the cytokine present in vivo or in vitro, or the in vivo level of the cytokine to normal or sub-normal levels, when given to a patient for the treatment of a disease state which is exacerbated by, or caused by, excessive or unregulated cytokine production.

As used herein, the cytokine referred to in the phrase “inhibition of a cytokine for use in the treatment of a HIV-infected human” is a cytokine which is implicated in (a) the initiation and/or maintenance of T cell activation and/or activated T cell-mediated HIV gene expression and/or replication and/or (b) any cytokine-mediated disease associated problem such as cachexia or muscle degeneration.

As TNF-β (also known as lymphotoxin) has close structural homology with TNF-α (also known as cachectin) and since each induces similar biologic responses and binds to the same cellular receptor, both TNF-α and TNF-β are inhibited by the compounds of the present invention and thus are herein referred to collectively as “TNF” unless specifically delineated otherwise.

These inhibitor compounds of Formula I or II are of aid in determining the signaling pathways involvement in inflammatory responses. In particular, a definitive signal transduction pathway can be prescribed to the action of lipopolysaccharide in cytokine production in macrophages. In addition to those diseases already noted herein, treatment of stroke, neurotrauma/CNS head injury, cardiac, brain and renal reperfusion injury, thrombosis, glomerulonephritis, diabetes and pancreatic cells, multiple sclerosis, muscle degeneration, eczema, psoriasis, sunburn, and conjunctivitis are also included.

It is also recognized that both IL-6 and IL-8 are produced during rhinovirus (HRV) infections and contribute to the pathogenesis of common cold and exacerbation of asthma associated with HRV infection (Turner et al., (1998), Clin. Infec. Dis., Vol. 26, p. 840; Teren et al. (1997), Am. J. Respir. Crit. Care Med., Vol. 155, p. 1362; Grunberg et al. (1997), Am. J. Respir. Crit. Care Med., Vol. 156, p. 609 and Zhu et al., J. Clin. Invest. (1996), Vol. 97, p 421). It has also been demonstrated in vitro that infection of pulmonary epithelial cells with HRV results in production of IL-6 and IL-8 (Subauste et al., J. Clin. Invest. (1995), Vol. 96, p. 549). Epithelial cells represent the primary site of infection of HRV. Therefore, another aspect of the present invention is a method of treatment to reduce inflammation associated with a rhinovirus infection, not necessarily a direct effect of the virus itself.

Another aspect of the present invention involves the novel use of these cytokine inhibitors for the treatment of chronic inflammatory or proliferative or angiogenic diseases, which are caused by excessive, or inappropriate angiogenesis. Chronic diseases which have an inappropriate angiogenic component are various ocular neovascularizations, such as diabetic retinopathy and macular degeneration. Other chronic diseases which have an excessive or increased proliferation of vasculature are tumor growth and metastasis, atherosclerosis and certain arthritic conditions. Therefore, cytokine inhibitors will be of utility in the blocking of the angiogenic component of these disease states.

The term “excessive or increased proliferation of vasculature inappropriate angiogenesis” as used herein includes, but is not limited to, diseases which are characterized by hemangiomas and ocular diseases.

The term “inappropriate angiogenesis” as used herein includes, but is not limited to, diseases which are characterized by vesicle proliferation with accompanying tissue proliferation, such as occurs in cancer, metastasis, arthritis and atherosclerosis.

This invention also encompasses methods of treating or preventing disorders that can be treated or prevented by the inhibition of ERK/MAP in a mammal, preferably a human, comprising administering to said mammal an effective amount of a compound of Formula I or II. Accordingly, the present invention provides a method of treating an ERK/MAP kinase mediated disease in a mammal in need thereof, preferably a human, which comprises administering to said mammal, an effective amount of a compound of Formula I or II or a pharmaceutically acceptable salt thereof.

Preferred ERK/MAP mediated diseases for treatment include, but are not limited to psoriatic arthritis, Reiter's syndrome, rheumatoid arthritis, gout, traumatic arthritis, rubella arthritis and acute synovitis, rheumatoid spondylitis, osteoarthritis, gouty arthritis and other arthritic conditions, sepsis, septic shock, endotoxic shock, gram negative sepsis, toxic shock syndrome, Alzheimer's disease, stroke, ischemic and hemorrhagic stroke, neurotrauma/closed head injury, asthma, adult respiratory distress syndrome, chronic obstructive pulmonary disease, cerebral malaria, meningitis, chronic pulmonary inflammatory disease, silicosis, pulmonary sarcostosis, bone resorption disease, osteoporosis, restenosis, cardiac reperfusion injury, brain and renal reperfusion injury, chronic renal failure, thrombosis, glomerularonephritis, diabetes, diabetic retinopathy, macular degeneration, graft vs. host reaction, allograft rejection, inflammatory bowel disease, Crohn's disease, ulcerative colitis, neurodegenerative disease, multiple sclerosis, muscle degeneration, diabetic retinopathy, macular degeneration, tumor growth and metastasis, angiogenic disease, rhinovirus infection, peroral disease, such as gingivitis and periodontitis, eczema, contact dermatitis, psoriasis, sunburn, and conjunctivitis.

The term “treating”, as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

This invention also encompasses pharmaceutical compositions for the treatment of a condition selected from the group consisting of arthritis, psoriatic arthritis, Reiter's syndrome, gout, traumatic arthritis, rubella arthritis and acute synovitis, rheumatoid arthritis, rheumatoid spondylitis, osteoarthritis, gouty arthritis and other arthritic conditions, sepsis, septic shock, endotoxic shock, gram negative sepsis, toxic shock syndrome, Alzheimer's disease, stroke, neurotrauma, asthma, adult respiratory distress syndrome, cerebral malaria, chronic pulmonary inflammatory disease, silicosis, pulmonary sarcoidosis, bone resorption disease, osteoporosis, restenosis, cardiac and renal reperfusion injury, thrombosis, glomerularonephritis, diabetes, graft vs. host reaction, allograft rejection, inflammatory bowel disease, Crohn's disease, ulcerative colitis, multiple sclerosis, muscle degeneration, eczema, contact dermatitis, psoriasis, sunburn, or conjunctivitis shock in a mammal, including a human, comprising an amount of a compound of Formula I or II effective in such treatment and a pharmaceutically acceptable carrier.

One embodiment of the present invention provides a method for inactivating enzymatic and biological activity of human MIF comprising contacting the human MIF with a compound, or combination of compounds, that forms a stable interaction with at least one amino acid residue of the human MIF. The invention also relates to inhibiting other cytokines affected by MIF activity including IL-1, IL-2, IL-6, IL-8, IFN-γ and TNF. The invention encompasses methods of treating or preventing disorders that can be treated or prevented by the inhibition of the ERK/MAP pathway in a mammal, preferably a human, comprising administering to said mammal an effective amount of a compound.

As an example of the methods of treatment of the present invention, isoxazoline-containing compounds of the present invention can be used to treat patients with ARDS (acute respiratory distress syndrome). ARDS is often considered to be an archetypal clinical response in which the dynamic balance within the immune response shifts toward excessive inflammation and tissue destruction. MIF is expressed in both type II alveolar cells and infiltrating immune cells. MIF levels in the bronchoalveolar lavage of ARDS patients were found to be significantly elevated when compared to control subjects (Donnelly, et al., Nat. Med., 3, 320-323 (1997)). Human MIF enhances both TNFα and IL-8 secretion from ARDS alveolar macrophages (ex vivo) when compared to control cells. Pre-treatment of these cells with anti-MIF antibodies significantly decreases TNFα and IL-8 production from ARDS alveolar cells. Moreover, as discussed above under “Background of the Invention,” rMIF (recombinant MIF) was found to override, in a concentration-dependent fashion, glucocorticoid-mediated inhibition of cytokine secretion in ARDS macrophages. These were the first data to indicate that the MIF/glucocorticoid dyad is active in cells that had undergone pro-inflammatory activation in vivo during human disease (Donnelly, et al., Nat. Med., 3, 320-323 (1997). Significantly elevated levels of alveolar MIF were found in those at-risk patients who progressed to ARDS compared to those who did not. MIF likely acts as an important mediator to promote and sustain the pulmonary inflammatory response in ARDS. Its prominent expression in ARDS may explain the fulminant course of this disease and perhaps why glucocorticoid treatment has proven disappointing in established cases. Thus, pharmaceutical compositions comprising isoxazoline-containing compounds of the present invention can be used to treat ARDS patients.

As a further example of the methods of treatment of the present invention, isoxazoline-containing compounds of the present invention can be used to treat patients with rheumatoid arthritis. Synovial fluid obtained from the affected joints of patients with rheumatoid arthritis contain significantly greater levels of MIF than those obtained from patients with osteoarthritis or from normal control subjects (Metz, et al., Adv. Immunol., 66, 197-223 (1997); Leech, et al., Arthritis Rheum., 41, 910-917 (1998); Onodera, et al., Cytokine, 11, 163-167 (1999)). As revealed by immunohistochemical staining methods, infiltrating mononuclear cells within the human arthritic joint are the primary source of MIF. In two animal models of arthritis, neutralizing anti-MIF mAb's significantly inhibited disease progression and disease severity (Leech, et al., Arthritis Rheum., 41, 910-917 (1998); Mikulowska, et al., J. Immunol., 158, 5514-5517 (1997)) giving impetus to the desirability of developing additional MIF inhibitors for potential therapeutic use in inflammatory disease. Thus, pharmaceutical compositions comprising isoxazoline compounds or isoxazoline-related compounds of the present invention can be used to treat arthritis patients.

In yet a further example of the methods of treatment of the present invention, isoxazoline-containing compounds of the present invention can be used to treat patients with atopic dermatitis. Atopic dermatitis is a chronic pruritic inflammatory skin disorder. Its pathogenesis, in part, is thought to be due to dysregulated cytokine production by peripheral mononuclear cells. In lesions from patients with atopic dermatitis, MIF protein is diffusely distributed throughout the entire epidermal layer with increased expression by keratinocytes (Shimizu, et al., FEBS Lett., 381, 199-202 (1996)). In normal human skin, MIF has primarily been localized to epidermal ketatinocytes. The serum MIF level of atopic dermatitis patients were 6-fold higher than in control subjects. Additionally, serum MIF levels in atopic dermatitis patients decreased as clinical features improved, suggesting that MIF plays a pivotal role in the inflammatory response in the skin during dermatitis. Thus, pharmaceutical compositions comprising isoxazoline-containing compounds of the present invention can be used to treat patients with atopic dermatitis.

In a similar manner, the present invention also provides a method for treating or preventing other inflammatory or autoimmune disorders including, but not limited to, proliferative vascular disease, cytokine-mediated toxicity, sepsis, septic shock, psoriasis, interleukin-2 toxicity, asthma, MIF-mediated conditions, insulin-dependent diabetes, multiple sclerosis, graft versus host disease, lupus syndromes, and other conditions characterized by local or systemic MIF release or synthesis or by other cytokines affected by MIF.

In yet another example of the methods of treatment of the present invention, compounds of the present invention can be used to treat patients with tumor growth. Neutralizing anti-MIF antibodies have been found to significantly reduce growth and vascularization (angiogenesis) of mouse 38C13 B cell lymphoma in vivo (Chesney, et al., Mol. Med., 5, 181-191 (1999)). MIF was expressed predominantly in tumor-associated neovasculature. Cultured microvascular endothelial cells, but not 38C13 B cells, were observed both to produce MIF and to require its activity for proliferation in vitro (Takahashi, et al., Mol. Med., 4, 707-714 (1998)). In addition, the administration of anti-MIF antibodies to mice was found to significantly inhibit the neovascularization response elicited by Matrigel implantation, a model of new blood vessel formation in vivo (Bozza, et al., J. Exp. Med., 189, 341-346 (1999)). These data indicate that MIF plays an important role in tumor angiogenesis, a new target for the development of anti-neoplastic agents that inhibit tumor neovascularization.

Thus, the present invention also provides a method for treating or preventing tumor growth or angiogenesis, comprising administering an effective amount of a compound, or combination of compounds, having an isoxazoline moiety and that forms a stable interaction with at least one amino acid residue of an MIF protein.

The present invention also provides a compound of Formula I or II, or a pharmaceutically acceptable salt thereof, as a pharmaceutical composition comprising either of the aforesaid, for use in a medicine or for the manufacture of a medicament for the treatment or prevention of inflammatory disorders including arthritis, proliferative vascular disease, ARDS, cytokine-mediated toxicity, sepsis, septic shock, psoriasis, interleukin-2 toxicity, asthma, MIF-mediated conditions, autoimmune disorders (including, but not limited to, rheumatoid arthritis, insulin-dependent diabetes, multiple sclerosis, graft versus host disease, lupus syndromes), tumor growth or angiogenesis, or any condition characterized by local or systemic MIF release or synthesis.

This invention also encompasses pharmaceutical compositions for the treatment of a condition which can be treated by the inhibition of the ERK/MAP kinase pathway in a mammal, including a human, comprising an amount of a compound of Formula I or II effective in such treatment and a pharmaceutically acceptable carrier.

This invention also encompasses prodrugs of compounds of the Formula I or II and pharmaceutical compositions containing these prodrugs. Compounds of Formula I or II having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of Formula I or II. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain. The invention also encompasses sustained release compositions.

One of ordinary skill in the art will appreciate that the compounds of the invention are useful in treating a diverse array of diseases. One of ordinary skill in the art will also appreciate that when using the compounds of the invention in the treatment of a specific disease that the compounds of the invention may be combined with various existing therapeutic agents used for that disease.

For the treatment of rheumatoid arthritis, the compounds of the invention may be combined with agents such as TNF inhibitors such as anti-TNF monoclonal antibodies and TNF receptor immunoglobulin molecules, COX-2 inhibitors, such as celecoxib, rofecoxib, valdecoxib and etoricoxib, low dose methotrexate, lefunomide, hydroxychloroquine, d-penicillamine, auranofin or parenteral or oral gold.

The compounds of the invention can also be used in combination with existing therapeutic agents for the treatment of osteoarthritis. Suitable agents to be used in combination include standard non-steroidal anti-inflammatory agents such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc.

The compounds of the present invention may also be used in combination with anticancer agents such as endostatin and angiostatin or cytotoxic drugs such as adriamycin, daunomycin, cis-platinum, etoposide, taxol, taxotere and alkaloids, such as vincristine, farnesyl transferase inhibitors, VegF inhibitors, and antimetabolites such as methotrexate.

The compounds of the invention may also be used in combination with antiviral agents such as Viracept, AZT, aciclovir and famciclovir, and antisepsis compounds such as Valant.

The compounds of the present invention may also be used in combination with cardiovascular agents such as calcium channel blockers, lipid lowering agents such as statins, fibrates, beta-blockers, Ace inhibitors, Angiotensin-2 receptor antagonists and platelet aggregation inhibitors.

The compounds of the present invention may also be used in combination with osteoporosis agents such as roloxifene, droloxifene, lasofoxifene or fosomax and immunosuppressant agents such as FK-506 and rapamycin.

The compounds of the present invention may also be used in combination with CNS agents such as antidepressants, such as sertraline, anti-Parkinsonian drugs such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase, and anti-Alzheimer's drugs such as donepezil, tacrine, COX-2 inhibitors, propentofylline or metrifonate.

This invention also encompasses pharmaceutical compositions for the treatment of a condition which can be treated by the inhibition of ERK/MAP kinase in a mammal, including a human, comprising an amount of a compound of Formula I or II effective in such treatment and a pharmaceutically acceptable carrier.

The present invention further provides a method for treating inflammatory disorders including, but not limited to, arthritis, proliferative vascular disease, ARDS (acute respiratory distress syndrome), cytokine-mediated toxicity, sepsis, septic shock, psoriasis, interleukin-2 toxicity, asthma, MIF-mediated conditions, autoimmune disorders (including, but not limited to, rheumatoid arthritis, insulin-dependent diabetes, multiple sclerosis, graft versus host disease, lupus syndromes), tumor growth or angiogenesis, or any condition characterized by local or systemic MIF release or synthesis, comprising administering an effective amount of a compound having an isoxazoline moiety, wherein the isoxazoline moiety forms a stable covalent interaction with at least one amino acid residue of an MIF protein. Preferably, the interaction occurs at or near the active site of the tautomease activity of the MIF protein. The present invention also provides a pharmaceutical composition comprising a compound having an isoxazoline or isoxazoline-related moiety and a pharmaceutically acceptable carrier, wherein the moiety forms a stable covalent interaction with at least one amino acid residue of a MIF protein.

The present invention relates to compounds, compositions, processes of making, and methods of use related to inhibiting Macrophage Migration Inhibitory Factor (MIF) activity. The compounds comprise a genus of low molecular weight compounds comprising optionally substituted isoxazoline ring systems that act as inhibitors of MIF, and also inhibiting other cytokines affected by MIF activity including IL-1, IL-2, IL-6, LL-8, IFN-γ and TNF. This invention also encompasses methods of treating or preventing disorders that can be treated or prevented by the inhibition of the ERK/MAP pathway in a mammal, preferably a human, comprising administering to said mammal an effective amount of a compound. The compounds are useful for treating a variety of diseases involving any disease state in a human, or other mammal, which is exacerbated by or caused by excessive or unregulated MIF, IL-1, IL-2, IL-6, IL-8, IFN-γ and TNF production by such mammal's cells, such as, but not limited to, monocytes and/or macrophages, or any disease state that can be modulated by inhibiting the ERK/MAP pathway.

One embodiment of the invention provides a new class of MIF and other cytokine inhibitors structurally related to isoxazoline which are suitable to neutralize both endogenous and exogenous MIF and other cytokines. The present invention therefore provides a genus of inhibitor compounds. Compounds in this genus are generally described by the general Formulas I and II herein. Unless otherwise indicated, structural Formulas I and II and described substituents are as indicated herein.

Given the teachings herein, the compounds can be synthesized by a variety of routes known to the organic chemist having ordinary skill in the art.

### EXAMPLES

Having generally described this invention, a further understanding can be obtained by reference to certain specific examples, which are provided herein for purposes of illustration only and are not intended to be limiting unless otherwise specified.

### Example 1

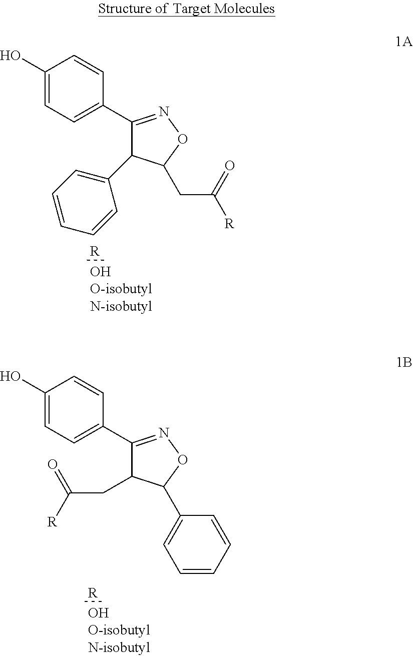

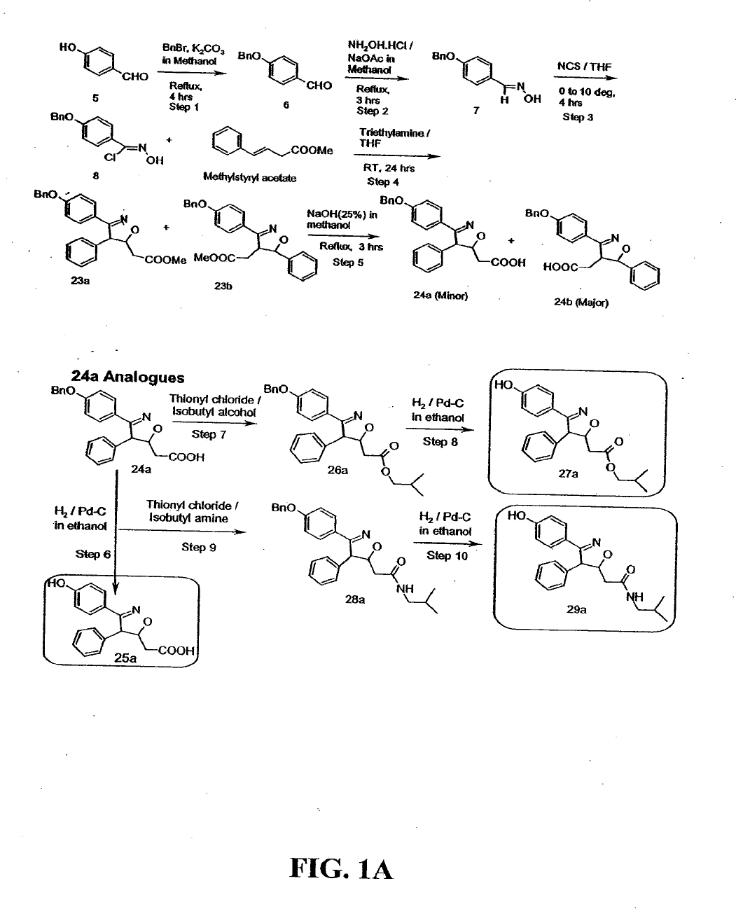

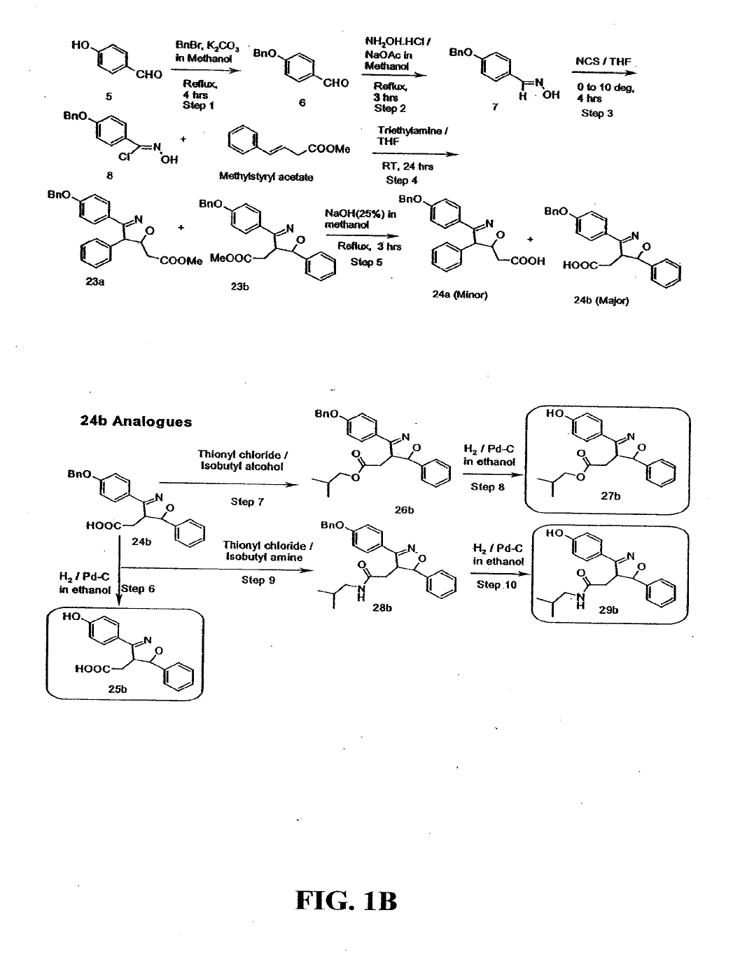

### Referring Now to the Phenyl Series Reaction Scheme in FIG. 1A:

To the solution of Chlorooxime (Compound 8, 14.8 g) in THF (100 ml) was added triethylamine (14.2 g) and the solution was cooled to 5-10 deg. To the above solution was added slowly methylstyryl acetate (5 g) and the resultant solution was stirred at RT for 24 hrs. The solvent was removed by distillation and the residue was dissolved in ethyl acetate (100 ml) and washed with water (2×50 ml) followed by brine solution. The organic layer was dried over anhydrous sodium sulfate, filtered, and concentrated to a residue. The TLC shows that two regioisomers were formed (Compounds 23a and 23b). The yellow solid which was a mixture of the two regioisomers (25 g) was taken on to the hydrolysis step.

The crude reaction mass (Compounds 23a and 23b, 25 g) was taken in methanol (200 ml) and 25% sodium hydroxide solution (13.0 ml) was added. The resultant solution was refluxed for 2 hrs. The solvent was removed by distillation and the residue was diluted with water (100 ml) and adjusted to a pH of 2 with hydrochloric acid (2M). The compound was extracted with ethyl acetate (2×200 ml). The organic layer was further washed with brine (100 ml). The resultant organic layer was dried over anhydrous sodium sulfate, filtered, and concentrated. The mixture of the two isomers (Compounds 24a and 24b) was purified by column chromatography (100-200 mesh silica gel, 50% Ethyl acetate-Pet.ether) to give 24a (0.700 g) as a white solid. The material was taken on without further characterization.

The benzylated acid derivative (Compound 24a, 0.300 g) was dissolved in ethanol (60 ml) and 10% palladium on carbon was added. The reaction mixture was hydrogenated using balloon pressure for four hours. The reaction mixture was filtered through a pad of celite and the bed was washed with ethanol (30 ml). The ethanol was evaporated to give a residue. The compound 25a was further purified by column chromatography using 60-120 mesh silica gel and 10% Methanol-Chloroform as eluent giving pure 25a (0.060 g) as an off-white solid. M.P: 203 —206° C.

### HPLC Conditions:

Column: Symmetry shield RP-18 (4.6×150) mm
Max:Mobile phase: 0.01M KH2PO4 (PH=2.5):Acetonitrile (70:30)
Flow rate: 1.0; Wavelength: 215 nm
Retention time: 13.33; Purity: 92.38%
IR (KBr, ν max): 3382, 3035, 1704, 1607, 1516, 1433, 1219, 1173, 872.

1H NMR: (DMSO-d6, 300 MHz); δ 12.3 (br.s, 1H), 9.9 (br.s, 1H), 7.3 (d, 2H), 7.2 (m, 5H), 6.8 (d, 2H), 4.8 (d, 1H), 4.7 (m, 1H), 2.7 (m, 2H).

Mass: m/z. 298 (M+1).

To the benzylated acid derivative (Compound 24a, 0.300 g) was added thionyl chloride (1 ml) at 0 deg and the resultant clear solution was stirred at RT for 30 min. The excess thionyl chloride was removed under reduced pressure (10 mm-Hg). To the residue was added isobutyl alcohol (1.6 g) and the reaction mixture was stirred at RT for 2 hrs. The solution was diluted with ethyl acetate (5 ml) and washed with water (2×5 ml). The organic layer was concentrated to a residue. The Compound 26a was purified by column chromatography (60-120 mesh silica gel, 20% Ethyl acetate-Pet.ether) to yield 26a (0.150 g) as a liquid.

The benzylated acid derivative (Compound 26a, 0.150 g) was dissolved in ethanol (15 ml) and 10% palladium on carbon was added. The reaction mixture was hydrogenated using balloon pressure for 4 hrs. The reaction mixture was filtered through a pad of celite and the bed was washed with hot ethanol (30 ml). The ethanol was evaporated to give a residue. The ester 27a was further purified by column chromatography using 100-200 mesh silica gel and 40% Ethyl acetate-Pet.ether as eluent to yield 27a (0.060 g) as a solid. M.P: 143-146 deg.

### HPLC Conditions:

### Column: Zorbax SB C-18 (4.6×250) mm

Max:Mobile phase: 0.1% TFA:Acetonitrile (50:50)
Flow rate: 1.0 mL/min; Wavelength: 275 nm
Retention time: 18.93 min; Purity: 95.10%
IR (KBr, ν max): 3407, 2961, 1707, 1608, 1516, 1428, 1346, 1279, 1213, 1169, 1050, 1005, 877, 700 cm-1. 1H NMR: (DMSO-d6, 300 MHz); δ 9.8 (s, 1H), 7.6 (d, 2H), 7.3-7.5 (m, 5H), 6.8 (d, 2H), 4.9 (d, 1H), 4.8 (m, 1H), 3.9 (d, 2H), 2.8 (2dd, 2H), 1.9 (m, 1H), 0.9 (d, 6H). Mass: m/z. 354 (M+1), 235;

To the benzylated acid derivative (Compound 24a, 0.300 g) was added thionyl chloride (1 ml) at 0 deg and the resultant clear solution was stirred at RT for 30 min. The excess thionyl chloride was removed under reduced pressure (10 mm-Hg) and to the residue was added isobutyl amine (1.46 g) and the reaction stirred at RT for 2 hrs. The solution was diluted with ethyl acetate (5 ml), washed with water (2×5 ml) and the organic layer was concentrated to a residue. The compound 28a was purified by column chromatography (60-120 mesh silica gel, 30% Ethyl acetate—Pet.ether) to give pure 28a (0.180 g) as a liquid. The compound was taken on to the next step without further characterization.

The benzylated amide derivative (Compound 28a, 0.150 g) was dissolved in ethanol (15 ml) and 10% palladium on carbon was added. The reaction mixture was hydrogenated using balloon pressure for four hours. The reaction mixture was filtered through a pad of celite and the bed was washed with hot ethanol (30 ml). The ethanol was evaporated to give a residue. The compound 29a was further purified by column chromatography using 100-200 mesh silica gel and 40% Ethyl acetate-Pet.ether as eluent to give the desired product 29a as a solid (0.060 g).

### M.P: 144-149° C.

### HPLC Conditions:

### Column: Symmetry C-18 (4.6×250) mm

Max:Mobile phase: 0.01 M KH2PO4 (PH=2.5):Acetonitrile (60:40)
Flow rate: 0.6 mL/min; Wavelength: 270 nm
Retention time: 21.38 min; Purity: 92.35%
IR (KBr, ν max): 3373, 2959, 1647, 1607, 1517, 1440, 1273, 1171, 882, 839, 700 cm-1. 1M NMR: (CDCl3, 300 MHz), δ 7.5 (d, 2H), 7.3 (m, 5H), 6.8 (d, 2H), 6.1-6.2 (2br.s, 2H), 4.8 (m, 1H), 4.7 (d, 1H), 3.1 (m, 2H), 2.7 (m, 2H), 1.8 (m, 1H), 0.8 (m, 6H). Mass: nth 353 (M+1), 335, 232.

Referring Now to the Phenyl Series Reaction Scheme in FIG. 1B:

To the solution of Chlorooxime (Compound 8, 14.8 g) in THF (100 ml) was added triethylamine (14.2 g) and the solution was cooled to 5-10 deg. To the above solution was added slowly Methylstyryl acetate (5.0 g) and the resultant solution was stirred at RT for 24 hrs. The solvent was then removed by distillation and the residue was dissolved in ethyl acetate (100 ml) and washed with water (2×50 ml) followed by brine solution. The organic layer was dried over anhydrous sodium sulfate and concentrated to a residue. The TLC shows that two regioisomers were formed (Compounds 23a and 23b). The yellowish solid mixture of the two regioisomers (crude mass 25 g) was taken into the hydrolysis step.

The crude 23a and 23b (25.0 g) were taken in methanol (200 ml) and sodium hydroxide solution (25%, 3.24 g) was added and the resultant solution was refluxed for 2 hrs. The solvent was removed by distillation and the residue was diluted with water (100 ml) and acidified to a PH of 2 with hydrochloric acid (2M). The compound was extracted with ethyl acetate (2×200 ml). The organic layer was further washed with brine (100 ml). The resultant organic layer was dried over anhydrous sodium sulfate, filtered and concentrated in vacuo. The mixture of two isomers (Compounds 24a and 24b) was further purified by column chromatography (100-200 mesh silica gel, 50% Ethyl acetate-Pet.ether) to yield 24b (1.1 g) as a white crystalline solid. The compound was taken on to the next step.

The benzylated acid derivative (24b, 0.300 g) was dissolved in ethanol (60 ml) following which 10% Palladium on carbon (0.060 g) was added. The reaction mixture was hydrogenated using balloon pressure for four hours. The reaction mixture was filtered over through a bed of Celite and the bed was washed with ethanol (30 ml). The ethanol was evaporated to give 25b as a residue. The compound 25b was further purified by column chromatography using 60-120 mesh silica gel and 10% Methanol-Chloroform as eluent to yield 25b as on off white solid (0.050 g) (m.p. 153-159° C.).

IR (KBr, ν max): 3150, 1707, 1600, 1517, 1437, 1350, 1275, 961, 755 cm-1. 1H NMR: (CD₃OD, 300 MHz); δ 7.3 (d, 2H), 7.2 (m, 5H), 6.8 (d, 2H), 5.5 (d, 1H), 4.0 (m, 1H), 2.7 (m, 2H). Mass: m/z. 296 (M−1), 252, 171, 133.

To the benzylated acid derivative (24b, 0.300 g) was added thionyl chloride (1 ml) at 0 deg. The resultant clear solution was stirred at RT for 30 min. The excess of thionyl chloride was removed under reduced pressure (10 mm-Hg). To the residue was added isobutyl alcohol (1.6 g) and the solution stirred at RT for 2 hrs. The solution was diluted with ethyl acetate (5 ml) and extracted with water (2×5 ml). The organic layer was concentrated to a residue. The Compound 26b was purified by column chromatography (60-120 mesh silica gel, 20% Ethyl acetate-Pet.ether). The product was isolate (180 mg) as a liquid.

The benzylated acid derivative (Compound 26b, 0.150 g) was dissolved in ethanol (15 ml) and Palladium on carbon (0.030 g) was added. The reaction mixture was hydrogenated using balloon pressure for 4 hrs. The reaction mixture was filtered through a pad of celite and the bed was washed with hot ethanol (30 ml). The ethanol was evaporated to give a residue. The compound 27b was further purified by column chromatography using 100-200 mesh silica gel and 40% Ethyl acetate-Pet.ether as eluent to give the desired 27b as an off-white solid (80 mg).

### M.P.: 136-143° C.

### HPLC Conditions:

Column: Symmetry shield RP-18 (4.6×150) mm
Mobile phase: 0.01M KH2PO4 (PH=2.5):Acetonitrile (40:60)
Flow rate: 1.0 mL/min.; Wavelength: 275 nm
Retention time: 7.13 min.; Purity: 96.4%

IR (KBr, ν max): 3174, 2965, 1735, 1601, 1517, 1350, 1274, 1171, 752 cm-1.

1H NMR: (CD3OD, 300 MHz); δ 7.6 (d, 2H), 7.3-7.5 (m, 5H), 6.8 (d, 2H), 5.5 (d, 1H), 4.1 (m, 1H), 3.9 (m, 2H), 2.8 (2dd, 2H), 1.9 (m, 1H), 0.9 (d, 6H). Mass: m/z. 354 (M+1), 335, 307.

To the benzylated acid derivative (Compound 24b, 0.300 g) was added thionyl chloride (1 ml) at 0 deg and the resultant clear solution was stirred at RT for 30 min. The excess of thionyl chloride was removed under reduced pressure (10 mm-Hg). To the residue was added isobutyl amine (2 ml) and the solution was stirred at RT for 2 hrs. The solution was then diluted with ethyl acetate (5 ml) and washed with water (2×5 ml). The organic layer was concentrated in vacuo to a residue. The compound 28b was purified by column chromatography (60-120 mesh silica gel, 30% Ethyl acetate-Pet.ether) to give 28b (170 mg) as a liquid

To a solution of benzylated amide derivative (Compound 28b, 0.150 g)) in ethanol (15 ml) was added Palladium on carbon and the reaction mixture was hydrogenated using balloon pressure at rt for four hours. The reaction mixture was filtered through a bed of celite and the bed was washed with hot ethanol (30 ml). The ethanol was evaporated to give a residue. The compound 29b was further purified by column chromatography using 100-200 mesh silica gel and 40% Ethyl acetate-Pet.ether as eluent to give 0.060 g of pure 29b as a solid. M.P: 185-190° C.

### HPLC Conditions:

### Column: Symmetry C-18 (4.6×250) mm

Mobile phase: 0.05% TFA:Acetonitrile (55:45)
Flow rate: 1.0 mL/min.; Wavelength: 210 nm
Retention time: 13.57 min.; Purity: 98.16%
IR (KBr, ν max): 3396,
2961, 1649, 1606, 1544, 1441, 1346, 1278, 1241, 1172, 839, 747 cm-1.

1H NMR: (DMSO-d6, 300 MHz), δ 8.1 (t, 1H), (br.s, 1H), 7.5 (d, 2H), 7.3 (m, 5H), 6.8 (d, 2H), 5.5 (d, 1H), 4.0 (m, 1H), 3.0 (m, 2H), 2.5 (m, 2H), 1.7 (m, 1H), 0.8 (m, 6H). Mass: m/z 353 (M+1), 335, 234.

### Example 2

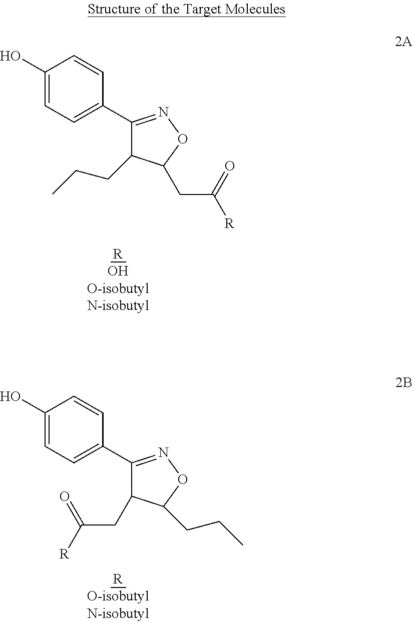

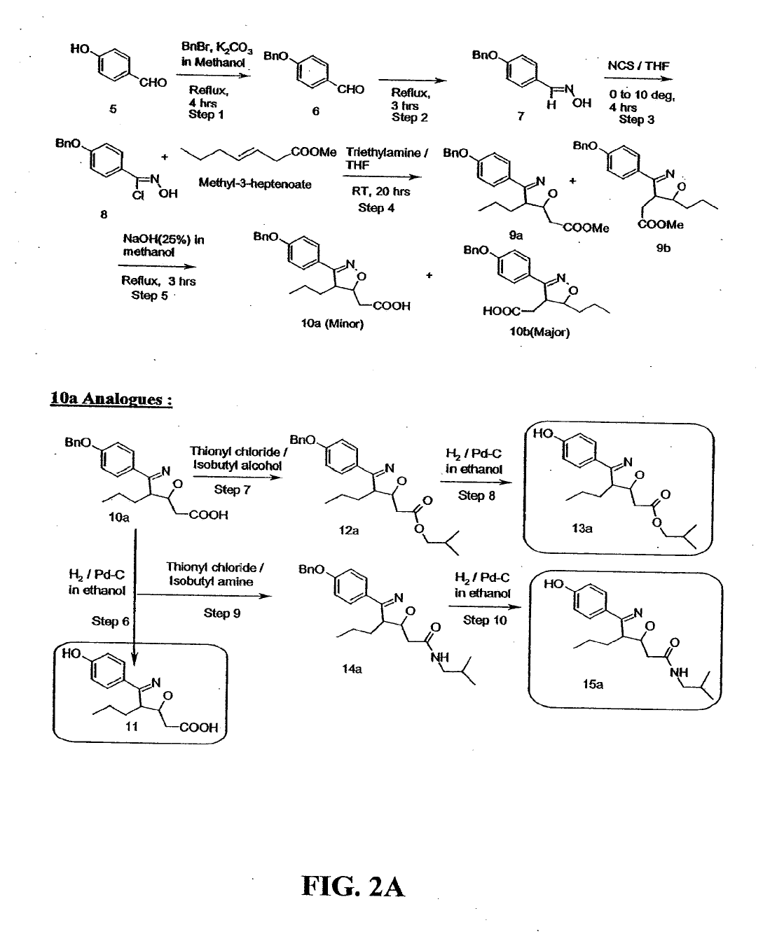

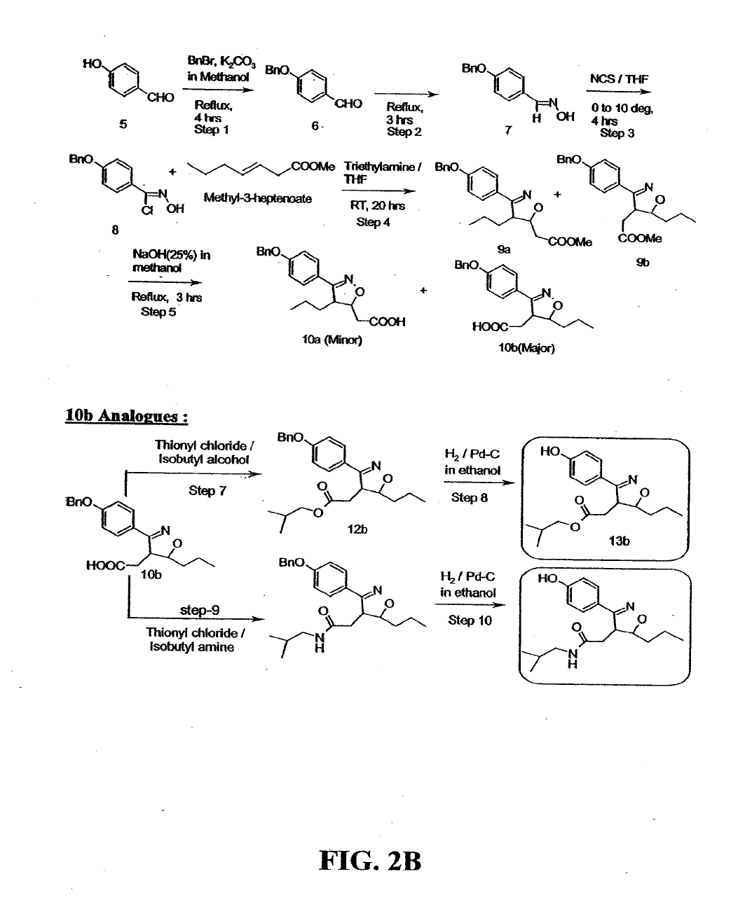

Referring Now to the Propyl Series Reaction Scheme in FIG. 2A:

To the solution of 4-Hydroxybenzaldehyde (Compound 5, 10 g) in THF (200 ml), was added potassium carbonate (16.95 g) followed by benzyl bromide (16.8 g) and the resultant reaction mixture was refluxed for 24 hrs. The reaction mixture was cooled to RT and the THF was removed under reduced pressure (10 mm-Hg). The residue was dissolved in ethyl acetate (100 ml) and washed with water (100 ml) followed by brine (100 ml). The organic layer was dried over anhydrous sodium sulfate, filtered, and concentrated. Evaporation of the solvent gave residue which was triturated with pet.ether to give a crystalline solid. The solid compound was filtered, washed with pet.ether, and dried under reduced pressure (10 mm-Hg) to give an off-white crystalline solid (Compound 6, 15.6 g).

To the solution of benzylated derivative (Compound 6, 10 g) in methanol (100 ml) was added hydroxylamine hydrochloride (4.9 g) and sodium acetate (9.6 g). The resultant reaction mixture was refluxed for 3 hrs. The reaction mass was cooled to RT. The solvent was removed under reduced pressure (10 mm-Hg), the residue was dissolved in ethyl acetate (100 ml), and washed with water (100 ml) followed by brine (100 ml). The organic layer was dried over anhydrous sodium sulfate, filtered, and concentrated. Evaporation of the solvent gave a white crystalline solid which was rinsed with pet.ether and dried under reduced pressure (10 mm-Hg) to give compound 7 (8.0 g).

To the solution of Oxime derivative (Compound 7, 10 g) in THF (100 ml) was added N-chlorosuccinimide (8.8 g) in THF at 0 deg over a period of 30 minutes and the resultant solution was stirred at 0 to 5 deg for 2-3 hrs. The solvent was evaporated at 40 deg under reduced pressure. The residue was dissolved in ethyl acetate (100 ml) and washed with water (100 ml) followed by brine (100 ml). The organic layer was dried over anhydrous sodium sulfate, filtered, and concentrated to a residue. The residue was washed with hexane and dried under reduced pressure (10 mm-Hg) to give a light yellow solid (Compound 8, 11.0 g).

To the solution of Chlorooxime (Compound 8, 16.74 g) in THF (100 ml) was added triethylamine (14.2 g) and the reaction mixture was cooled to 5-10 deg. To this solution was added slowly methyl-3-heptenoate (4.5 g) and the resultant solution was stirred at RT for 24 hrs. The solvent was removed under reduced pressure (10 mm-Hg) and the residue was dissolved in ethyl acetate (100 ml), washed with water (2×50 ml) followed by a brine solution. The organic layer was dried over anhydrous sodium sulfate, filtered, and concentrated to a residue. The TLC shows that two regioisomers were formed (Structure 9a and Structure 9b). The crude mass of the two regioisomers (25 g) was taken on the for hydrolysis step.

The crude reaction mass (25 g) was taken in methanol (200 ml) and added sodium hydroxide solution (25%, 13.6 ml). The resultant solution was refluxed for 2 hrs. The solvent was removed by distillation and the residue was diluted with water (100 ml) and the pH was adjusted to 2 with hydrochloric acid (2M). The compound was extracted with ethyl acetate (2×200 ml). The combined organic layers was again washed with brine (100 ml) and the resultant organic layer was dried over anhydrous sodium sulfate, filtered, and concentrated. The mixture of two isomers was further purified by column chromatography (100-200 mesh silica gel, 50%. Ethyl acetate-Pet.ether) to give compound 10a (0.700 g) as a white solid.

The Acid derivative (Compound 10a, 0.300 g) was dissolved in ethanol (30 ml) and palladium on carbon (0.030 g) was added. The solution was hydrogenated using balloon pressure for 4 hours. The reaction mixture was filtered over a pad of celite and the bed was washed with ethanol (30 ml). The ethanol was evaporated to give a residue. The product was further purified by column chromatography using 60-120 mesh silica gel and 30% Ethyl acetate-Pet.ether as eluent to give 11a (0.060 g) as an off-white solid. M.P: 175-177 C

### HPLC Conditions:

Column: Symmetry shield RP-18 (4.6×150) nm
Max:Mobile phase: 0.01 M KH2PO4 (PH=2.5):Acetonitrile (65:35)
Flow rate: 1.0 ml/min; Wavelength: 210 nm
Retention time: 5.49 min.; Purity: 94.44%
IR (KBr, ν max): 3372, 3284, 2931, 1703, 1607, 1516, 1435, 1351, 1283, 1220, 1171, 943, 878, 834, 674 cm-1. 1H NMR: (DMSO-d6, 300 MHz); δ 12.2 (br.s, 1H), 10 (br.s, 1H), 7.5 (d, 2H), 6.8 (d, 2H), 4.7 (m, 1H), 3.5 (m, 1H), 2.5 (m, 2H), 1.2-1.4 (m, 4H), 0.8 (t, 3H). Mass: m/z, 263 (M+1), 219, 178.

To the Compound 10a (0.300 g) at 0 deg was added thionyl chloride (1 ml) and the resultant clear solution stirred at RT for 30 min. The excess thionyl chloride was removed under reduced pressure (10 mm-Hg) and to the residue was added isobutyl alcohol (1.6 g) and the resultant solution was stirred at RT for 2 hrs. The solution was diluted with ethyl acetate (5 ml) and washed with water (2×5 ml). The organic layer was concentrated to a residue which was purified by column chromatography (60-120 mesh silica gel, 20% Ethyl acetate-Pet.ether) to give 12a (0.150 g) as a liquid.

The compound 12a (0.150 g) was dissolved in ethanol (15 ml) and 10% palladium on carbon (0.30 g) was added. The solution was hydrogenated using balloon pressure for 4 hrs. The reaction mixture was filtered over a pad of celite and the bed was washed with hot ethanol (30 ml). The ethanol was evaporated to give a residue which was further purified by column chromatography using 100-200 mesh silica gel and 30% Ethyl acetate-Pet.ether as eluent to give 13a (0.060 g) as a liquid. Yield: 60 mg

### HPLC Conditions:

### Column: Symmetry Sheild RP-18 (4.6×150)

Max:Mobile phase: 0.01 M KH2PO4 (PH=5):Acetonitrile
Flow rate: 1.0 ml/min; Wavelength: 270 nm
Retention time: 5.82 min; Purity: 96.30%
IR (KBr, ν max): 3390, 2961, 1729, 1607, 1516, 1464, 1350, 1272, 1173, 738 cm-1. 1H NMR: (CDCl3, 300 MHz); δ 7.5 (d, 2H), 6.9 (d, 2H), 4.8 (m, 1H), 3.9 (d, 2H), 3.4 (m, 1H), 2.6 (2dd, 2H), 1.9 (m, 1H), 1.3-1.5 (m, 4H), 0.8 (m, 9H). Mass: m/z 320 (M+1).

To compound 10a (0.300 g) was added thionyl chloride (1 ml) at 0 deg and the resultant clear solution stirred at RT for 30 min. The excess of thionyl chloride was removed under reduced pressure (10 mm-Hg) and to the residue was added isobutyl amine (1.46 g) and the solutions was stirred at RT for 2 hrs. The solution was diluted with ethyl acetate (5 ml) and extracted with water (2×5 ml). The organic layer was concentrated to a residue which was purified by column chromatography (60-120 mesh silica gel, 30% Ethyl acetate-Pet.ether) to give 14a (0.180 g) as a liquid.

The compound 14a (0.150 g) was dissolved in ethanol (15 ml) then 10% palladium on carbon (0.030 g) was added. The solution was hydrogenated using balloon pressure for four hours. The reaction mixture was filtered through a pad of celite and the bed was washed with hot ethanol (30 ml). The ethanol was evaporated to give a residue which was further purified by column chromatography using 100-200 mesh silica gel and 30% Ethyl acetate-Pet.ether as eluent to give 15a (0.060 g) as a solid. M.P: 136.6-143.5 deg.

### HPLC Conditions:

### Column: Zorbax SB C-18 (4.6×250) mm

Max:Mobile phase: Water:Acetonitrile (60:40)
Flow rate: 1.0 ml/min; Wavelength: 220 nm
Retention time: 10.28 min; Purity: 95.10%
IR (KBR, ν max): 3296, 2934, 1646, 1608, 1517, 1462, 1352, 1278, 1172, 880, 838, 605 cm-1. 1H NMR: (CDCl3, 300MHz); δ 7.5 (d, 2H), 6.8 (d, 2H), 6.3 (br. t, 1H), 4.8 (m, 1H), 3.4 (m, 1H), 3.1 (m, 2H), 2.6 (2dd, 2H), 1.8 (m, 1H), 1.3-1.5 (m, 4H), 0.8 (m, 9H). Mass: m/z: 319 (M+1), 301, 200.

Referring now to the Propyl Series reaction scheme in FIG. 2B:

To the solution of 4-Hydroxybenzaldehyde (Compound 5, 10.0 g) in THF (200 ml), was added potassium carbonate (16.95 g) followed by benzyl bromide (16.8 g) and the resultant reaction mixture was refluxed for 24 hrs. The reaction mixture was cooled to RT and the THF was removed under reduced pressure (10 mm-Hg). The residue was dissolved in ethyl acetate (100 ml) and washed with water (100 ml) followed by brine (100 ml). The organic layer was dried over anhydrous sodium sulfate. Evaporation of the solvent gave residue. The residue was decanted with pet.ether gave crystalline solid. The solid compound was filtered and washed with pet.ether and dried under reduced pressure (10 mm-Hg) to give an off-white crystalline solid (Compound 6, 15.6 g). The compound was taken on without further characterization.

To the solution of benzylated derivative (Compound 6, 10.0 g) in methanol (100 ml) was added hydroxylamine hydrochloride (4.9 g) and sodium acetate (9.6 g). The resultant reaction mixture was refluxed for 3 hrs. The reaction mass was cooled to RT, the solvent was removed under reduced pressure (10 mm-Hg), and the residue was dissolved in ethyl acetate (100 ml) and washed with water (100 ml) followed by brine (100 ml). The organic layer was dried over anhydrous sodium sulfate, filtered and concentrated to give a white crystalline solid (compound 7), which was rinsed with pet.ether and dried under reduced pressure (10 mm-Hg) to give 8.0 g of 7. The compound was taken on without further characterization.

To the solution of Oxime derivative (Compound 7, 10.0 g) in THF (90 ml) was added N-chlorosuccinimide (8.8 g) in THF (10 ml) at 0 deg over a period of 30 minutes and the resultant solution was stirred at 0 to 5 deg for 2-3 hrs. The solvent was evaporated at 40 deg under reduced pressure. The residue was dissolved in ethyl acetate (100 ml) and washed with water (100 ml) followed by brine (100 ml). The organic layer was dried over anhydrous sodium sulfate, filtered, and concentrated to a residue. The residue was washed with hexane to give a crystalline solid (Compound 8) which upon drying under reduced pressure (10 mm-Hg) gave 11.0 g of chloro-oxime 8 as a light yellow semi solid. The product was taken on without further characterization.

To the solution of Chlorooxime (Compound 8, 16.74 g) in THF (100 ml) was added triethylamine (14.2 g) and the reaction mixture was cooled to 5-10 deg. To this solution methyl-3-heptenoate (4.5 g) was slowly added and the resultant solution was stirred at RT for 24 hrs. The solvent was removed under reduced pressure (10 mm-Hg). The residue was dissolved in ethyl acetate (100 ml) and washed with water (2×50 ml) and brine solution. The organic layer was dried over anhydrous sodium sulfate, filtered, and concentrated to a residue. The TLC shows that two regioisomers were formed (Structure 9a and Structure 9b). The yellow solid crude mass (25 g) of the two regioisomers was taken on to the hydrolysis step.

TLC System: 20% Ethyl acetate-Pet.ether. Rf: 0.4

The crude reaction mixture of 9a and 9b (25 g) was taken up in methanol (200 ml) and sodium hydroxide solution (25%, 13.6 ml) was added. The resultant solution was refluxed for 2 hrs. The solvent was removed by distillation and the residue was diluted with water (100 ml) and the pH adjusted to 2 with hydrochloric acid (2M). The solution was extracted with ethyl acetate (2×200 ml) and the combined organic layers was again washed with brine (100 ml). The resultant organic layer was dried over anhydrous sodium sulfate, filtered, and concentrated. The mixture of the two isomers (Compounds 10a and 10b) was further purified by column chromatography (100-200 mesh silica gel, 50%. Ethyl acetate-Pet.ether) to give 10b (1.3 g) as a white crystalline solid which was taken on without further characterization.

To the Compound 10b (0.300 g) at 0 deg was added thionyl chloride (1 ml) and the resultant clear solution stirred at RT for 30 min. Excess thionyl chloride was removed under reduced pressure (10 mm-Hg) and isobutyl alcohol (1.6 g) was added to the residue and the solution was stirred at RT for 2 hrs. The solution was diluted with ethyl acetate (5 ml) and washed with water (2×5 ml). The organic layer was concentrated to a residue and compound 12b was purified by column chromatography (60-120 mesh silica gel, 20% Ethyl acetate-Pet.ether) to give 12b (0.150 g) as a liquid. The material was taken on to the next step.

The compound 12b (0.150 g) was dissolved in ethanol (15 ml) and then Palladium on carbon (0.030 g) was added. The solution was hydrogenated using balloon pressure for 4 hrs. The solution was filtered through a pad of celite and the bed was washed with hot ethanol (30 ml). The ethanol was evaporated to give a residue which was further purified by column chromatography using 100-200 mesh silica gel and 30% Ethyl acetate-Pet.ether as eluent to yield 13b (70 mg) as a pale yellow liquid.

### HPLC Conditions:

### Column: Symmetry Sheild RP-18 (4.6×150) mm

Max:Mobile phase: 0.01 M KH2PO4 (PH=2.5):Acetonitrile (45:55)
Flow rate: 1.0 ml/min; Wavelength: 270 nm
Retention time: 8.99 min; Purity: 92.24%
IR (KBr, ν max): 3781, 3377, 2962, 1728, 1602, 1267, 1170, 738 cm-1. 1H NMR: (DMSO-d6, 300 MHz); δ 7.5 (d, 2H), 6.8 (d, 2H), 4.3 (m, 1H), 3.9 (m, 2H), 3.8 (m, 1H), 2.6 (2dd, 2H), 1.9 (m, 1H), 1.3-1.5 (m, 4H), 0.8 (m, 9H). Mass: m/z 320 (M+1), 302, 248, 192.

To compound 10b (0.300 g) was added thionyl chloride (1 ml) at 0 deg and the resultant clear solution stirred at RT for 30 min. The excess thionyl chloride was removed under reduced pressure (10 mm-Hg). To the residue was added isobutyl amine (1.46 g) and the solution was stirred at RT for 2 hrs. The solution was then diluted with ethyl acetate (5 ml) and washed with water (2×5 ml). The organic layer was concentrated to a residue. The residue was purified by column chromatography (60-120 mesh silica gel, 30% Ethyl acetate-Pet.ether) to yield 14b (0.180 g) as a liquid.

Compound 14b (0.150 g) was dissolved in ethanol (15 ml) and then Paladium on carbon (0.030 g) was added. The solution was hydrogenated using balloon pressure for four hours. The reaction mixture was filtered through a pad of celite and the bed was washed with hot ethanol (30 ml). The ethanol was evaporated to give a residue which was further purified by column chromatography using 100-200 mesh silica gel and 30% Ethyl acetate-Pet.ether as eluent to give 15b (0.080 g) as a pale brown semi solid.

### HPLC Conditions:

### Column: Symmetry Sheild RP-18 (4.6×150) mm

Max:Mobile phase: 0.01M KH2PO4:Acetonitrile (55:45)
Flow rate: 1.0 ml/min; Wavelength: 210 nm
Retention time: 6.27 min; Purity: 93.87%
IR (KBr, ν max): 3416, 3300, 2924, 1653, 1610, 1550, 1515, 1348, 1275, 809 cm-1. 1H NMR: (CDCl3, 300 MHz); δ 8.2 (br.s, 1H), 7.5 (d, 2H), 6.8 (d, 2H), 6.2 (m, 1H), 4.4 (m, 1H), 3.8 (m, 1H), 3.1 (m, 211), 2.6 (2dd, 2H), 1.8 (m, 1H), 1.3-1.5 (m, 4H), 0.8 (m, 9H). Mass: m/z: 319 (M+1), 301, 200.

### Example 3

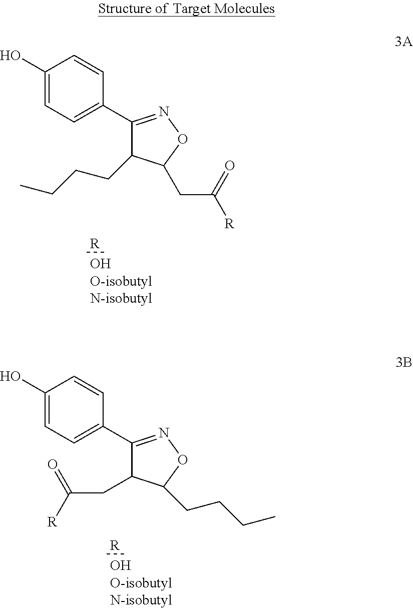

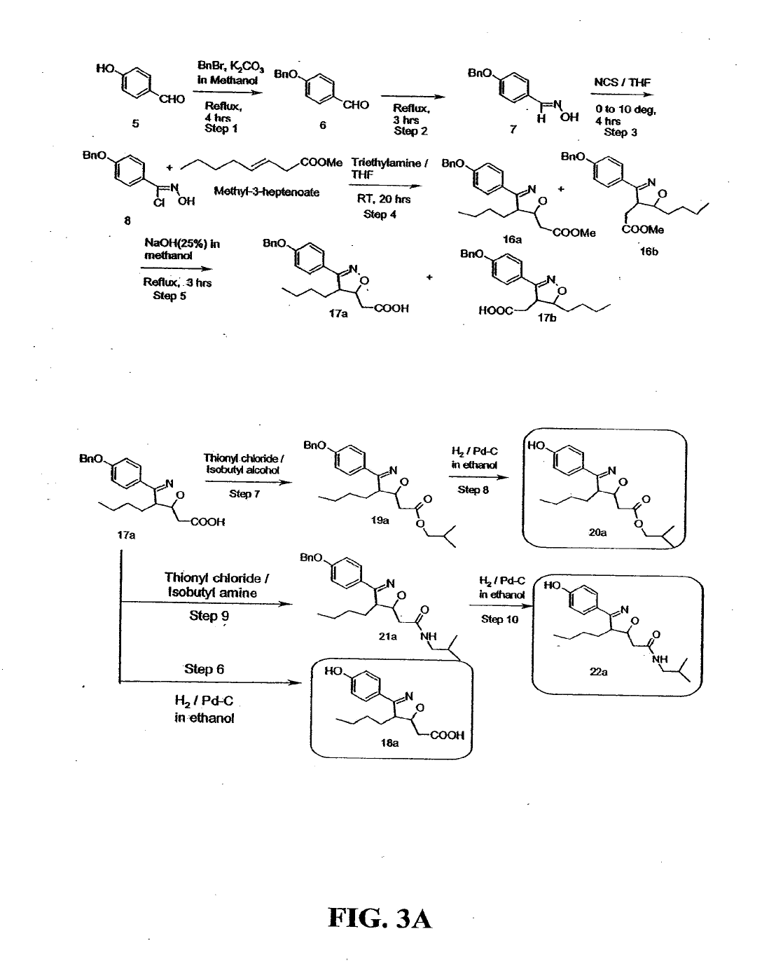

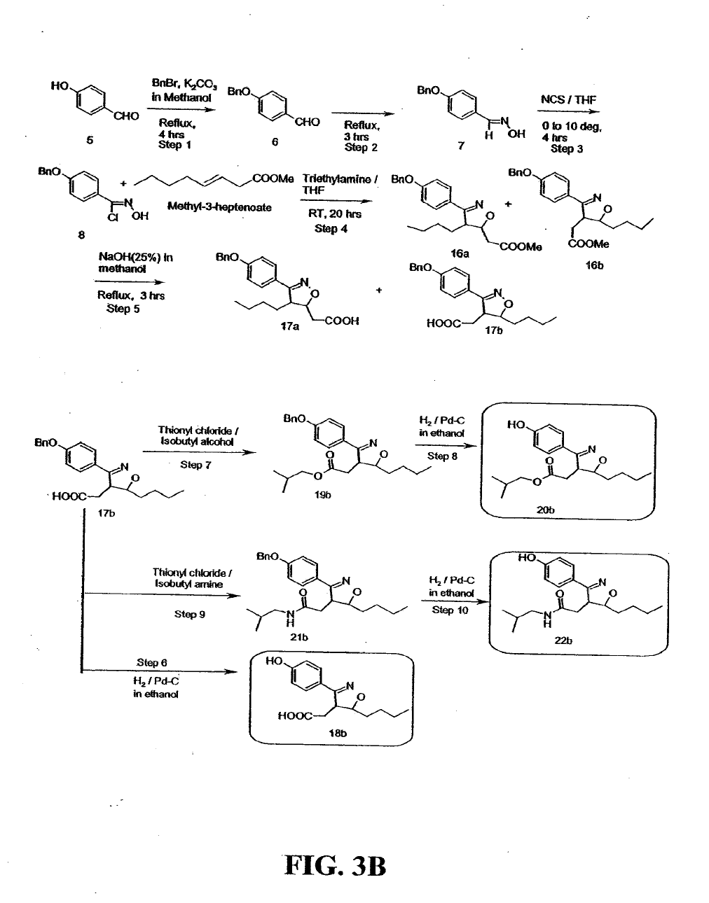

Referring now to the Butyl Series reaction scheme in FIG. 3A:

To the solution of Chlorooxime derivative (Compound 8, 16.74 g) in THF (100 ml) was added triethylamine (14.2 g) and the solution was cooled to 5-10 deg. To this solution was added slowly methyl-3-octenoate (5.0 g) and the resultant solution was stirred at RT for 24 hrs. The solvent was removed by distillation and the residue was dissolved in ethyl acetate (100 ml) and washed with water (2×50 ml) followed by brine. The organic layer was dried over anhydrous sodium sulfate, filtered, and concentrated to a residue. The TLC shows that two regioisomers were formed (Compounds 16a and 16b, 25 g) as a yellow solid. The crude material was taken on to the ester hydrolysis step.

The crude reaction mass (16a and 16b, 25 g) was taken in methanol (200 ml) and a 25% sodium hydroxide solution was added. The resultant solution was refluxed for 2 hrs. The solvent was removed under reduced pressure (10 mm-Hg) and the residue was diluted with water (100 ml) and adjusted to a pH of 2 with hydrochloric acid (2M). The solution was extracted with ethyl acetate (2×200 ml). The organic layer was again washed with brine (100 ml) and the resultant organic layer was dried over anhydrous sodium sulfate, filtered, and concentrated. The mixture was purified by column chromatography (100-200 mesh silica gel: 40% Ethyl acetate-Pet.ether) to give 17a (0.700 g) as a white solid.

The benzylated acid derivative (Compound 17a, 0.300 g) was dissolved in ethanol (60 ml) and palladium on carbon (0.060 g) was added. The solution was hydrogenated under balloon pressure for four hours. The reaction mixture was filtered through a pad of celite and the bed was washed with hot ethanol (30 ml). The ethanol was removed under reduced pressure to give a residue which was further purified by column chromatography using 60-120 mesh silica gel and 10% Methanol and Chloroform as eluent to give 18a (0.060 g) as an off-white solid.

### M.P: 174-176 C.°

### HPLC Conditions:

Column: Symmetry shield (4.6×150) mm

Mobile phase: 0.01M KH2PO4 (2.5):ACN (60:40)

Flow rate: 1.0 ml/min; Wavelength: 225 nm
Retention time: 5.53 min; Purity: 94.35%
IR (KBr ν max): 3406, 2930, 1701, 1606, 1516, 1434, 1351, 1283, 1221, 1170, 936, 828, 833, 675 cm-1. 1H NMR: (DMSO-d6, 300 MHz); δ 10 (s. br, 1H), 7.5 (d, 2H), 6.9 (d, 2H), 4.8 (m, 1H), 3.4 (m, 1H), 2.5 (m, 2H), 1.2-1.4 (m, 6H), 0.8 (t, 3H). Mass: m/z. 278 (M+1), 234, 192, 65.

To the benzylated acid derivative (Compound 17a, 0.300 g) was added thionyl chloride (1 ml) at 0 deg and the resultant clear solution stirred at RT for 30 min. The excess of thionyl chloride was removed under reduced pressure (10 mm-Hg) and to the residue isobutyl alcohol (1.6 g) was added and the solution stirred at RT for 2 hrs. The solution was diluted with ethyl acetate (5 ml) and washed with water (2×5 ml) followed by brine. The organic layer was concentrated to a residue which was further purified by column chromatography (60-120 mesh silica gel, Pet.ether and Ethyl acetate (10%)) to give 19a (0.150 g) as a liquid.

| | Chemicals, Reagents & | | | | |
|---|---|---|---|---|---|
| S. No | Solvents | M.Wt | mM | Eq | Qty |
| 1. | Benzylated ester derivative | 423.46 | 0.3542 | — | 150 mg |
| | (Compound 19) | | | | |
| 2. | Palladium on carbon | | | 20X | 30 mg |
| | (10% w/w) | | | | |
| 3 | Ethanol | | | 100X | 15 ml |

### Reaction Time: 4 hrs Reaction Temperature: 25 to 30 deg

The benzylated acid derivative (Compound 19a, 0.150 g) was dissolved in ethanol (15 ml) and 10% palladium on carbon was added. The solution was hydrogenated using balloon pressure for 4 hrs. The reaction mixture was filtered through a pad of celite and the bed was washed with hot ethanol (30 ml). The ethanol was evaporated to give a residue which was further purified by column chromatography using 100-200 mesh silica gel and 20% Ethyl acetate-Pet.ether as eluent to give 20a (0.060 g).

### HPLC Conditions:

Column: Symmetry shield (4.6×150) mm
Mobile phase: 0.01M KH2PO4 (PH=2.5):Acetonitrile (40:60)
Flow rate: 1.0 ml/min; Wavelength: 270 nm
Retention time: 7.51 min; Purity: 97.19%

IR (Kbr, ν max): 3378, 2960, 1729, 1606, 1517, 1464, 1352, 1276, 1173, 993, 888, 839 cm-1. 1H NMR: (CDCl₃, 300 MHz); δ 7.5 (d, 2H), 6.9 (d, 2H), 5.3 (br.s, 1H), 4.8 (m, 1H), 3.8 (d, 2H), 3.4 (m, 1H), 2.6 (2dd, 2H), 1.9 (m, 1H), 1.3-1.6 (m, 6H), 0.9 (d, 6H), 0.8 (t, 3H). Mass: m/z 334 (M+1), 192.

To the benzylated acid derivative (Compound 17a, 0.300 g) was added thionyl chloride (1 ml) at 0 deg and the resultant clear solution stirred at RT for 30 min. The excess of thionyl chloride was removed under reduced pressure (10 mm-Hg) and to the residue was added isobutyl amine (1.46 g) and the resultant solution stirred at RT for 2 hrs. The solution was diluted with ethyl acetate (5 ml) and washed with water (2×5 ml) followed by brine. The organic layer was concentrated to a residue which was further purified by column chromatography (60-120 mesh silica gel, 20% Ethyl acetate-Pet.ether) to give 21a (0.180 g) as a liquid.

The benzylated amide derivative (Compound 21a, 0.150 g) was dissolved in ethanol (15 ml) and 10% palladium on carbon was added. The solution was hydrogenated using balloon pressure for four hours. The reaction mixture was filtered through a pad of celite and the bed was washed with hot ethanol (30 ml). The ethanol was evaporated to give a residue which was further purified by column chromatography using 100-200 mesh silica gel and 20% Ethyl acetate-Pet.ether as eluent to give 22a (0.060 g) as an oily solid.

### HPLC Conditions:

### Column: Symmetry C-18 (4.6×250) mm

Max:Mobile phase: Water:Acetonitrile (40:60)
Flow rate: 0.8 ml min; Wavelength: 210 nm
Retention time: 5.65 min; Purity: 94.73%
IR (KBr ν max): 3298, 2959, 2930, 1646, 1607, 1517, 1461, 1353, 1278, 1172, 888, 838 cm-1. 1H NMR (CDCl3, 300 MHz): δ 7.5 (d, 2H), 6.8 (d, 2H), 6.1 (br.s, 1H), 4.8 (m, 1H), 3.4 (m, 1H), 3.2 (m, 2H), 2.6 (2dd, 2H), 1.8 (m, 1H), 1.3 (m, 4H), 0.8 (m, 9H). Mass m/z. 333 (M+1), 315, 308, 287, 286

Referring now to the Butyl Series reaction scheme in FIG. 3B:

The solution of Chlorooxime derivative (Compound 8, 16.74 g) in THF (100 ml) was added triethylamine (14.2 g) and the solution was cooled to 5-10 deg. To this solution was added slowly methyl-3-octenoate (5.0 g) and the resultant solution was stirred at RT for 24 hrs. The solvent was removed by distillation and the residue was dissolved in ethyl acetate (100 ml) and washed with water (2×50 ml) followed by brine. The organic layer was dried over anhydrous sodium sulfate, filtered, and concentrated to a residue. The TLC shows that two regioisomers were formed (Compounds 16a and 16b). The crude yellow solid (25 g) was taken into the hydrolysis step without further purification.

The mixture of 16a and 16b (25.0 g) was taken up in methanol (200 ml) and a 25% sodium hydroxide solution (13.6 ml) was added. The resultant solution was refluxed for 2 hrs. The solvent was removed under reduced pressure (10 mm-Hg) and the residue was diluted with water (100 ml) and adjusted to a pH of 2 with hydrochloric acid (2M). The solution was extracted with ethyl acetate (2×200 ml). The organic layer was again washed with brine (100 ml) and the resultant organic layer was dried over anhydrous sodium sulfate, filtered, and concentrated. The mixture of the two isomers (Compounds 17a and 17b) was further purified by column chromatography (100-200 mesh silica gel:40% Ethyl acetate-Pet.ether) to give a white solid (1.4 g) of compound 17b.

The benzylated acid derivative (Compound 17b, 0.300 g) was dissolved in ethanol (60 ml) and 10% palladium on carbon was added. The solution was hydrogenated under balloon pressure for four hours. The reaction mixture was filtered through a bed of celite and the bed was washed with hot ethanol (30 ml). The ethanol was removed under reduced pressure and the residue was further purified by column chromatography using 60-120 mesh silica gel and 10% Methanol and Chloroform as eluent. The compound 18b (0.080 g) was isolated as off-white crystals. mp: 163-168° C.

### HPLC Conditions:

Column: Symmetry shield C-18 (4.6×250) mm
Max:Mobile phase: 0.01M KH2PO4 (PH=2.5):Acetonitrile (50:50)
Flow rate 0.7 ml/min; Wavelength: 270 nm
Retention time: 6.87 min; Purity: 95.72%
IR (KBr ν max): 3209, 2958, 1711, 1612, 1597, 1519, 1434, 1352, 1274, 909_{;} 838 cm-1. 1H NMR: (DMSO-d6, 300 MHz); δ 10.0 (s. br, 1H), 7.5 (d, 2H), 6.9 (d, 2H), 4.5 (m, 1H), 3.7 (m, 1H), 2.5 (m, 2H), 1.5 (m, 2H), 1.2 (m, 4H), 0.8 (m, 3H). Mass: M/z 278 (M+1).

To the benzylated acid derivative (Compound 17b, 0.300 g) was added thionyl chloride (1 ml) at 0 deg and the resultant clear solution was stirred at RT for 30 min. The excess thionyl chloride was removed under reduced pressure (10 mm-Hg). To the residue was added isobutyl alcohol (1.6 g) and the solution stirred at RT for 2 hrs. The solution was diluted with ethyl acetate (5 ml) and washed with water (2×5 ml) followed by brine. The organic layer was concentrated to a residue. which was further purified by column chromatography (60-120 mesh silica gel, 20% Ethyl acetate-Pet.ether) to give 19b (0.150 g) as a liquid.

The benzylated acid derivative (Compound 19b, 0.150 g) was dissolved in ethanol (15 ml) then 10% palladium on carbon (0.030 g) was added. The solution was hydrogenated using balloon pressure for 4 hrs. The reaction mixture was filtered over a bed of celite and the bed was washed with hot ethanol (30 ml). The ethanol was evaporated to give a residue which was further purified by column chromatography using 100-200 mesh silica gel and 20% Ethyl acetate-Pet.ether as eluent. The desired ester 20b (0.060 g) was isolated as a solid. M.P: 114-116 deg.

### HPLC Conditions

Column: Symmetry shield RP-18 (4.6×150) mm
Max:Mobile phase: 0.01M KH2PO4 (PH=2.5):Acetonitrile (40:60)
Flow rate: 1.0 ml/min; Wavelength: 270 nm;
Retention time: 8.74 min; Purity: 97.76%
IR (KBr ν max): 3159, 2958, 2870, 1734, 1614, 1596, 1519, 1445, 1354, 1273, 1236, 1173, 898, 838 cm-1. 1H NMR: (CDCl3, 300 MHz); δ 7.5 (d, 2H), 6.9 (d, 2H), 5.8 (br. m, 1H), 4.4 (m, 1H), 3.9 (m, 2H), 3.7 (m, 1H), 2.6 (2dd, 2H), 1.9 (m, 1H), 1.3-1.6 (m, 6H), 0.8 (m, 9H). Mass: M/z. 334 (M+1).

To the benzylated acid derivative (Compound 17b, 0.300 g) was added thionyl chloride (1 ml) at 0 deg and the resultant clear solution stirred at RT for 30 min. The excess of thionyl chloride was removed under reduced pressure (10 mm-Hg). To the residue was added isobutyl amine (1.46 g) and the resultant solution stirred at RT for 2 hrs. The solution was diluted with ethyl acetate (5 ml) and washed with water (2×5 ml) followed by brine. The organic layer was concentrated to a residue. The Compound 21b was further purified by column chromatography (60-120 mesh silica gel, 20% Ethyl acetate-Pet.ether) to give pure 21b (0.180 g) as a liquid. The compound was taken on without further characterization.

The benzylated amide derivative (Compound 21b, 0.150 g) was dissolved in ethanol (15 ml) then 10% palladium on carbon was added. The solution was hydrogenated using balloon pressure for four hours. The reaction mixture was filtered through a pad of celite and the bed was washed with hot ethanol (30 ml) The ethanol was evaporated to give a residue. The compound 22b was further purified by column chromatography using 100-200 mesh silica gel and 20% Ethyl acetate—Pet.ether as eluent to yield pure 22b (0.060 g) as a solid. M.P: 157-161 deg.

### HPLC Conditions:

### Column: Symmetry C-18 (4.6×150) mm

Max:Mobile phase: 0.01M KH2PO4 (PH=2.5):Acetonitrile (40:60)
Flow rate 1.0 ml min; Wavelength: 270 nm
Retention time 10.07 min; Purity: 91.43%
IR (KBr ν max): 3286, 2961, 1653, 1610, 1558, 1516, 1461, 1348, 1277, 1229, 886, 840 cm-1. 1H NMR: (CDCl₃, 300 MHz); δ 7.5 (d, 2H), 6.8 (d, 2H), 6.1 (br.s, 1H), 4.8 (m, 1H), 3.3 (m, 1H), 2.6 (2dd, 2H), 1.8 (m, 1H), 1.3 (m, 4H), 0.8 (m, 9H). Mass: M/z. 333 (M+1), 315, 214.

### Example 4

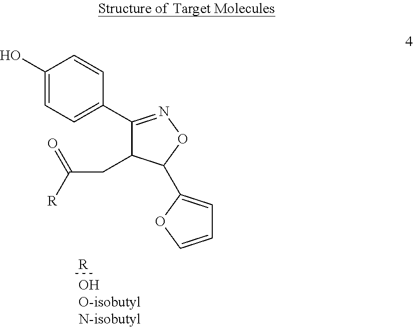

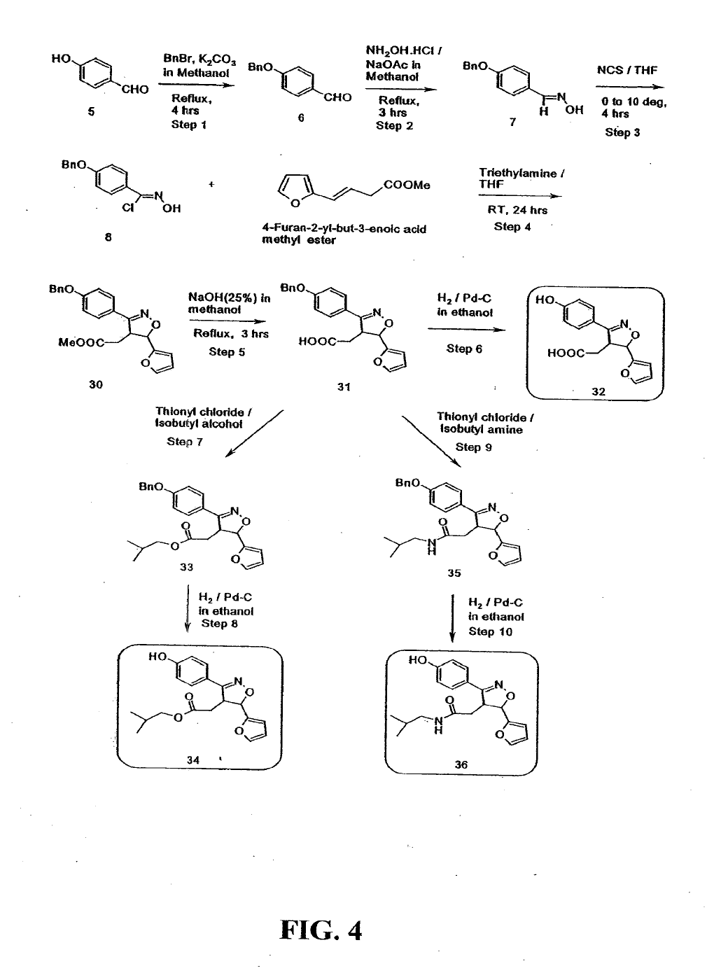

Referring now to the Furyl Series reaction scheme in FIG. 4:

To the solution of Chlorooxime (Compound 8, 15.7 g) in THF (100 ml) was added triethylamine 14.2 g) and the solution was cooled to 5-10 deg. To the above solution was added slowly 4-Furan-2-yl-but-3-enoic acid methyl ester (5.0 g) and the resultant solution was stirred at rt for 24 h. The solvent was removed by distillation and the residue was dissolved in ethyl acetate (100 ml) and washed with water (2×50 ml). The organic layer was dried over anhydrous sodium sulfate, filtered, and concentrated to a yellowish liquid (4.0 g). The TLC shows that only single isomer is formed. The crude reaction mass was taken on to the hydrolysis step.

The crude reaction mass (Compound 30, 1.5 g) was taken in methanol (20 ml) and sodium hydroxide solution (25%)(0.2 g) was added. The resultant solution was stirred at 25 to 30 deg for 16 hrs. The solvent was removed by distillation and the residue was diluted with water (100 ml) and adjusted the PH to 2 with hydrochloric acid (2M). The carboxylic acid derivative was extracted with ethyl acetate (2×200 ml) and the organic layer was further washed with brine (100 ml). The combined organic layers were dried over anhydrous sodium sulfate, dried, and concentrated. The compound 31 was further purified by column chromatography 100-200 mesh silica gel, 30% Ethyl acetate-Pet.ether as a solid (800 mg).

The benzylated acid derivative (Compound 31, 0.150 g) was dissolved in ethanol (15 ml) and Palladium on carbon (0.030 g) was added. The solution was hydrogenated using balloon pressure for four hours. The reaction mixture was filtered through a bed of celite and the bed was washed with hot ethanol (30 ml). The ethanol was evaporated to give a residue. The compound 32 was further purified by column chromatography using 100-200 mesh silica gel and 40% Ethyl acetate—Pet.ether as eluent to give 32 (0.060 g) as an off-white solid. M.P: 192-194° C.

### HPLC Conditions:

Column: Symmetry shield RP-18 (4.6×250) mm
Max:Mobile phase: 0.01M KH2PO₄ (PH=2.5):Acetonitrile (60:40)
Flow rate: 0.8 mL/min.; Wavelength: 210 nm
Retention time: 6.15 min.; Purity: 97.82%
IR (KBr. ν max): 3149, 2923, 1711, 1612, 1592, 1437, 1352, 1283, 911, 837, 745 cm-1. 1H NMR: (DMSO-d6, 300 MHz); δ 12.3 (br.s, 1H), 9.9 (br.s, 1H), 7.6 (s, 1H), 7.5 (d, 2H), 6.8 (d, 2H), 6.5 (d, 2H), 5.4 (d, 1H), 4.2 (m, 1H), 2.6 (m, 2H). Mass: m/z. 288 (M+1), 270, 242, 192, 164, 97.

To the benzylated acid derivative (Compound 31, 0.300 g) was added thionyl chloride (1 ml) at 0 deg and the resultant clear solution was stirred at RT for 30 min. The excess thionyl chloride was removed under reduced pressure (10 mm-Hg). To the residue was added isobutyl alcohol (1.6 g) and the solution was stirred at RT for 2 hrs. The solution was diluted with ethyl acetate (5 ml) and washed with water (2×5 ml). The organic layer was concentrated to a residue purified by column chromatography (60-120 mesh silica gel, 20% Ethyl acetate-Pet.ether). The product 33 was isolated as a liquid (0.180 g).

The benzylated acid derivative (Compound 33, 0.200 g) was dissolved in ethanol (15 ml) added Palladium carbon. The reaction mixture was hydrogenated using balloon pressure for four hours. The reaction mixture was filtered through a pad of celite and the bed was washed with hot ethanol (30 ml). The ethanol was evaporated to give a residue which was further purified by column chromatography using 100-200 mesh silica gel and 40% Ethyl acetate-Pet.ether as eluent to give 34 as an off-white solid (0.80 g). M.P: 123-125° C.

### HPLC Conditions:

### Column: Symmetry C-18 94.6×250) mm

Max:Mobile phase: 0.01M KH2PO4:Acetonitrile (50:50)
Flow rate: 1.0 mL/min.; Wavelength: 215 nm
Retention time: 13.58 min.; Purity: 95.97%
IR (KBr. ν max): 3781, 3221, 1735, 1598, 1517, 1440, 1348, 1276, 1228, 1175, 736 cm-1. 1H NMR: (CDCl₃, 300 MHZ), δ 7.5 (d, 2H), 7.3 (s, 1H), 6.9 (d, 2H), 6.5 (2d, 2H), 5.5 (d, 1H), 5.2 (br.s, 1H), 4.2 (m, 1H), 3.8 (d, 2H), 2.8 (2dd, 2H), 1.9 (m, 1H), 0.8 (d, 6H). Mass: ink 0.344 (M+1), 326, 248, 192, 151.

To the benzylated acid derivative (Compound 31, 0.500 g) at 0 deg was added thionyl chloride (1 ml) and the resultant clear solution stirred at RT for 30 min. The excess of thionyl chloride was removed under reduced pressure (10 mm-Hg). To the residue was added isobutyl amine (1.46 g) and the solution was stirred at RT for 2 hrs. The solution was then diluted with ethyl acetate (5 ml) and washed with water (2×5 ml). The organic layer was concentrated to a residue and the compound was purified by column chromatography (60-120 mesh silica gel, 40% Ethyl acetate-Pet.ether) to give 35 as a liquid (0.280 g) which was taken on without further characterization.

The benzylated acid derivative (35) was dissolved in ethanol (15 ml) and Palladium on carbon (10% w/w, 0.030 mg) was added. The solution was hydrogenated using balloon pressure for 4 hours. The reaction mixture was filtered through a bed of celite and the bed was washed with hot ethanol (30 ml). The ethanol was evaporated to give a residue which was further purified by column chromatography using 100-200 mesh silica gel and 40% Ethyl acetate-Pet.ether as eluent to give 36 (0.60 g) as an off-white solid. M.P: 167-169° C.

### HPLC Conditions:

### Column: Symmetry C-18 (4.6×150) mm

Max:Mobile phase: 0.01 M KH2PO4 (PH=2.5):Acetonitrile (60:40)
Flow rate: 1.0 mL/min.; Wavelength: 210 nm
Retention time: 8.05 min; Purity: 98.17%
IR (KBr. ν max): 3286, 2961, 1653, 1608, 1516, 1350, 1278, 1231, 1167, 841, 748. 1H NMR: (CDCl₃, 300 MHz), δ 7.5 (d, 2H), 7.3 (s, 1H), 6.9 (d, 2H), 6.3 (d, 2H), 5.4 (br.s, 1H), 5.3 (d, 1H), 4.3 (m, 1H), 3.1 (m, 2H), 2.6 (m, 2H), 1.8 (m, 1H), 0.8 (m, 6H). Mass: m/z 343 (M+1), 325, 275, 224.

The activity of the compounds of the invention for the various disorders described above can be determined according to one or more of the following assays.

### Materials and Methods

Synthesis.

In the examples of the syntheses that follow, all reagents and solvents used were purchased at the highest commercial quality. All solvents used were HPLC grade from Fisher. ¹H (270 MHz) and ¹³CNMR (67.5 MHz) NMR spectra were recorded on a JEOL Eclipse 270 spectrometer. Coupling constants were reported in Hertz (Hz), and chemical shifts were reported in parts per million (ppm) relative to tetramethylsilane (TMS, 0.0 ppm) with CDCl₃, DMSO or CD₃OD as solvent. Thin layer (TLC) and flash column chromatography were performed using Alumina B, F-254 TLC plates from Selecto Scientific and Fisher Scientific Basic alumina Brockman activity I, respectively. The reactions were monitored by TLC and ¹HNMR and were stopped when the yield of the crude according to ¹HNMR was 90-95%.

Reagents.

Unless otherwise indicated, all chemicals were purchased from Aldrich or Sigma Chemical Companies, and were of the highest grade commercially available. Dopachrome methyl ester was prepared similarly to previously published procedures (Bendrat, et al., Biochemistry, 36, 15356-15362 (1997); Swope, et al., EMBO J., 17, 3534-3541 (1998)).

Assays were initiated at a time when the absorbance at 475 nm reached a maximal value, signifying that the limiting reagent, NaIO₄, was consumed. Recombinant human and mouse MIF was expressed in E. coli and purified as previously reported (Bernhagan, et al., Biochemistry, 33, 14144-14155 (1994).

### MIF Tautomerase Activity

The compounds of Formula I or II are identified as MIF inhibitors because they inhibit MIF enzymatic activity in vitro. MIF catalyzes a tautomerization (i.e., keto-enol isomerization) reaction (Rosengren, et al., Molecular Medicine, 2, 143-149 (1996). MIF catalyzes the tautomerization of a dopachrome-related MIF substrate to a colorless product. Unless specifically indicated to the contrary, references made herein to an inhibitory concentration (e.g., IC₅₀ or other activity index) refer to the inhibitory activity of a test compound in an MIF tautomerase assay (as further described in detail below, and in Bendrat, et al., Biochemistry, 36, 15356-15362 (1997). The most active substrate identified is a non-physiological D-isomer of dopachrome. This reaction predicts therapeutic MIF inhibitors (see U.S. Pat. No. 6,420,188 and U.S. Pat. No. 6,599,938, the disclosures of which are incorporated herein by reference in their entirety). Inhibition of MIF tautomerase activity is predictive of inhibition of MIF biological activity.

A method for performing an assay for MIF dopachrome tautomerase activity begins with the preparation and oxidation of a DORA-related substrate precursor, such as L-3,4-dihydroxyphenylalanine methyl ester. This oxidation with sodium periodate generates the corresponding dopachrome derivative (e.g., L-3,5-dihydro-6-hydroxy-5-oxo-2H-indole-2-carboxylic acid methyl ester (“dopachrome methyl ester”) that is orange-colored and comprises a convenient substrate for use in a photometric assay for the enzymatic activity of MIF as a tautomerase. MIF (typically a purified preparation of recombinant MIF at a final concentration of 50-1000 ng/ml) addition causes the rapid tautomerization of the colored dopachrome substrate to a colorless 5,6-dihydroxyindole-2-carboxylic acid methyl ester product. The enzymatic activity of MIF is measured as the rate of de-colorization of the colored solution of the dopachrome-related substrate in a suitable buffer, typically at a time 20 seconds after addition of the final assay component and mixing. The absorbance is measured at about 475 nm (or 550 nm for substrate concentrations in excess 0.5 nM). A test compound may be included in the assay solution such that the effect of the test compound on MIF tautomerase activity (i.e., as an inhibitor) may be measured by noting the change in kinetics of substrate tautomerization compared to control assays performed in the absence of the test inhibitor compound. In particular, the MIF tautomerase assay may be conducted essentially as follows:

L-3,4-dihydroxyphenylalanine methyl ester (e.g., Sigma D-1507) is a dopachrome substrate precursor, and is prepared as a 4 mM solution in dd H₂O, Sodium periodate is prepared as an 8 mM solution in dd H₂O. Assay Buffer (50 mM potassium phosphate/1 mM EDTA, pH 6.0) is prepared. Purified recombinant MIF is prepared in 150 mM NaCl/20 mM Tris buffer (pH 7.4) as a stock solution convenient to supply MIF at a final concentration of about 700 ng/ml. Immediately prior to initiating the assay, 3.6 ml dopachrome substrate precursor solution, 2.4 ml periodate solution and 4.0 ml Assay Buffer are combined into a homogeneous mixture (this preparation of dopachrome substrate is suitable for assay use after 1 min and for about 30 min thereafter). Test compound (typically prepared as a concentrated stock in DMSO) and MIF are then combined with 0.7 ml Assay Buffer plus 0.3 ml dopachrome substrate solution to provide the desired final concentration of the test compound in a homogeneous mixture, and the optical density (absorbance) of this assay mixture is monitored at 475 nm. Typically, OD₄₇₅ is recorded every 5 sec for 0-60 sec, and the OD₄₇₅ for a given time point is compared to parallel assays where MIF is not added or the test compound is omitted. Inhibition of MIF tautomerase activity by the test compounds is determined by inhibition of the de-colorization of the assay mixture, often at the 20 sec time point. IC₅₀ values for compounds with MIF tautomerase inhibitory activity, corresponding to the concentration of inhibitor that would inhibit MIF tautomerase activity by 50%, are determined by interpolation of the results from MIF tautomerase assays at several different inhibitor concentrations. These IC₅₀ values provide a reasonable correlation between MIF enzymatic inhibitory activity of the test compounds, and inhibition of the biological activity of MIF.

The MIF tautomerase assay shows that certain compounds inhibit MIF enzymatic activity. The data provides a reasonable correlation between the MIF tautomerase enzymatic assay and MIF antagonism in a biological assay. Collectively, these data show that inhibition by a compound in the MIF tautomerase assay is predictive of its potential therapeutic use in inhibiting MIF biological activity. Inhibition of MIF is also reasonably correlated to the modulation of other cytokines affected by MIF and the ERK/MAPK pathway.

Treatment of MIF with Inhibitors.

MIF samples were treated with various concentrations of the inhibitors and treated MIF samples were then analyzed for enzyme activity using the dopachrome tautomerase assay.

### Dopachrome Tautomerase Assays.

To a room temperature solution of recombinant mouse or human MIF samples was added dopachrome methyl ester. The sample was immediately monitored for loss in absorbance at 475 nm compared to untreated MIF solutions and to dopachrome methyl ester without the addition of MIF.

### Enzyme Inhibition Studies.

This assay illustrates the inhibition of the enzymatic activity of human MIF by the compounds of the invention. The enzymatic tautomerization activity of recombinant human MIF was performed using L-dopachrome methyl ester as a chromogenic substrate (Bendrat, et al., Biochemistry, 36, 15356-15362 (1997)). The tautomerization reaction catalyzed by MIF, as described in detail above, leads to the formation of a dihydroxyindole product which is colorless.

Several compounds were prepared and tested for activity in the MIF dopachrome tautomerase assay. Compounds 68 and 69 (TABLE I) inhibited MIF tautomerase activity in a dose-dependent manner with an IC₅₀ of about 10 μM.

Thus, according to the present invention, the compounds related in structure to compound 68 and 69 comprise a new and general class of low molecular weight, specific inhibitors of MIF enzymatic activity.

### Biological Assay of MIF Activity.

This assay shows that the compounds not only specifically inhibit MIF enzymatic activity, but also inhibit MIF immunoregulatory activities, specifically, MIF glucocorticoid regulating activity. The ability of compounds according to the invention to neutralize the effect of MIF to influence the anti-inflammatory effect on TNFα production by human monocytes is tested. The property of the compound is dose dependent. To address the specificity of this inhibitory effect on MIF, other analogs are tested that are not such potent inhibitors of MIF tautomerase activity.

These results are consistent with a hypothesis that the pro-inflammatory effects of MIF can be neutralized by the binding of a small molecule at the tautomerase active site, although this effect is not believed to depend on the neutralization of tautomerase activity per se.

The compounds are additionally assessed for inhibition of MIF biological activities in any of a number of assays for MIF biological activity including, for example, inhibition of MIF binding to target cells, inhibition of MIF release or synthesis, inhibition of MIF immunoreactivity with MIF-specific antibodies, alterations of MIF conformation or structural integrity as assessed by circular dichroism spectroscopy, liquid NMR-spectroscopy, X-ray crystallography, thermal stability measurement, inhibition of the pro-proliferative effects of MIF on quiescent NIH/3T3 cells and inhibition of the associated prolonged ERK activation therein, inhibition of MIF-induced arachadonic acid release from NIH/3T3 cells, inhibition of MIF-induced fructose 2,6 bisphosphate formation in L6 myocytes, inhibition of MIF toxicity in the MIF, TNF, or LPS-challenged test animals, inhibition of the glucocorticoid counter-regulatory activity of MIF in vitro or in vivo, inhibition of the MIF-induced functional inactivation of the p53 tumor suppressor protein (Hudson, et al., J. Exp. Med., 190, 1375-1382 (1999), inhibition of MIF-induced release of prostaglandin E2, and inhibition of morbidity or mortality in any of a number of animal models of human diseases that are characterized by the release, production and/or appearance of MIF.

From the foregoing description, it can be seen that the present invention comprises a new and unique compounds, compositions, processes of making and methods of use related to the inhibition of MIF by the above compounds. It will be recognized by those skilled in the art that changes could be made to the above-described embodiments of the invention without departing from the broad inventive concepts thereof. It is understood, therefore, that this invention is not limited to the particular embodiments disclosed, but is intended to cover all modifications which are within the spirit and scope of the invention and that this invention is not limited to the particular embodiments disclosed, but it is intended to cover any modifications which are within the spirit and scope of the present invention.

1. A compound having Formula I or II: 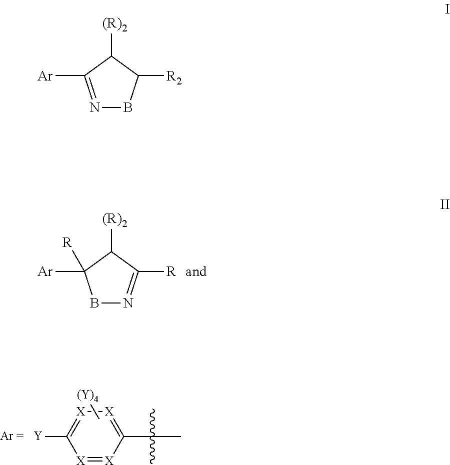
wherein B is oxygen or sulphur; and
each R is independently defined as follows: 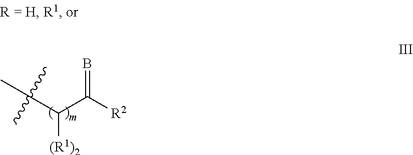
wherein in Formula I and Formula II, at least one R is not hydrogen;
wherein each R¹ is independently hydrogen, an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteraryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group;
each R² is independently an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteroaryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group
each m is independently zero or an integer from one to twenty; and
each X is independently carbon or nitrogen, wherein when any X is carbon, then each Y is defined independently as follows: 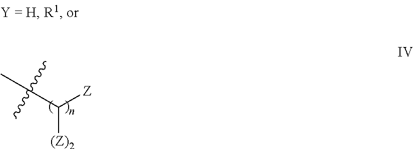
wherein each Z is independently hydrogen, an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteroaryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group; and
each n is independently zero or an integer from one to four;
pharmaceutically acceptable salts thereof and pharmaceutically acceptable prodrugs thereof.

2. The compound of claim 1, which is a compound having Formula I, a pharmaceutically acceptable salt thereof or a pharmaceutically acceptable prodrug thereof.

3. The compound of claim 1, which is a compound having Formula II, a pharmaceutically acceptable salt thereof or a pharmaceutically acceptable prodrug thereof.

4. The compound of claim 1, wherein at least one R in Formulas I and II has the following Formula III:

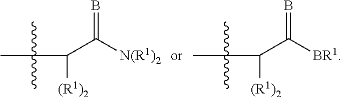

5. The compound of claim 1, wherein Ar in Formulas I and II is one of the following:

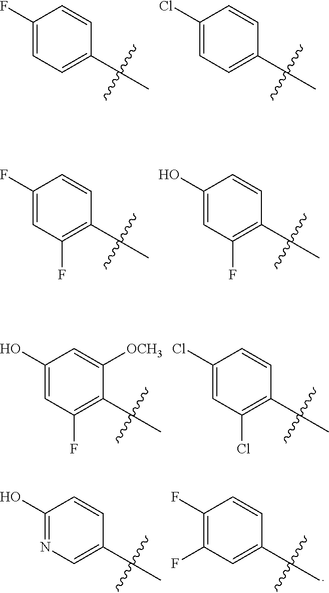

6. The compound of claim 1, wherein Ar is one of the following: 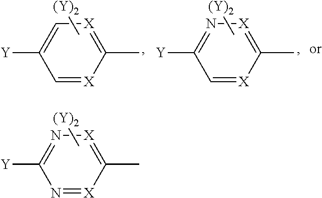
wherein X and Y are defined above.

7. The compound of claim 1, wherein B is oxygen.

8. The compound of claim 1, wherein R and R¹ are each independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—; wherein two independently chosen R¹ alkyl-containing groups may be taken together with any nitrogen atom to which they are attached to form a three to forty membered, cyclic heterocyclic or heteroaryl ring.

9. The compound of claim 1, wherein R and R¹ are each independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, and (C₁-C₆)alkoxy.

10. The compound of claim 1, wherein R and R¹ are each independently selected from the group consisting of hydrogen, (C₃-C₁₀)cycloalkyl, (C₁-C₁₀)alkoxy, (C₁-C₁₀)alkyl, phenyl, (C₁-C₁₀)heteroaryl, (C₁-C₁₀)heterocyclic and (C₃-C₁₀)cycloalkyl.

11. The compound of claim 1, wherein each R and R¹ are defined as independently selected from the group consisting of hydrogen, (C₃-C₆)cycloalkyl, (C₁-C₆)alkoxy, and (C₁-C₆)alkyl.

12. The compound of claim 1, having Formula IA: 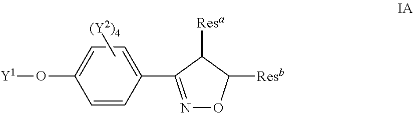
wherein
each Y¹ is independently hydrogen or (C₁-C₆)alkyl;
each Y² is independently Y¹, hydroxyl, halo, —N₃, —CN, —SH, or —N(Y¹)₂;
Res^{a} is independently Y¹, halo, —N₃, —CN, —OY¹, N(Y¹)₂, —SH, ═O, ═CH₂, or A, wherein each A is independently phenyl or an aromatic ring substituted with one or more independent Y² substituents;
Res^{b} is defined as follows: 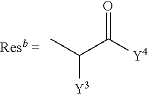
wherein Y³ is independently Y¹, A, —(CH₂)-A, —N(Y¹)₂, or —NY¹Y⁵, wherein each Y⁵ is independently a saturated or unsaturated, straight or branched (C₂-C₁₈)alkyl; and
wherein Y⁴ is independently a Y¹, —OY¹, —OY⁵, —N(Y¹)₂, —NY¹Y⁵, or A;
pharmaceutically acceptable salts thereof and pharmaceutically acceptable prodrugs thereof.

13. The compound of claim 1, having the following Formulas I or II 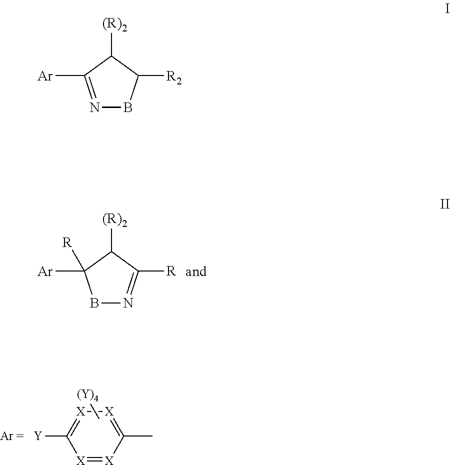
wherein B is oxygen or sulphur; and
each R is independently defined as follows: 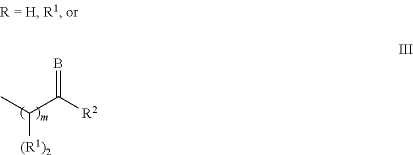
wherein in Formula I and Formula II, at least one R is not hydrogen;
each m is independently zero or an integer from one to twenty; and
each X is independently carbon or nitrogen, wherein when any X is carbon, then Y is defined independently for each carbon X as: 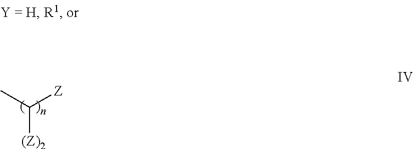
wherein each Z is independently hydrogen, hydroxyl, fluorine, bromine, iodine, —N₃, —CN, —SR³, —OR³, —N(R¹)₂, —R¹, or A; wherein each A is independently phenyl or an aromatic ring substituted with one or more independent Y² substituents; wherein each Y² is independently Y¹, hydroxyl, halo, —N₃, —CN, —SH, or —N(Y¹)₂; and wherein each Y¹ is independently hydrogen or (C₁-C₆)alkyl;
wherein n is independently zero or an integer from one to four;
wherein each R¹ is independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkyl-amino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—-[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heteroeyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂—N—(C═O)—, phenyl-NH—-(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents for R¹ may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkyl-amino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO(C═O)—, (C₁-C₆)alkyl-O—(C═O)—, H₂N(C═O)—(C₁-C₆)alkyl-NH—(C═O)—, [(C₁-C₆)alkyl]₂—N—(C═O)—, phenyl-NH—(C═O)—, phenyl-[((C₁-C₆)alkyl)-N]-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; and wherein two independently chosen R¹ alkyl-containing groups may be taken together with any nitrogen atom to which they are attached to form a three to forty membered cyclic, heterocyclic or heteroaryl ring;
wherein each R² is independently selected from the group consisting of hydrogen, hydroxyl, halo, —N₃, —CN, —SH, (R¹)₂—N—, (R³)—O—, (R³)—S—, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)hetero-cyclic; wherein each of the aforesaid (C₁-C₆)alkyl, (C₃-C₁₀)cycloalkyl, phenyl, (C₁-C₁₀)heteroaryl and (C₁-C₁₀)heterocyclic substituents for R² may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (Cᵣ C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-n-(C═O), phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)-[((C₁-C₆)alkyl)-N]—, phenyl-(C═O)—NH—, phenyl-(C═O)-[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)-[((C₁-C₆)alkyl)-N]—, [(C₁-C₆)alkyl-]₂N—(C═O)-[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)-[((C₁-C₆)alkyl)-N]—, (C₁-C₆)alkyl-O—(C═O)—NH—, (C₁-C₆)alkyl-O—(C═O)-[((C₁-C₆)alkyl)-N]—, phenyl-O—(C═O)—NH—, phenyl-O—(C═O)-[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein when said R² phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy; and
wherein each R³ is independently selected from the group consisting of hydrogen, (C₃-C₂₀)cycloalkyl, (C₁-C₂₀)alkoxy, (C₁-C₂₀)alkyl, phenyl, (C₁-C₁₀)heteroaryl, (C₁-C₁₀)heterocyclic and (C₃-C₁₀)cycloalkyl; wherein each of the aforesaid (C₁-C₂₀)alkyl, phenyl, (C₁-C₁₀)heteroaryl, (C₁-C₁₀)heterocyclic and (C₃-C₂₀)cycloalkyl substituents for R³ may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂, NO₂, amino, (C₁-C₆)alkyl-amino, [(C₁-C₆)alkyl]₂-amino, (C₁-C₆)alkyl-SO₂(C₁-C₆)alkyl-(C═O)—NH—, (C₁-C₆)alkyl-(C═O)—[((C₁-C₆)alkyl)-N]—, phenyl-(C═O)—NH—, phenyl-(C═O)-[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂—N—(C═O)—, phenyl-NH—(C═O)—, phenyl-[((C₁-C₆)alkyl)-N]-(C═O)—, (C₁-C₁₀)heteroaryl-NH—-(C═O)—, (C₁-C₁₀)heterocyclic-NH—(C═O)—, (C₃-C₁₀)cycloalkyl-NH—(C═O)—, (C₁-C₆)alkyl-(C═O)—O—, and phenyl-(C═O)—O—;
pharmaceutically acceptable salts thereof and pharmaceutically acceptable prodrugs thereof.

14. The compound of claim 1, having the formula: 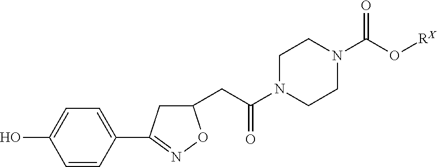
wherein R^{x} is a (C₁-C₆)alkyl, (C₂-C₆)alkenyl, (C₂-C₆)alkynyl, phenyl, (C₁-C₁₀)heteroaryl, (C₁-C₁₀) heterocyclic or (C₃-C₁₀)cycloalkyl group.

15. The compound of claim 1, having the formula: 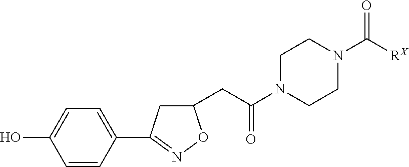
wherein R^{X} is a (C₁-C₆)alkyl, (C₂-C₆)alkenyl, (C₂-C₆)alkynyl, phenyl, (C₁-C₁₀)heteroaryl, (C₁-C₁₀)heterocyclic or (C₃-C₁₀)cycloalkyl group.

16. The compound of claim 1, having the formula: 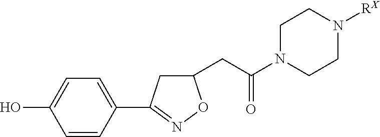
wherein R^{x} is a (C₁-C₆)alkyl, (C₂-C₆)alkenyl, (C₂-C₆)alkynyl, phenyl, (C₁-C₁₀)heteroaryl, (C₁-C₁₀)heterocyclic or (C₃-C₁₀)cycloalkyl group.

17. A method, comprising inhibiting the production of at least one cytokine selected from the group consisting of MIF, IL-1, IL-2, IL-6, IL-8, IFN-γ, TNF, and a combination thereof in a mammalian subject in need thereof by administering an inhibiting-effective amount of the compound of claim 1 to the subject.

18. The method of claim 17, wherein the subject is a human.

19. A method, comprising inhibiting an ERK/MAP pathway in a mammalian subject in need thereof by administering an inhibiting-effective amount of the compound of claim 1 to the subject.

20. The method of claim 19, further comprising treating or preventing at least one ERK/MAP mediated disease selected from the group consisting of psoriatic arthritis, Reiter's syndrome, rheumatoid arthritis, gout, traumatic arthritis, rubella arthritis and acute synovitis, rheumatoid spondylitis, osteoarthritis, gouty arthritis and other arthritic conditions, sepsis, septic shock, endotoxic shock, gram negative sepsis, toxic shock syndrome, Alzheimer's disease, stroke, ischemic and hemorrhagic stroke, neurotrauma/closed head injury, asthma, adult respiratory distress syndrome, chronic obstructive pulmonary disease, cerebral malaria, meningitis, chronic pulmonary inflammatory disease, silicosis, pulmonary sarcostosis, bone resorption disease, osteoporosis, restenosis, cardiac reperfusion injury, brain and renal reperfusion injury, chronic renal failure, thrombosis, glomerularonephritis, diabetes, diabetic retinopathy, macular degeneration, graft vs. host reaction, allograft rejection, inflammatory bowel disease, Crohn's disease, ulcerative colitis, neurodegenerative disease, multiple sclerosis, muscle degeneration, diabetic retinopathy, macular degeneration, tumor growth and metastasis, angiogenic disease, rhinovirus infection, peroral disease, such as gingivitis and periodontitis, eczema, contact dermatitis, psoriasis, sunburn, conjunctivitis, and a combination thereof.

21. A method, comprising inhibiting the production of at least one cytokine selected from the group consisting of MIF, IL-1, IL-2, IL-6, IL-8, IFN-γ, TNF, and a combination thereof in a cell culture by contacting an inhibiting-effective amount of the compound of claim 1 with at least one cell in the cell culture.

22. The method of claim 21, wherein the cell is a human cell.
